(19) United States
(12) Patent Application Publication
(10) Pub. No.: US 2011/0039770 A1
(43) Pub. Date: Feb. 17, 2011

(54) INHIBITORS OF PROTEIN KINASE C ISOFORMS AND USES THEREOF

(75) Inventors: Jenny Phipps, Chelsea (CA); Raphael Terreux, Lyon (FR)

Correspondence Address: DLA PIPER LLP (US), 4365 EXECUTIVE DRIVE, SUITE 1100, SAN DIEGO, CA, 92121-2133, US

(73) Assignee: PHARMAGAP INC., Ottawa, ON (CA)

(21) Appl. No.: 12/063,026

(22) PCT Filed: Aug. 7, 2006
(86) PCT No.: PCT/CA2006/001298

§ 371 Date: Oct. 29, 2010

Related U.S. Application Data
(60) Provisional application No. 60/705,790, filed on Aug. 5, 2005.

(30) Foreign Application Priority Data
Jan. 24, 2006 (CA) 2,532,156
Feb. 22, 2006 (CA) 2,535,622

(51) Int. Cl. A61K 38/16; C07K 7/08; C07K 7/06; C07K 14/00; A61P 35/00; A61P 3/10; A61P 9/00; A61K 38/08; A61K 38/10

(52) U.S. Cl. 514/6.9; 530/327; 530/326; 530/329; 530/324; 530/328; 514/19.3; 514/16.4; 514/19.4; 514/21.7; 514/21.6; 514/21.4; 514/21.3; 514/21.5

(57) ABSTRACT

Inhibitors of mammalian protein kinase C isoforms that comprise an inhibitor moiety, which is capable of inhibiting protein kinase activity, operatively associated with a peptide recognition element (PRE), which has an affinity for one or more PKC isoforms are provided. The targeted inhibitory molecules (TIMs) of the present invention are capable of inhibiting one or more PKC isoforms. The TIMs can be designed to target a specific PKC isoform by selection of a PRE component that is shown to preferentially target that PKC isoform. The TIMs are useful as therapeutic agents in the treatment of PKC-related diseases and disorders, such as cancer, psoriasis, angiogenesis, restenosis, atherosclerosis, cardiovascular disease, hypertension, diabetes, neurological disorders, rheumatoid arthritis, kidney disorders, inflammatory disorders and autoimmune disorders.

### FIELD OF THE INVENTION

The present invention relates to the field of protein kinases and, in particular, to inhibitors of isoforms of protein kinase C.

### BACKGROUND OF THE INVENTION

Protein kinase C enzymes are phospholipid-dependent, cytoplasmic serine/threonine protein kinases that are key players in intracellular signal transduction. As such, PKCs are important mediators of a number of cellular events, including cell growth, differentiation and apoptosis. Due to their involvement in various cellular signalling events, PKCs are of interest to the pharmaceutical and biotech industries as potential drug targets.

There are currently eleven (11) known isoforms of PKC, which have been grouped into three sub-families according to their structure and cofactor regulation. The α, βI, βII and γ isoforms belong to the conventional or classical PKC sub-family; the δ, ε, θ, μ and η isoforms belong to the novel PKC sub-family, and the ζ, and ι/λ isoforms belong to the atypical PKC sub-family. Each isoform is essential, at normal levels, for many cell processes (Dutil, E. M. & Newton, A. C. (2000) J. Biol. Chem., 275 (14), 10697-10701; Newton, A. C. (1995), J. Biol. Chem., 270 (48), 28495-28498).

Although a number of “broad-spectrum” compounds that demonstrate activity towards a range of PKCs have been developed (see Goekjian, P. G. & Jirousek, M. R., ibid.; Goekjian, P. G. & Jirousek, M. R., Expert Opin. Investig. Drugs, 10:2117-2140), identification of “isoform-specific” compounds that demonstrate activity only towards a specific PKC isoform or group of isoforms has proven to be more elusive. Isoforms of PKC are strongly conserved, especially in their catalytic and ATP-binding regions, making selectivity problematic (Xu et al., (2004) J. Biol. Chem., 279 (48), 50401-50409) and the full crystal structure of PKC has yet to be determined.

U.S. Pat. No. 6,165,977 describes isozyme-specific activators/agonists of the PKC-ε isoform. The described activators/agonists are peptides having a sequence corresponding to the region of the PKC-ε protein between amino acids 85 and 92. Peptide inhibitors of PKC-ε have also been described by Johnson (Johnson, J. A., et al., (1996) J. Biol. Chem., 271:24962-24966). These peptides have a sequence that is derived from the V1 region of the PKC-γ protein. U.S. Patent Application No. 2003/0223981 describes peptide inhibitors of the PKC-γ isoform having a sequence derived from the V5 region of the PKC-γ protein, whereas International Patent Application No. PCT/EP93/00816 (WO 93/20101) describes peptide inhibitors that specifically target the PKC zeta isoform.

The α-isoform of PKC (PKC-α) has been implicated in a number of diseases including cancer, cardiovascular disease, diseases of the central nervous system and diabetes (see review by Goekjian, P. G. & Jirousek, M. R., (1999) Curr. Medicinal Chem., 6:877-903; Rosenzweig T, et al. (2002). Diabetes, 51:1921-1930). A role for PKC-α has also been suggested in polycystic kidney disease (Arnould T, et al. (1998) J. Biol. Chem. 13:6013-6018), high blood pressure (Ungvari Z, et al. (2003) Circulation, 108:1253-1258), and multiple sclerosis (Barton A, et al. (2004) Brain, 4:1-6).

Abnormal levels of PKC-α have been noted in a number of human tumours and aberrant over-expression of PKC-α occurs in many types of cancer, including non-small cell lung cancer (Clark et al., (2003) Cancer Research, 63:780-786), ovarian, breast (Lahn et al, (2004) Acta-Haematol. 115:1-8), neuroblastoma, prostate (Powell et al., (1996) Cell Growth and Differentiation; 7:419-428), bladder (Koivunen et al., (2004) Cancer Research 64:5693-5701) and pancreatic cancer (Detjen et al., (2000) J. Cell Sci., 113:3025-3035). In cancer, PKC-α has been implicated in malignant transformation, proliferation, apoptosis, cell migration, cell activation and desensitizing tumour cells to chemotherapeutic agents leading to multi-drug resistance (see review by Hanauske, A-R., et al., (2004), Curr. Pharm. Design, 10:1923-1936; Hofmann, J., (2004) Current Cancer Drug Targets, 4:125-146; Tonetti D, et al. (2003) British J. Cancer, 88:1400-1402).

Compounds known to be capable of targeting the PKC-α isoform include various antibodies, ligands and pseudosubstrates. For example, phorbol esters activate the classical PKC and novel PKC sub-families of PKCs (Brooks G. et al. (1989) Carcinogenesis, 10, 283-288). These esters bind to the same site as the natural activator, diacylglycerol (DAG) (Wright M and McMaster C. (2002) Biol. Res., 35, 223-229). Lipids similar to DAG also bind to this site and exert an activation effect. The protein PICK-1 binds to the PKC-α isoform, but also binds to other proteins (including non-protein kinases). PICK-1 is believed to contribute to PKC intracellular translocation (Wang W-L et al. (2003) J. Biol. Chem. 278, 37705-37712). Another protein, RACK-1 that is present in the plasma membrane binds to activated PKC-α and PKC-β at their C2 domains (Rotenberg S and Sun X-G (1998) J. Biol. Chem., 273, 2390-2395).

A few isoform-selective PKC inhibitors are known that are capable of inhibiting PKC-α activity. For example, UCN-01 (an analogue of staurosporin), GF109203X and Go6976 are selective for classical PKC isoforms (α, βI, βII and γ). Aprinocarsen (also known as LY900003 or Affinitak™), an antisense oligonucleotide, is selective for PKC-α, but targets the mRNA encoding PKC-α rather than the protein itself (see Hanauske, A-R., et al., ibid). UCN-01, bryostatin-1 (a small molecule inhibitor developed by GPC Biotech AG), PKC 412 (a small molecule inhibitor based on staurosporine developed by Novartis and Aprinocarsen have been initiated. However, both bryostatin-1 and UCN-01 were terminated at the Phase II stage, and Aprinocarsen failed a pivotal Phase III trial (no difference in mean survival rate) due to unacceptable half-life kinetics. Phase II trials with PKC 412 are ongoing.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide inhibitors of protein kinase C isoforms and uses thereof. In accordance with one aspect of the present invention, there is provided a targeted protein kinase C inhibitor comprising an inhibitor moiety that is capable of inhibiting the activity of a PKC operatively associated with a peptide of about 5 and about 30 amino acid residues in length, said peptide having a sequence of general formula (I), or the retro form thereof:

X—[(HY—HB)ₙ-linker]ₘ-(HB—HY)₂—HB—(HY)ₘ—Z (I)

wherein:
HY represents a block of 1 to 4 hydrophobic amino acid residues selected from the group of: Ala, Gly, Ile, Leu, Phe and Val;
HB represents a block of 1 to 4 amino acid residues capable of forming hydrogen bonds selected from the group of: Arg, Asn, Asp, Glu, Gln, Lys and Ser;
“linker” represents 1 to 4 Gly residues;
n is 1, 2 or 3;
m is 0 or 1;
X represents the N-terminus of the peptide or a modified version thereof, and
Z represents the C-terminus of the peptide or a modified version thereof.

In accordance with another aspect of the present invention, the inhibitor moiety of the targeted PKC inhibitor is a compound of general formula IX:

(C1)J(M)-N_{y}B_{z}AₓB_{y}N_{y}Bₓ (IX)

wherein:
C1 is NₓB_{y}(A/N)ₓ B_{y}N_{y} and is attached to J by a peptide bond from the N- or C-terminus of C1;
J is 1-4 amino acid residues selected from the group of: Cys, Lys and His;
M is absent or an ATP mimetic moiety optionally linked to an amino acid selected from the group of Ile, Leu, Val or Gly and is attached to J via the side chain or the N-terminus of one of the Lys residues of J or the N-terminus of one of the Cys residues of J;
each N is independently Ala, Ile, Leu, Val or Gly;
each B is independently Arg, Lys or Tyr; and
each A is independently Phe, His or Trp;
each x is independently 0-1;
each y is independently 0-2;
z=0-3, and
the sequence N_{y}B_{z}AₓB_{y}N_{y}Bₓ is 2 or more amino acids in length,
   wherein:
when J comprises one or no Cys residues, the compound of Formula (LX) comprises a single peptide chain and C1 is attached to the N-terminal amino acid of J via a peptide bond from the C-terminus of C1, and
when J comprises two or more Cys residues, at least two of the Cys residues are linked by a disulphide bond and the compound of Formula (IX) thereby comprises a first peptide chain comprising a first of said at least two Cys residues and C1, and a second peptide chain comprising a second of said at least two Cys residues and the sequence —N_{y}B_{z}AₓB_{y}N_{y}Bₓ, and
   wherein if M is absent, the sequence —N_{y}B_{z}AₓB_{y}N_{y}Bₓ contains at least one of Phe or Trp.

In accordance with another aspect of the present invention, there is provided a pharmaceutical composition comprising a targeted protein kinase C inhibitor of the invention and a pharmaceutically acceptable diluent, carrier or excipient.

In accordance with another aspect of the present invention, there is provided a targeted protein kinase C inhibitor of the invention for use in the treatment of a protein kinase C (PKC)-related disease or disorder.

In accordance with another aspect of the present invention, there is provided a use of a targeted protein kinase C inhibitor of the invention for the manufacture of a medicament.

In accordance with another aspect of the present invention, there is provided a method of inhibiting one or more protein kinase C isoforms, said method comprising contacting said one or more PKC isoforms with an effective amount of the targeted PKC inhibitor of the invention.

In accordance with another aspect of the present invention, there is provided a method of treating a mammal having a protein kinase C-related disease or disorder comprising administering to said mammal an effective amount of a targeted PKC inhibitor of the invention.

In accordance with another aspect of the present invention, there is provided a method of treating a mammal having cancer comprising administering to said mammal an effective amount of a targeted PKC inhibitor of the invention.

In accordance with another aspect of the present invention, there is provided a method of increasing the efficacy of a chemotherapeutic agent in a mammal having cancer and undergoing treatment with said chemotherapeutic agent, said method comprising administering to said mammal an effective amount of a targeted PKC inhibitor of the invention.

### BRIEF DESCRIPTION OF THE DRAWINGS

These and other features of the invention will become more apparent in the following detailed description in which reference is made to the appended drawings.

FIG. 1 depicts the subcellular localisation of endogenous PKC-α in (A) untreated human neuroblastoma (IMR-32) cells, (B) IMR-32 cells treated with peptide PRE 4, and (C) IMR-32 cells treated with peptide PRE 3.

FIG. 2 presents the results of a competition binding assay using peptide PRE 1 with various PKC isoforms: (A) PKC-alpha; (B) PKC-beta I, (C) PKC-delta, (D) PKC-iota and (E) PKC-zeta.

FIG. 3 presents the results of a competition binding assay using peptide PRE 4 with various PKC isoforms: (A) PKC-alpha; (B) PKC-beta I, (C) PKC-beta I, (D) PKC-beta II, (E) PKC-delta, (F) PKC-epsilon, (G) PKC-iota and (H) PKC-zeta.

FIG. 4 presents the results of a competition binding assay using peptide PRE 6 with various PKC isoforms: (A) PKC-alpha; (B) PKC-beta I, (C) PKC-delta, (D) PKC-epsilon, (E) PKC-iota and (F) PKC-zeta.

FIG. 5 presents the results of a competition binding assay using peptide PRE 3 with various PKC isoforms: (A) PKC-alpha; (B) PKC-beta I, (C) PKC-beta II, (D) PKC-delta, (E) PKC-epsilon, (F) PKC-epsilon, (G) PKC-iota and (H) PKC-zeta.

FIG. 6 presents the results of a competition binding assay using peptide PRE 7 with various PKC isoforms: (A) PKC-alpha; (B) PKC-beta I, (C) PKC-delta, (D) PKC-epsilon, (E) PKC-iota and (F) PKC-zeta.

FIG. 7 presents the results of a competition binding assay using peptide PRE 8 with various PKC isoforms: (A) PKC-alpha; (B) PKC-beta II, (C) PKC-beta I and (D) PKC-epsilon.

FIG. 8 presents the results of a competition binding assay using peptide PRE 9 with various PKC isoforms: (A) PKC-alpha; (B) PKC-beta I, (C) PKC-beta II, (O) PKC-delta, (E) PKC-epsilon and (F) PKC-zeta.

FIG. 9 presents the results of a competition binding assay using peptide PRE 10 with various PKC isoforms: (A) PKC-alpha; (B) PKC-beta I, (C) PKC-beta II, (D) PKC-delta, (E) PKC-epsilon and (F) PKC-zeta.

FIG. 10 presents the results of a competition binding assay using peptide PRE 11 with various PKC isoforms: (A) PKC-alpha; (B) PKC-beta I, (C) PKC-delta, (D) PKC-epsilon and (E) PKC-zeta.

FIG. 11 presents the results of a competition binding assay using peptide PRE 12 with various PKC isoforms: (A) PKC-alpha; (B) PKC-beta I, (C) PKC-beta II, (D) PKC-delta, (E) PKC-epsilon, (F) PKC-iota and (G) PKC-zeta.

FIG. 12 presents the results of a competition binding assay using peptide PRE 13 with various PKC isoforms: (A) PKC-alpha; (B) PKC-beta I, (C) PKC-delta, (D) PKC-iota, (E) PKC-zeta and (F) PKC-epsilon.

FIG. 13 presents the results of a competition binding assay using peptide PRE 5 with various PKC isoforms: (A) PKC-alpha; (B) PKC-beta I, (C) PKC-beta II, (D) PKC-delta, (E) PKC-epsilon and (F) PKC-zeta.

FIG. 14 presents a schematic diagram of the structure of a protein kinase inhibiting (PM) compound in accordance with one embodiment of the present invention (H atoms omitted).

FIG. 15 depicts the in vitro inhibition of purified PKC-α with PKI compounds 1, 2 and 3.

FIG. 16 depicts the in vitro inhibition of PKC-α sourced from a cell lysate with various doses of (A) PKI compound 1, (B) PM compound 2, and (C) PKI compound 3.

FIG. 17 depicts the effect of compound PKI 3 on apoptosis in MDA-MB-231 breast cancer cells; left hand panels (A, C, E and G) show reverse phase and right hand panels (B, D, F and H) show the nuclei stained with Hoechst reagent.

FIG. 18 depicts the effect of compound PKI 3 on apoptosis in H-661 non-small cell lung cancer cells; left hand panels (A, C, E and G) show reverse phase and right hand panels (B, D, F and H) show the nuclei stained with Hoechst reagent.

FIG. 19 depicts the in vitro inhibition of proliferation of human neuroblastoma cells (IMR-32) with various does of compound TIM 9.

FIG. 20 depicts the morphology of human neuroblastoma cells (IMR-32) treated with compound TIM 9.

FIG. 21 depicts the in vitro inhibition of proliferation of human neuroblastoma cells (IMR-32) with various does of compound TIM 11.

FIG. 22 shows the effect of compound TIM 9 on the phosphorylation of MARCKS peptide by endogenous PKCs in IMR-32 cells after a 30 min incubation.

FIG. 23 shows the effect of compound TIM 9 on the phosphorylation of MARCKS peptide by endogenous PKCs in IMR-32 cells after a 24 hour incubation.

FIG. 24 depicts the in vitro inhibition of proliferation of (A) normal human lung cells (CCD-16Lu), and (B) human NSCLC cells (H661) with various doses of compound TIM 10.

FIG. 25 depicts the in vitro inhibition of proliferation of human neuroblastoma cells (IMR-32) with various doses of compound TIM 10.

FIG. 26 depicts quantitatively the effect of compound TIM 10 on gap junction function in human neuroblastoma cells (IMR-32).

FIG. 27 depicts the effect of compound TIM 10 on the survival of multi-drug resistant human colon cancer cells (LS180).

FIG. 28 depicts the effect of compound TIM 10 on the efflux of fluorescent dyes from multi-drug resistant human colon cancer cells (LS180): (A) calcein AM efflux, and (B) rhodamine 123 efflux.

FIG. 29 depicts quantitatively the effect of compound TIM 10 on calcein AM efflux from multi-drug resistant human colon cancer cells (LS180).

FIG. 30 depicts a comparison of the effects of compound TIM 10 and Verapamil on efflux of rhodamine 123 from multi-drug resistant human colon cancer cells (LS180).

FIG. 31 depicts the levels of connexin 43 (Cx43) and PKC-α proteins in human colon cancer cells (LS180) treated with compound TIM 10 (200× magnification).

FIG. 32 depicts untreated control human NSCLC cells (H661) stained with Hoechst reagent.

FIG. 33 depicts the induction of apoptosis in human NSCLC cells (H661) after internalization of 5 mM compound TIM 10 in Triton X100 at 0.1% in PBS.

FIG. 34 depicts the induction of apoptosis in human NSCLC cells (H661) after internalization of 5 mM compound TIM 10 in PBS.

FIG. 35 depicts the effect of compound TIM 10 on the cell cycle of human NSCLC cells (H661).

FIG. 36 depicts the expression of PKC-α in IMR-32 neuroblastoma cells (A) control cells, (B) control cells treated with 150 ng/ml tetracycline, (C) cells transfected with TIM 17 encoding sequence, and (D) cells transfected with TIM 17 encoding sequence and treated with 150 ng/ml tetracycline.

FIG. 37 depicts the effect of compounds TIM 10, 13, 14 and 15 on proliferation of IMR-32 neuroblastoma cells.

FIG. 38 depicts the effect of addition of TPGS on the toxicity of compounds TIM 10 and 13 in H-69 small cell lung cancer cells.

FIG. 39 depicts a representative (partial ribbon) image of PKCalpha molecule.

FIG. 40 depicts the effect of compound TIM 10 on human LS180 colon cancer cells in mouse xenograft models: (A) shows tumour establishment (M1: tumour size of 2 mm×1-2 min) was delayed approximately 100% (14 days) in mice treated with doxorubicin+compound TIM 10 (5 mg/kg per mouse) versus control cohorts, and (B) shows tumour transition from M1 to M2 (M2: tumour size of 7-8 mm×4-5 mm) was delayed approximately 150% (18 days) in mice treated with doxorubicin+compound TIM 10 (5 mg/kg per mouse) versus control cohorts.

FIG. 41 depicts the effect of compound TIM 15 on tumour establishment in mice subcutaneously injected with MDA-MB-231 breast cancer cells.

FIG. 42 depicts the effect of compound TIM 10 on P-gp and MRP-1 expression in LS180 colon cancer cells; Panel A: control, Panel B: doxorubicin treated cells, and Panel C: cells treated with doxorubicin+compound TIM 10.

FIG. 43 depicts the effect of different doses of compound TIM 10 on human LS180 colon cancer cells in mouse xenograft models, (A) effect on tumour establishment, (B) effect on tumour transition, and (C) effect in tumour progression.

FIG. 44 depicts the effect of compound TIM 10 on protein expression in LS180 colon cancer cells in mouse xenograft models, (A) PKC-α, (B) P-gp, and (C) MRP-1.

FIG. 45 depicts the effect of compound TIM 10 on tumour differentiation as evidenced by CD44 and CD66 expression.

### DETAILED DESCRIPTION OF THE INVENTION

The present invention provides for inhibitors of mammalian protein kinase C (PKC). The targeted inhibitory molecules (TIMs) of the present invention are capable of inhibiting one or more PKC isoforms and comprise an inhibitor moiety, which is capable of inhibiting protein kinase activity, operatively associated with a peptide recognition element (PRE). The PRE has an affinity for one or more PKC isoforms and thus is able to target the inhibitor moiety with which it is associated to these PKC isoform(s).

The type of inhibitor incorporated into the TIM is not critical to the invention provided that the inhibitor moiety is capable of both inhibiting the target PKC and being operatively associated with the PRE. The inhibitor moiety can be a specific PKC inhibitor, a general PKC inhibitor or a broad-spectrum protein kinase inhibitor. In one embodiment of the present invention, the inhibitor moiety is a general PKC inhibitor or a broad-spectrum protein kinase inhibitor. In another embodiment, the inhibitor moiety is a general PKC inhibitor or a broad-spectrum protein kinase inhibitor operatively associated with the PRE via a labile linkage that is cleaved within the cell, thus permitting the released inhibitor molecule to inhibit a number of other protein kinases in the cell, as well as the target PKC.

As indicated above, the PRE incorporated into the TIM of the present invention has an affinity for one or more PKC isoform. Thus the TIMs of the present invention can preferentially or specifically inhibit one PKC isoform, or they can inhibit one or more PKC isoforms. The TIMs of the invention can be designed to target a specific PKC isoform by selection of a PRE component that is shown to preferentially target that PKC isoform. This selectivity can be enhanced, if required, by selecting an inhibitor moiety that shows some specificity towards this isoform. In one embodiment of the present invention, the TIMs inhibit PKC-α and optionally one or more other PKC isoforms. In accordance with this embodiment, the ability of the TIM to inhibit the PKC isoforms other than PKC-α may be equal to or less than the ability to inhibit PKC-α.

In another embodiment, the TIM comprises a PRE that is either specific for PKC-α, or has an affinity for PKC-α and one or more of a sub-group of PKC isoforms consisting of PKC-β (PKC-βI and/or PKC-βII) and PKC-ε. The affinity of the PRE for these other PKC isoforms may be equal to or less than the affinity of the PRE for PKC-α. Accordingly, in one embodiment, the present invention provides for a TIM comprising a PRE that is capable of recognising PKC-α and one or more of PKC-β and PKC-ε. In another embodiment of the present invention, the PRE demonstrates a higher affinity for PKC-α than for other isoforms of PKC. The present invention, therefore, in this embodiment provides for TIMs that specifically target and inhibit PKC-α thereby minimising interaction of the inhibitor moiety with other kinases in the cell.

The TIMs provided by the present invention are capable of inhibiting the activity of one or more PKC isoforms thereby modulating one or more PKC-mediated physiological effects. The TIMs, therefore, are useful as therapeutic agents in the treatment of PKC-related diseases and disorders, such as cancer, psoriasis, angiogenesis, restenosis, atherosclerosis, cardiovascular disease, hypertension, diabetes, neurological disorders, rheumatoid arthritis, kidney disorders, inflammatory disorders and autoimmune disorders. The present invention, therefore, contemplates a method of treating a PKC-mediated disease or disorder in a mammal by administering an effective amount of one or more TIMs. The TIMs can be used alone or in combination with other known therapeutic agents. In one embodiment, the present invention provides for the use of the TIMs in the treatment of cancer. In another embodiment, the present invention provides for the use of the TIMs in combination with one or more conventional chemotherapeutics for the treatment of cancer.

The present invention further contemplates the use of the TIMs as research tools in the development of other PKC inhibitors and to investigate the role of PKCs in various cellular processes and diseases.

The present invention also provides for a method of preparing a PKC inhibitor that specifically targets one isoform of PKC. The method generally comprises the steps of providing a library of candidate isoform-specific PREs, screening the library against one or more PKC isoforms, selecting a PRE having the desired isoform-specificity and conjugating this PRE to a PKC inhibitor.

### DEFINITIONS

Unless defined otherwise, all technical and scientific terms used herein have the same meaning as commonly understood by one of ordinary skill in the art to which this invention belongs.

In the context of the present invention, the term “operatively associated with” means that the inhibitor moiety is connected to the PRE either directly or indirectly via a chemical bond, fusion or an association of sufficient stability to withstand physiological conditions for a sufficient time to allow the TIM to reach its target PKC. A chemical bond can be, for example, one or more of covalent, ionic, disulphide, hydrogen, van der Waals, electrostatic, and the like. When the inhibitor moiety is indirectly connected the PRE, the inhibitor molecule can be connected to a spacer via one of the bonds described above, with the spacer in turn being connected to the PRE via one of the bonds described above, which bond can be the same or different to the bond connecting the inhibitor moiety to the spacer molecule.

The “affinity” of a PRE for a PKC isoform is determined by assaying the ability of the PRE, either alone or incorporated into a TIM of the invention, to interfere with the binding of an antibody specific for the PKC isoform to the target PKC. A PRE that is capable of interfering with the binding of an isoform-specific antibody to its target PKC is defined as having an affinity for that isoform.

The term “interfere with,” as used herein, means to reduce or inhibit.

By “PKC isoform-specific” or “specific for a PKC isoform” as used herein with reference to a PRE or TIM it is meant that the PRE/TIM has a greater affinity for a particular PKC isoform as compared to its affinity for other PKC isoforms when assessed under similar assay conditions, and/or that the PRE/TIM binds to the particular PKC isoform preferentially over other PKC isoforms. Thus, for example, the term “PKC-α specific” or “specific for PKC-α” as used herein with reference to a PRE or TIM of the present invention indicates that the PRE/TIM has a greater affinity for PKC-α than for other PKC isoforms under substantially identical assay conditions, and/or that the PRE/TIM binds to PKC-α preferentially over other PKC isoforms.

The term “naturally-occurring,” as used herein with reference to an object, such as a protein, peptide or amino acid, indicates that the object can be found in nature. For example, a protein, peptide or amino acid that is present in an organism or that can be isolated from a source in nature and which has not been intentionally modified by man in the laboratory is considered to be naturally-occurring.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-p-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. I Pept. Prot. Res. 14:177-185 (1979); Spatola et al., Life Sci. 38:1243-1249 (1986); Hann, J. Chem. Soc. Perkin Trans. 1307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

The term “retro sequence” or “retro peptide,” as used herein, refers to a sequence of amino acids that has been altered with respect to a reference amino acid sequence by a reversal of the direction of the reference amino acid sequence. For example, for a reference sequence “ATPKL,” the retro sequence would be “LKPTA.”

The term “inverso sequence” or “inverso peptide,” as used herein, refers to a sequence of amino acids that has been altered with respect to a reference amino acid sequence in that all L-amino acids of the sequence have been replaced with D-amino acids.

The term “retro-inverso sequence” or “retro-inverso peptide,” as used herein, refers to a sequence of amino acids that has been altered with respect to a reference amino acid sequence in that the amino acid sequence has been reversed and all L-amino acids have been replaced with D-amino acids. Compared to the reference peptide, a retro-inverso peptide has a reversed backbone while retaining substantially the original spatial conformation of the side chains, resulting in an isomer with a topology that closely resembles the reference peptide.

The term “alkyl,” as used herein, refers to a straight chain or branched hydrocarbon of one to ten carbon atoms or a cyclic hydrocarbon group of three to ten carbon atoms. Said alkyl group is optionally substituted with one or more substituents independently selected from the group of: alkyl, alkenyl, alkynyl, aryl, heteroalkyl, aralkyl, hydroxy, alkoxy, aralkyloxy, aryloxy, carboxy, acyl, aroyl, halo, nitro, trihalomethyl, cyano, alkoxycarbonyl, aryloxycarbonyl, aralkoxycarbonyl, acylamino, aroylamino, dialkylamino, carbamoyl, alkylcarbamoyl, dialkylcarbamoyl, alkylthio, aralkylthio, arylthio, alkylene and NZ₁Z₂ where Z₁ and Z₂ are independently hydrogen, alkyl, aryl, and aralkyl. This term is exemplified by such groups as methyl, ethyl, n-propyl, propyl, n-butyl, t-butyl, 1-butyl (or 2-methylpropyl), cyclopropylmethyl, i-amyl, n-amyl, hexyl, cyclopropyl, cyclobutyl, cyclopentyl, and the like.

The term “alkenyl” refers to a straight chain or branched hydrocarbon of two to ten carbon atoms having at least one carbon to carbon double bond. Said alkenyl group can be optionally substituted with one or more substituents as defined above. Exemplary groups include allyl and vinyl.

The term “allynyl” refers to a straight chain or branched hydrocarbon of two to ten carbon atoms having at least one carbon to carbon triple bond. Said alkynyl group can be optionally substituted with one or more substituents as defined above. Exemplary groups include ethynyl and propargyl.

The term “hacroalkyl,” as used herein, refers to an alkyl group of 2 to 10 carbon atoms, wherein at least one carbon is replaced with a hetero atom, such as N, O or S.

The term “aryl” (or “Ar”), as used herein, refers to an aromatic carbocyclic group containing about 6 to about 10 carbon atoms or multiple condensed rings in which at least one ring is aromatic carbocyclic group containing 6 to about 10 carbon atoms. Said aryl or Ar group can be optionally substituted with one or more substituents as defined above. Exemplary aryl groups include phenyl, tolyl, xylyl, biphenyl, naphthyl, 1,2,3,4-tetrahydronaphthyl, anthryl, phenanthryl, 9-fluorenyl, and the like.

The term “aralkyl,” as used herein, refers to a straight or branched chain alkyl, alkenyl or alkynyl group, wherein at least one of the hydrogen atoms is replaced with an aryl group, wherein the aryl group can be optionally substituted with one or more substituents as defined above. Exemplary aralkyl group include benzyl, 4-phenylbutyl, 3,3-diphenylpropyl and the like.

The term “alkoxy,” as used herein, refers to RO—, wherein R is alkyl, alkenyl or alkynyl in which the alkyl, alkenyl and alkynyl groups are as previously described. Exemplary alkoxy groups include methoxy, ethoxy, n-propoxy, I-propoxy, n-butoxy, and heptoxy.

The term “aryloxy” as used herein, refers to an “aryl-O—” group in which the aryl group is as previously described. Exemplary aryloxy groups include phenoxy and naphthoxy.

The term “alkylthio,” as used herein, refers to RS—, wherein R is alkyl, alkenyl or alkynyl in which the alkyl, alkenyl and alkynyl groups are as previously described. Exemplary alkylthio groups include methylthio, ethylthio, 1-propylthio and hepthylthio.

The term “arylthio,” as used herein, refers to an “aryl-S—” group in which the aryl group is as previously described. Exemplary arylthio groups include phenylthio and naphthylthio.

The term “aralkyloxy,” as used herein, refers to an “aralkyl-O—” group in which the aralkyl group is as previously described. Exemplary aralkyloxy groups include benzyloxy.

The term “aralkylthio,” as used herein, refers to an “aralkyl-S—” group in which the aralkyl group is as previously described. Exemplary aralkylthio groups include benzylthio.

The term “dialkylamino,” as used herein, refers to an —NZ₁Z₂ group wherein Z₁ and Z₂ are independently selected from alkyl, alkenyl or alkynyl, wherein alkyl, alkenyl and alkynyl are as previously described. Exemplary dialkylamino groups include ethylmethylamino, dimethylamino and diethylamino.

The term “alkoxycarbonyl,” as used herein, refers to R—O—CO—, wherein R is alkyl, alkenyl or alkynyl, wherein alkyl, alkenyl and alkynyl are as previously described. Exemplary alkoxycarbonyl groups include methoxy-carbonyl and ethoxy-carbonyl.

The term “aryloxycarbonyl,” as used herein, refers to an “aryl-O—CO—”, wherein aryl is as defined previously. Exemplary aryloxycarbonyl groups include phenoxy-carbonyl and naphtoxy-carbonyl.

The term “aralkoxycarbonyl,” as used herein, refers to an “aralkyl-O—CO—,” wherein aralkyl is as defined previously. Exemplary aralkoxycarbonyl groups include benzyloxycarbonyl.

The term “acyl” as used herein, refers to RC(O)—, wherein R is alkyl, alkenyl, alkynyl, heteroalkyl, a heterocyclic ring, or a heteroaromatic ring, wherein alkyl, alkenyl, alkynyl, heteroalkyl, heterocyclic, and heteroaromatic are as defined previously.

The term “aroyl” as used herein, refers to an ArC(O)— group, wherein Ar is as defined previously.

The term “carboxy” as used herein, refers to ROC(O)—, wherein R is H, alkyl, alkenyl or alkynyl, and wherein alkyl, alkenyl or alkynyl are as defined previously.

The term “carbamoyl,” as used herein, refers to a H₂N—CO— group.

The term “alkylcarbamoyl,” as used herein, refers to an “Z₁Z₂N—CO—” group wherein one of the Z₁ and Z₂ is hydrogen and the other of Z₁ and Z₂ is independently selected from alkyl, alkenyl or alkynyl and wherein alkyl, alkenyl and alkynyl are as defined previously.

The term “dialkylcarbamoyl,” as used herein, refers to a “Z₁Z₂N—CO—” group wherein Z₁ and Z₂ are independently selected from alkyl, alkenyl or alkynyl and wherein alkyl, alkenyl and alkynyl are as defined previously.

The term “acylamino”, as used herein, refers to an “acyl-NH—” group, wherein acyl is as defined previously.

The term “halo” as used herein, refers to fluoro, chloro, bromo or iodo. In one embodiment, “halo” refers to fluoro, chloro or bromo.

Other chemistry terms herein are used according to conventional usage in the art, as exemplified by The McGraw-Hill Dictionary of Chemical Terms (ed. Parker, S., 1985), McGraw-Hill, San Francisco).

The term “reactive functionality,” as used herein, refers to a chemical group present on a first molecule that is capable of bonding to, or can be modified and/or activated to be capable of bonding to, a second molecule.

The terms “therapy” and “treatment,” as used interchangeably herein, refer to an intervention performed with the intention of improving a subject's status. The improvement can be subjective or objective and is related to ameliorating the symptoms associated with, preventing the development of, or altering the pathology of a disease or disorder being treated. Thus, the terms therapy and treatment are used in the broadest sense, and include the prevention (prophylaxis), moderation, reduction, and curing of a disease or disorder at various stages. Preventing deterioration of a subject's status is also encompassed by the term. Subjects in need of therapy/treatment thus include those already having the disease or disorder as well as those prone to, or at risk of developing, the disease or disorder and those in whom the disease or disorder is to be prevented.

The term “ameliorate” includes the arrest, prevention, decrease, or improvement in one or more the symptoms, signs, and features of the disease or disorder being treated, either temporarily or in the long-term.

The term “subject” or “patient” as used herein refers to a mammal in need of treatment.

Administration of the TIMs of the invention “in combination with” one or more further therapeutic agents, is intended to include simultaneous (concurrent) administration and consecutive administration. Consecutive administration is intended to encompass administration of the therapeutic agent(s) and the TIM(s) of the invention to the subject in various orders and via various routes.

As used herein, the term “about” refers to a +/−10% variation from the nominal value. It is to be understood that such a variation is always included in any given value provided herein, whether or not it is specifically referred to.

Naturally-occurring amino acids are identified throughout by the conventional three-letter or one-letter abbreviations indicated below, which are as generally accepted in the peptide art and are recommended by the IUPAC-IUB commission in biochemical nomenclature:

TABLE 1
Amino acid codes

| | 3-letter | 1-letter |
|---|---|---|
| Name | code | code |
| Alanine | Ala | A |
| Arginine | Arg | R |
| Asparagine | Asn | N |
| Aspartic | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The peptide sequences set out herein are written according to the generally accepted convention whereby the N-terminal amino acid is on the left and the C-terminal amino acid is on the right. By convention also, L-amino acids are represented by upper case letters and D-amino acids by lower case letters.

### Targeted Inhibitory Molecules

As indicated above, the targeted inhibitory molecules (TIMs) of the present invention comprise an inhibitor moiety, which is capable of inhibiting protein kinase activity, operatively associated with a peptide recognition element (PRE), which has an affinity for one or more PKC isoform and thus is able to target the inhibitor moiety with which it is associated to the target PKC isoform(s).

The operative association between the inhibitor molecule and the PRE can be a strong association such that the two entities do not readily dissociate under physiological conditions, or it can be a weak (or labile) association that allows the two entities to dissociate rapidly under physiological conditions.

The TIMs of the present invention thus comprise an inhibitor moiety and a PRE, and optionally a spacer. As described in more detail below, the PRE is a peptide of defined structure and the inhibitor moiety can be one of a number of protein kinase inhibitors known in the art, which may be peptidic or non-peptidic. Similarly, when present, the spacer can be a peptidic spacer or a non-peptidic spacer. Accordingly, in one embodiment of the present invention the TIM is entirely peptidic. In another embodiment of the present invention, the TIM is a mixture of peptidic and non-peptidic components.

The TIM can comprise one or more additional components conjugated to either the inhibitor molecule or the PRE. Additional components can also be conjugated to the spacer, when present. Such additional components can act to stabilise the TIM, provide additional targeting, provide a detectable label, facilitate preparation, isolation and/or purification of the TIM, promote or facilitate cellular uptake, increase the physiological half-life of the TIM, and the like. Various compounds known in the art can be conjugated to the TIM for the purposes specified above.

The present invention further contemplates that the TIMs can be targeted to a specific PKC isoform, or to a group of isoforms, through the selection of the appropriate PRE. Specificity can be refined, if desired, by selection of an inhibitor moiety that demonstrates specificity for the isoform or isoforms of interest.

### 1. Peptide Recognition Elements

The peptide recognition elements (PREs) included in the TIMs of the present invention are peptides between about 5 and about 30 amino acid residues in length and have a sequence represented by general formula (I), or the retro form thereof (general formula (I-R)):

X—[(HY—HB)ₙ-linker]ₘ—(HB—HY)₂—HB—(HY)ₘ—Z (I)

X—(HY)ₘ—HB—(HY—HB)₂-[linker-(HB—HY)ₙ]ₘ—Z (I-R)

wherein:
HY represents a block of 1 to 4 hydrophobic amino acid residues selected from the group of: Ala, Gly, Ile, Leu, Phe and Val;
HB represents a block of 1 to 4 amino acid residues capable of forming hydrogen bonds selected from the group of: Arg, Asn, Asp, Glu, Gln, Lys and Ser;
“linker” represents 1 to 4 Gly residues;
n is 1, 2 or 3;
m is 0 or 1;
X represents the N-terminus of the peptide or a modified version thereof, and
Z represents the C-terminus of the peptide or a modified version thereof.

In one embodiment, the PREs included in the TIMs the present invention have a sequence represented by general formula (II), or the retro form thereof (general formula (II-R)):

X—[(HY—HB1)ₙ-linker]ₘ-(HB—HY)₂—HB2-(HY)ₘ—Z (II)

X—(IIY)ₘ—HB2-(HY—HB)₂-[linker-(HB1-HY)ₙ]ₘ—Z (II-R)

wherein:
HY, HB, “linker,” n, m, X and Z are as defined above for formula (I), and
HB1 and HB2 represent sub-blocks of a HB block, wherein HB1 consists of 1 to 3 amino acid residues selected from the group specified above for HB and HB2 consists of 1 or 2 amino acid residues selected from the group specified above for HB.

In another embodiment of the present invention, the “linker” in formula (II) or (II-R) represents 1 to 3 Gly residues. In a further embodiment, the “linker” in formula (II) or (II-R) represents 1 or 2 Gly residues.

In another embodiment, the PREs included in the TIMs of the present invention have a sequence represented by general formula (III), or the retro form thereof (general formula (III-R)):

X—(HB—HY)₂—HB2-(HY)ₘ—Z (III)

X—(HY)ₘ—HB2-(HY—HB)₂—Z (III-R)

wherein:
HY, HB, HB2, m, X and Z are as defined above for formula (II).

In another embodiment, the PREs have a sequence represented by general formula (IV), or the retro form thereof (general formula (IV-R)):

X—(HB—HY)₂—HB2-Z (IV)

X—HB2-(HY—HB)₂—Z (IV-R)

wherein:
HY, HB, HB2, X and Z are as defined above for formula (III).

In another embodiment, the PREs included in the TIMs of the present invention have a sequence represented by general formula (V), or the retro form thereof (general formula (V-R)):

X—(HB—HY)₂—HB2-HY—Z (V)

X—HY—HB2-(HY—HB)₂—Z (V-R)

wherein:
HY, HB, HB2, X and Z are as defined above for formula (III).

In another embodiment of the present invention, in formula (V) or (V-R), HB consists of 1 or 2 amino acid residues selected from the group specified above for HB. In a further embodiment, in formula (V) or (V-R), HB2 consists of 1 amino acid residue selected from the group specified above for HB.

In an alternative embodiment of the present invention, the PREs have a sequence represented by general formula (VI), or the retro form thereof (general formula (VI-R)):

X—(HY—HB1)ₙ-linker-(HB—HY)₂—HB2-(HY)ₘ—Z (VI)

X—(HY)ₘ—HB2-(HY—HB)₂-linker-(HB1-HY)ₙ—Z (VI-R)

wherein:
HY, MB, HB1, HB2, “linker,” n, m, X and Z are as defined above for formula (II).

In another embodiment, the PREs have a sequence represented by general formula (VII), or the retro form thereof (general formula (VII-R)):

X—(HY—HB1)₃-linker-(HB—HY)₂—HB2-HY—Z (VII)

X—HY—HB2-(HY—HB)₂-linker-(HB1-HY)₃—Z (VII-R)

wherein:
HY, HB, HB1, HB2, “linker,” X and Z are as defined above for formula (VI).

In another embodiment of the present invention, in formula (VII) or (VII-R), HB and HB1 consist of 1 or 2 amino acid residues selected from the group specified above for HB. In a further embodiment, in formula (VII) or (VII-R), HB2 consists of 1 amino acid residue selected from the group specified above for FIB.

In another embodiment of the present invention, the PREs have a sequence represented by general formula (VIII), or the retro form thereof (general formula (VIII-R)):

X—HY—HB1-linker-(HB—HY)₂—HB2-HY—Z (VIII)

X—HY—HB2-(HY—HB)₂-linker-HB1-HY—Z (VIII-R)

wherein:
HY, HB, HB1, HB2, “linker,” X and Z are as defined above for formula (VI).

In another embodiment of the present invention, in formula (VIII) or (VIII-R), HB consists of 1 or 2 amino acid residues selected from the group specified above for HB. In a further embodiment, in formula (VIII) or (VIII-R), HB2 consists of 1 amino acid residue selected from the group specified above for HB.

In another embodiment of the present invention, in formula (VI), (VI-R), (VII), (VII-R), (VIII) or (VIII-R), “linker” represents 1 to 3 Gly residues. In a further embodiment, in formula (VI), (VI-R), (VII), (VII-R), (VIII) or (VIII-R), “linker” represents 1 or 2 Gly residues.

In a further embodiment of the present invention, the PREs are less than about 25 amino acids residues in length. In another embodiment, the PREs are between about 5 and about 25 amino acid residues in length. In a further embodiment, the PREs are between about 6 and about 25 amino acid residues in length. In another embodiment, the PREs are between about 7 and about 25 amino acid residues in length. In another embodiment, the PREs are less than about 22 amino acids in length. In other embodiments, the PREs are between about 5 and about 22 amino acid residues in length; between about 6 and about 22 amino acid residues in length; between about 7 and about 22 amino acid residues in length; between about 7 and about 20 amino acid residues in length; between about 8 and about 20 amino acid residues in length and between about 10 and about 20 amino acid residues in length.

The present invention also contemplates PREs that are retro, inverso, or retro-inverso forms of any one of formulae (I), (II), (III), (IV), (V), (VI), (VII) or (VIII). In one embodiment of the present invention, the PRE has a sequence that is the retro form of general formula (I). In another embodiment, the PRE has a sequence that is the inverso form of general formula (I). In a further embodiment, the PRE has a sequence that is the retro-inverso form of general formula (I). In another embodiment, the PRE has a sequence that is the retro, inverso or retro-inverso form of general formula (III).

X and Z in formulae (I), (I-R), (II), (II-R), (III), (III-R), (IV), (IV-R), (V), (V-R), (VI), (VI-R), (VII), (VII-R), (VIII) and (VIII-R) above can represent a free amino (N)-terminus and a free carboxy (C)-terminus, respectively, or a modified N-terminus and C-terminus. The PREs can thus have a modified N-terminus, a modified C-terminus, or both a modified N-terminus and a modified C-terminus. Examples of chemical substituent groups suitable for modifying the N-terminus and/or C-terminus of peptides are known in the art and include, but are not limited to, alkyl, alkenyl, alkynyl, amino, aryl, aralkyl, heteroalkyl, hydroxy, alkoxy, aralkyloxy, aryloxy, carboxy, acyl, amyl, halo, nitro, alkoxycarbonyl, aryloxycarbonyl, aralkoxycarbonyl, acylamino, aroylamino, dialkylamino, carbamoyl, alkylcarbamoyl, dialkylcarbamoyl, alkylthio, aralkylthio, arylthio, alkylene, and NZ₁Z₂ where Z₁ and Z₂ are independently hydrogen, alkyl, aryl, or aralkyl, and the like. Blocking groups such as Fmoc (fluorenylmethyl-O—CO—), carbobenzoxy (benzyl-O—CO—), monomethoxysuccinyl, naphthyl-NH—CO—, acetylamino-caproyl and adamantyl-NH—CO—, can also be used. Other modifications contemplated by the present invention include C-terminal amidation, esterification, hydroxymethyl modification and O-modification (for example, C-terminal hydroxymethyl benzyl ether), as well as N-terminal modifications such as substituted amides, for example alkylamides and hydrazides.

In one embodiment of the present invention, X represents a N-terminus modified with an acyl group. Non-limiting examples of suitable acyl groups are benzoyl, acetyl, t-butylacetyl, p-phenylbenzoyl, trifluoroacetyl, cyclohexylcarbonyl, phenylacetyl, 4-phenylbutanoyl, 3,3-diphenylpropanoyl, 4-biphenylacetyl, diphenylacetyl, 2-naphthylacetyl, 3-phenylbutanoyl, α-phenyl-ortho-toluoyl, indole-3-acetyl, 3-indolepropanoyl, 3-indolebutanoyl, 4-(4-methoxyphenyl)butanoyl, and the like. In another embodiment, X represents a N-terminus modified with an acetyl group. In another embodiment, Z represents a C-terminus modified with an amino group.

The PRE can comprise one or more non-naturally occurring amino acids. Suitable non-naturally occurring amino acids are known in the art and include those listed above. One skilled in the art could readily select appropriate non-naturally occurring amino acids for inclusion in the PRE based on consideration of the characteristics of the natural amino acid to be replaced, such as charge, size, polarity, hydrophobicity, and the like. When the PRE comprises more than one non-naturally occurring amino acid, the non-naturally occurring amino acids can be the same or different. In one embodiment, the PRE comprises one or more D-amino acid. In another embodiment, the PRE is an inverso sequence, i.e. contains all D-amino acids.

The amino acid residues included in the PREs of the invention are linked together by peptide bonds. The peptide bonds can be naturally-occurring or non-naturally occurring (modified) peptide bonds. Examples of suitable modified peptide bonds are known in the art and include those listed above. The PRE can comprise one or more modified peptide bonds. When the PRE comprises more than one modified peptide bond, the modified peptide bonds can be the same or different.

Representative, non-limiting examples of PREs suitable for inclusion in the TIMs of the present invention are provided in Table 2. In a specific embodiment, the PRE is less than about 30 amino acid residues in length that comprises an amino acid sequence selected from the group of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 and SEQ ID NO:35, or the retro, inverso, or retro-inverso form thereof, wherein each of the N-terminus and C-terminus of the PRE are independently either free or modified. In another embodiment, the PRE is less than about 30 amino acids in length that comprises an amino acid sequence selected from the group of: PRE 1, PRE 2, PRE 3, PRE 4, PRE 5, PRE 6, PRE 7, PRE 8, PRE 9, PRE 10, PRE 11, PRE 12, PRE 13, PRE 14, PRE 15, PRE 16, PRE 17, PRE 18, PRE 19, PRE 20, PRE 21, PRE 22, PRE 23, PRE 24, PRE 25, PRE 26, PRE 27, PRE 28, PRE 29, PRE 30, PRE 31, PRE 32, PRE 33, PRE 34 and PRE 35 (as shown in Table 2). In a further embodiment, the PRE is less than about 30 amino acids in length that comprises an amino acid sequence selected from the group of PRE 1, PRE 2, PRE 3, PRE 4, PRE 5, PRE 6, PRE 7, PRE 8, PRE 9, PRE 10, PRE 11, PRE 12, PRE 13, PRE 14, PRE 15, PRE 16, PRE 17, PRE 18, PRE 19, PRE 20, PRE 21, PRE 22, PRE 23, PRE 24 and PRE 25 (as shown in Table 2).

TABLE 2
Exemplary PRE Sequences

| PRE # | Sequence | SEQ ID NO |
|---|---|---|
| 1 | RRKKGGKDFVVKR | 1 |
| 14 | KDAQNLIGISI | 2 |
| 15 | KDANQLIGISI | 3 |
| 16 | AKGIQEVKGGDAQNLIGISI | 4 |
| 8 | ILEDKGGDAQNLIGISI | 5 |
| 17 | RDAQNLIGISI | 6 |
| 18 | AKGIQEVKGGKDAQNLIGISI | 7 |
| 19 | KDAQNLIGISL | 8 |
| 20 | KDAQNLI | 9 |
| 21 | RDAQNLI | 10 |
| 2 | KDAQNLIGISL-NH₂ | 11 |
| 3 | Ac-AKGIQEVKGGDAQNLIGISI-NH₂ | 12 |
| 4 | Ac-KDAQNLIGISI-NH₂ | 13 |
| 5 | Ac-AKGIQEVKGGKDAQNLIGISI-NH₂ | 14 |
| 22 | Dansylglycine-KDAQNLIGISI-NH₂ | 15 |
| 6 | Ac-KDANQLIGISI-NH₂ | 16 |
| 7 | Ac-ISIGILQNADK-NH₂ | 17 |
| 9 | Ac-isigilqnadk-NH₂ | 18 |
| 10 | Ac-ISIGILNQADK-NH₂ | 19 |
| 11 | Ac-RDAQNLIGISI-NH₂ | 20 |
| 12 | Ac-KDAQNLI-NH₂ | 21 |
| 13 | Ac-RDAQNLI-NH₂ | 22 |
| 23 | ISIGILQNADK | 23 |
| 24 | isigilqnadk | 24 |
| 25 | ISIGILNQADK | 25 |
| 26 | RRRRGQQNNLS | 26 |
| 27 | KKKKGGNLVKRIL | 27 |
| 28 | ARIQQEILKKRGGGKDAQNLIGISL | 28 |
| 29 | ARGIQEFRGGKEAQNLVISIL | 29 |
| 30 | REAQNLIGISI | 30 |
| 31 | EAQNLIGISI | 31 |
| 32 | EAQNVIVISIL | 32 |
| 33 | EAQVSI | 33 |
| 34 | KAQNISI | 34 |
| 35 | RDAQVVRIV | 35 |

The present invention also contemplates PREs having a sequence that is a chimeric form of general formula (I), i.e. comprises two or more sequences of general formula (I) joined together. In one embodiment, therefore, the present invention provides for a PRE of less than about 30 amino acid residues in length that comprises one or more of the amino acid sequences: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 and SEQ ID NO:35, or the retro, inverso, or retro-inverso form thereof, wherein each of the N-terminus and C-terminus of the PRE are independently either free or modified.

### II. Inhibitor Moiety

Various known protein kinase inhibitors can be included in the TIM of the present invention as the inhibitor moiety. In general, a protein kinase inhibitor is selected that is capable of inhibiting one or more PKC isoforms. In this regard, the inhibitor moiety can be a broad-spectrum protein kinase inhibitor that is capable of inhibiting PKC-α and other protein kinases, a PKC-specific inhibitor that is capable of inhibiting one or more PKC isoforms, or a PKC-isoform specific inhibitor that is capable of inhibiting a specified PKC isoform.

A wide range of protein kinase inhibitors are known in the art and many are commercially available (for example from Biaffin GmbH & Co KG, Kassel, Germany; EMD Biosciences, San Diego, Calif., and Sigma-Aldrich, St. Louis, Mo.). Examples of suitable protein kinase inhibitors for include, but are not limited to, Apigenin; Bisindolylmaleimide I, II, III, IV and V; Calphostin C; Cardiotoxin (Naja nigricollis); Chelerythrine; Choline hexadecyl phosphate; Dequalinium chloride; Edelfosine (also known as edelfosina or ET18OCH3); Ellagic acid; Genistein; Gö 6976; H-7 (1-(5-isoquinolinesulfonyl)-2-methylpiperazine); H8 (N-[2-(methylamino)ethyl]-5-isoquinolinesulfonamide); H-9 (N-(2-aminoethyl)-5-isoquinolinesulfonamide); H-89 (N-[2-(p-bromocinnamylamino)ethyl]-5-isoquinolinesulfonamide); HA-100 (1-(5-isoquinolinesulfonyl)piperazine); HA-1004 (N-(2-guanidinoethyl)-5-isoquinolinesulfonamide); HBDDE (2,2′,3,3′,4,4′-hexahydroxy-1,1′-biphenyl-6,6′-dimethanol dimethyl ether); Hispidin; Hypericin; K-252a; Melittin; ML-7 (1-(5-iodonaphthalene-1-sulfonyl)-1H-hexahydro-1,4-diazepine); Myricetin; NGIC-I (non-glyosidic indolocarbazole I); Palmitoyl-DL-carnitine; Phloretin; Piceatannol; Polymyxin B sulphate; Protein kinase C fragment 19-36; Pseudo hypericin; Quercetin; Rottlerin; Sangivamycin; Staurosporin; Tamoxifen and TER14687.

In one embodiment of the present invention, the inhibitory moiety is a protein kinase inhibiting (PKI) compound that comprises between about 5 and about 20 amino acids and have the general Formula (LX):

(C1)J(M)-N_{y}B_{z}AₓB_{y}N_{y}Bₓ (IX)

wherein:
C1 is NₓB_{y}(A/N)ₓ B_{y}N_{y} and is attached to J by a peptide bond from the N- or C-terminus of C1;
J is 1-4 amino acid residues selected from the group of Cys, Lys and His;
M is absent or an ATP mimetic moiety optionally linked to an amino acid selected from the group of Ile, Leu, Val or Gly and is attached to J via the side chain or the N-terminus of one of the Lys residues of J or the N-terminus of one of the Cys residues of J;
each N is independently Ala, Ile, Leu, Val or Gly;
each B is independently Arg, Lys or Tyr; and
each A is independently Phe, His or Trp;
each x is independently 0-1;
each y is independently 0-2;
z=0-3, and
the sequence N_{y}B_{z}AₓB_{y}N_{y}Bₓ is 2 or more amino acids in length,
   wherein:
when J comprises one or no Cys residues, the compound of Formula (IX) comprises a single peptide chain and C1 is attached to the N-terminal amino acid of J via a peptide bond from the C-terminus of C1, and
when J comprises two or more Cys residues, at least two of the Cys residues are linked by a disulphide bond and the compound of Formula (IX) thereby comprises a first peptide chain comprising a first of said at least two Cys residues and C1, and a second peptide chain comprising a second of said at least two Cys residues and the sequence —N_{y}B_{z}AₓB_{y}N_{y}Bₓ, and
   wherein if M is absent, the sequence —N_{y}B_{z}AₓB_{y}N_{y}Bₓ contains at least one of Phe or Trp.

One skilled in the art will appreciate that when M comprises an Ile, Leu, Val or Gly residue, the ATP moiety can be directly linked to J, or it can be attached to J via the Ile, Leu, Val or Gly residue. Similarly, when J comprises two Cys residues linked by a disulphide bond and one or two other amino acids selected from Cys, His or Lys, C1 and the sequence —N_{y}B_{z}AₓB_{y}N_{y}Bₓ can be attached directly to the respective Cys residues making up the disulphide bond, or via one or more intervening Cys, His or Lys residues.

In one embodiment of the present invention, the PKI compounds of Formula (IX) comprise a modified N-terminus and/or C-terminus.

In another embodiment of the present invention, the PKI compound of Formula (IX) is modified at a C-terminus to include a “tag” of between 1 to 4 amino acids in length that comprises one or more acidic amino acid residues. Other non-acidic amino acid residues included in the tag are selected from the group of: Gly, Val, Ile, Leu and Lys. Examples of suitable tags that can be added at the C-terminus of the PKI compounds include, but are not limited to, Glu-Val-Glu; Asp-Asp, Glu-Gly-Glu; Glu-Ile-Glu; Glu-Val-Glu-Lys and Glu-Val-Asp.

In another embodiment, J comprises two Cys residues linked by a disulphide bond and the compound of Formula (IX) thereby comprises a first peptide chain comprising C1 attached to the N- or C-terminus of the first of said two Cys residues, and a second peptide chain comprising the sequence —N_{y}B_{z}AₓB_{y}N_{y}Bₓ attached to the C-terminus the second of said two Cys residues.

In another embodiment, J comprises two Cys residues linked by a disulphide bond and the compound of Formula (IX) thereby comprises a first peptide chain comprising C1 attached to the C-terminus of the first of said two Cys residues, and a second peptide chain comprising the sequence —N_{y}B_{z}AₓB_{y}N_{y}Bₓ attached to the C-terminus the second of said two Cys residues.

In another embodiment of the present invention, in the PKI compounds of Formula (IX), each of C1 and —N_{y}B_{z}AₓB_{y}N_{y}Bₓ are two or more amino acid residues in length. In another embodiment of the present invention, in the PKI compounds of Formula (I), —N_{y}B_{z}AₓB_{y}N_{y}Bₓ is 3 or more amino acid residues in length. In a further embodiment, at least one of C1 and —N_{y}B_{z}AₓB_{y}N_{y}Bₓ is 3 or more amino acid residues in length. In another embodiment, both C1 and —N_{y}B_{z}AₓB_{y}N_{y}Bₓ are 3 or more amino acid residues in length. In another embodiment, each of C1 and N_{y}B_{z}AₓB_{y}N_{y} are 4 or more amino acid residues in length.

In another embodiment of the present invention, the PKI compounds of Formula (IX) have the general Formula (X):

(C1)J(M)-N_{y}B_{z}AₓB_{y}N_{y} (X)

wherein:
C1 is NₓB_{y}(A/N)ₓ B_{y}N_{y} and is attached to J by a peptide bond from the N- or C-terminus of C1;
J is 1-4 amino acid residues selected from the group of: Cys, Lys and His;
M is absent or an ATP mimetic moiety optionally linked to an amino acid selected from the group of Ile, Leu, Val or Gly and is attached to J via the side chain or the N-terminus of one of the Lys residues of J or the N-terminus of one of the Cys residues of J;
each N is independently Ala, Ile, Leu, Val or Gly;
each B is independently Arg, Lys or Tyr; and
each A is independently Phe, His or Trp;
each x is independently 0-1;
each y is independently 0-2;
z=0-3, and
the sequence N_{y}B_{z}AₓB_{y}N_{y} is 2 or more amino acids in length, and
   wherein:
when J comprises one or no Cys residues, the compound of Formula (X) comprises a single peptide chain and C1 is attached to the N-terminal amino acid of J via a peptide bond from the C-terminus of C1, and
when J comprises two or more Cys residues, at least two of the Cys residues are linked by a disulphide bond and the compound of Formula (X) thereby comprises a first peptide chain comprising a first of said at least two Cys residues and C1, and a second peptide chain comprising a second of said at least two Cys residues and the sequence —N_{y}B_{z}AₓB_{y}N_{y}B_{z}AₓB_{y}N_{y}Bₓ.

In another embodiment of the present invention, the PKI compounds of Formula (IX) have the general Formula (XI):

(C2)J(M)-N_{y}B_{z}AₓB_{y}N_{y} (XI)

wherein:
C2 is B_{y}(A/N)ₓ, B_{y}N_{y} and is attached to J by a peptide bond from the N- or C-terminus of C2;
J comprises two Cys residues and optionally 1-2 residues selected from His and Lys, the Cys residues are linked by a disulphide bond and the compound of Formula (I) thereby comprises a first peptide chain comprising a first of said two Cys residues and C2, and a second peptide chain comprising a second of said two Cys residues and the sequence —N_{y}B_{z}AₓB_{y}N_{y}Bₓ,
M is an ATP mimetic moiety optionally linked to an amino acid selected from the group of Ile, Leu, Val or Gly and is attached to J via the N-terminus of one of the Cys residues of J; and
N, B, A, x, y and z are as defined for Formula (IX) above.
   In a further embodiment, the PKI compounds have the general Formula (XI) wherein:
J comprises two Cys residues and optionally 1-2 residues selected from His and Lys, the Cys residues are linked by a disulphide bond and the compound of Formula (XI) thereby comprises a first peptide chain comprising C2 attached to the C-terminus of a first of said two Cys residues, and a second peptide chain comprising the sequence —N_{y}B_{z}AₓB_{y}N_{y}Bₓ attached to the C-terminus of a second of said two Cys residues.

In another embodiment of the present invention, the PKI compounds of Formula (IX) have the general Formula (XII):

NₓB_{y}(A/N)ₓB_{y}N_{y}-J(M)-N_{y}B_{z}AₓB_{y}N_{y}Bₓ (XII)

wherein:
J is 1-2 Lys residues or a Cys residue;
M is absent or is an ATP mimetic moiety attached to J via the side chain of one of the Lys residues of J or the N-terminus of the cysteine residue of J; and
N, B, A, x, y and z are as defined for Formula (IX) above.

In another embodiment of the present invention, the PKI compounds of Formula (IX) have the general Formula (XIII):

NₓB_{y}(A/N)ₓB_{y}N_{y}-J(M)-N_{y}B_{z}AₓB_{y}N_{y} (XIII)

wherein:
J is 1-2 Lys residues;
M is an ATP mimetic moiety attached to J via the side chain of one of the Lys residues; and
N, B, A, x, y and z are as defined for Formula (IX) above.

In another embodiment of the present invention, the PKI compounds of Formula (IX) have the general Formula (XIV):

NₓB_{y}(A/N)ₓB_{y}N_{y}-J-N_{y}B_{z}AB_{y}N_{y} (XIV)

wherein:
J comprises a Cys residue and optionally 1-2 residues selected from His and Lys; and
N, B, A, x, y and z are as defined for Formula (IX) above.

In a further embodiment of the present invention, the PKI compounds of Formula (IX) have a formula selected from the group of:

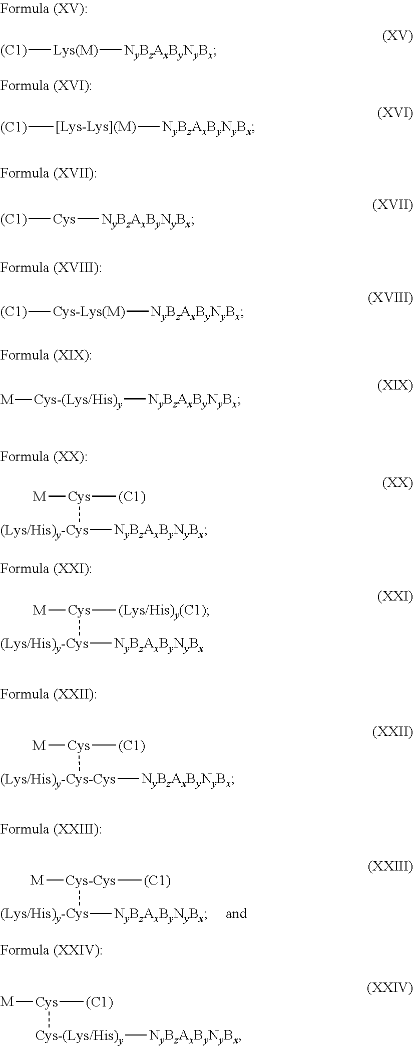

Cys-(Lys/His)_{y}-N_{y}B_{z}AₓB_{y}N_{y}Bₓ

wherein:
--- represents a disulphide bond, and
C1, M, N, A, B, x, y and z are as defined for Formula (IX) above.

In another embodiment of the present invention, the PKI compounds of Formula (IX) have a formula selected from the group of:

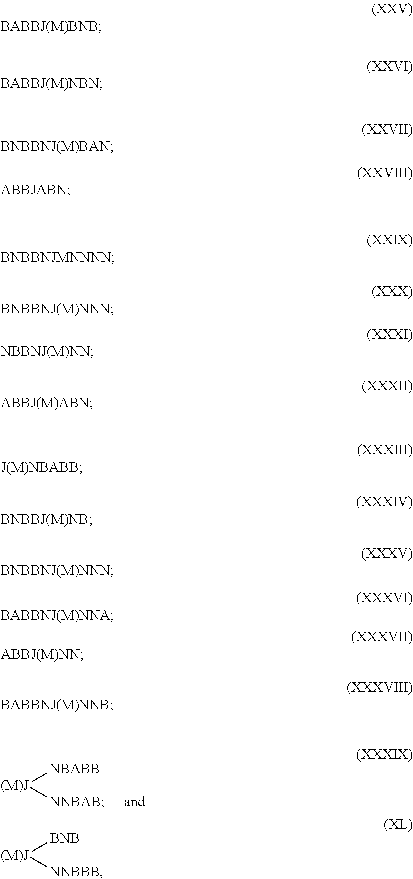

wherein:
M, J, N, A and B are as defined for Formula (IX) above.

In another embodiment of the present invention, the PKI compounds of Formula (IX) comprise between about 5 and about 18 amino acid residues. In a further embodiment, the PKI compounds of Formula (IX) comprise between about 5 and about 16 amino acid residues. In another embodiment, the PKI compounds of Formula (IX) comprise between about 6 and about 20 amino acid residues. In another embodiment, the PKI compounds of Formula (IX) comprise between about 6 and about 18 amino acid residues. In another embodiment, the PKI compounds of Formula (IX) comprise between about 7 and about 20 amino acid residues. In another embodiment, the PKI compounds of Formula (IX) comprise between about 7 and about 18 amino acid residues.

In another embodiment of the present invention, the PKI compounds of Formula (IX) comprise one or more of the amino acid sequences set forth in Table 3.

TABLE 3
Representative PKI Amino Acid Sequences

| Amino Acid Sequence | SEQ ID NO. |
|---|---|
| LRRAKLG | 36 |
| FRRKFRL | 37 |
| HCIGRFK | 38 |
| GCKGKFKR | 39 |
| KFRRKRGR | 40 |
| KFRRKLRL | 41 |
| KLRRAKRFL | 42 |
| FRRCFRL | 43 |
| KLRRAKLGLG | 44 |
| KLKKAKLGL | 45 |
| GCKGKFKR | 46 |
| KAKKKKAK | 47 |
| KLKKLLLVI | 48 |
| RFRKAKKGGH | 49 |
| FRRKLI | 50 |
| KFRKAKKGLK | 51 |
| GCRGR | 52 |
| KKCGGKKK | 53 |
| KFRRKRGREVD | 54 |
| KFRRKLRLEVD | 55 |
| KLRRAKRFLEVD | 56 |
| KLRRAKLGLGDD | 57 |
| KAKKKKAKEGE | 58 |
| RFRKAKKGGHEIE | 59 |
| KFRKAKKGLKEVEK | 60 |
| GCRGREVD | 61 |

PKI compounds contemplated by the present invention include, but are not limited to, the following exemplary compounds:

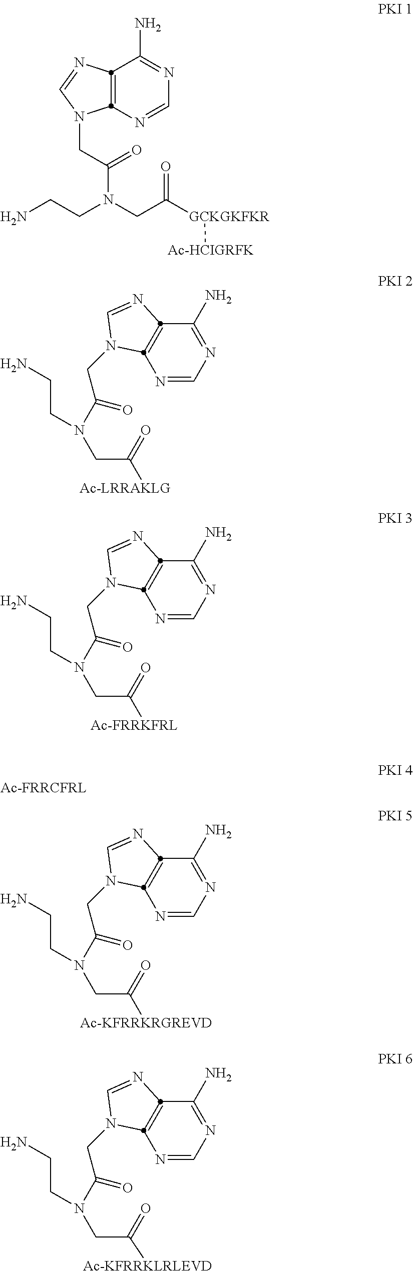

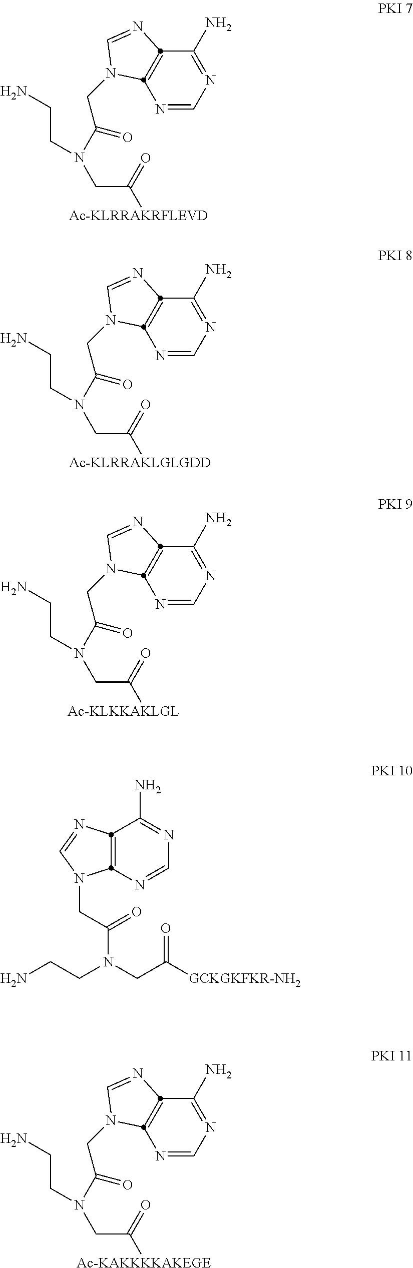

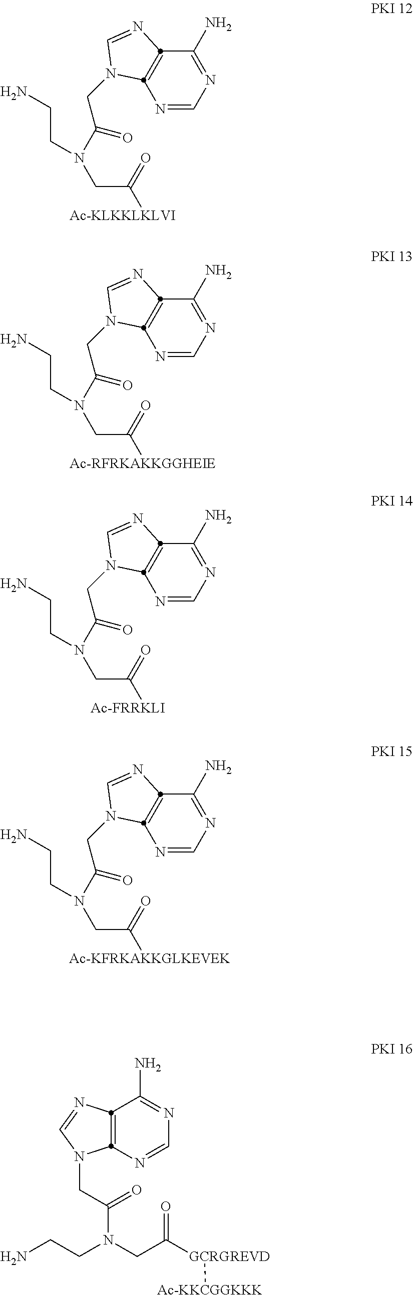

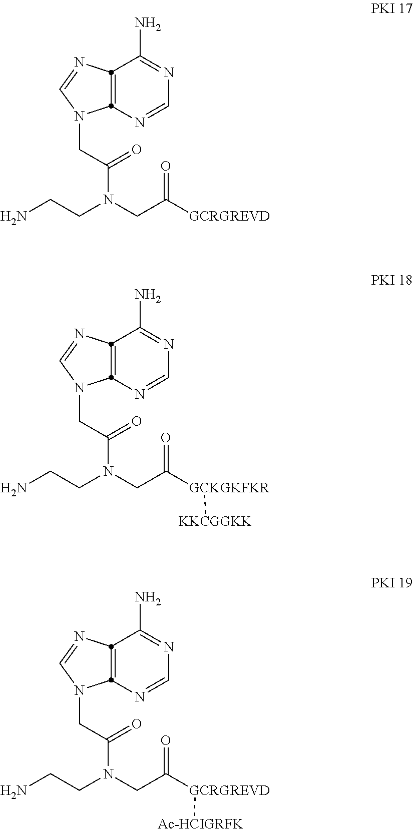

As depicted above in general Formula (I), the PKI compounds can be in the form of a single amino acid chain, or in the form of two cross-linked amino acid chains. In the context of the present invention, an “amino acid chain” is a sequence of amino acid residues linked together by peptide bonds.

The PKI compound can comprise one, or more than one, non-naturally occurring amino acids. When PKI compound comprises more than one non-naturally occurring amino acids, the non-naturally occurring amino acids can be the same or different.

The PKI compound can comprise a free amino-terminus and/or carboxy-terminus, or a modified amino- and/or carboxy-terminus. For example, the N- and/or C-terminus of the PKI compound can be modified to include a chemical substituent group or other chemical modification, a blocking group or additional amino acids. Examples of chemical substituent groups suitable for modifying the amino- and/or carboxy-terminus of peptides are known in the art and examples are provided above.

The presence of extra amino acids to one of the termini of the PKI compound may be desirable, for example, to improve the stability of the final TIM, to incorporate a “tag” to aid in identification, detection or purification protocols, to improve solubility or to improve pharmokinetic parameters. As noted above, in one embodiment of the present invention, the PKI compound is modified at the C-terminus to include a “tag” of between 1 to 4 amino acids in length that comprises one or more acidic amino acid residues. Addition of one or more acidic residues at the C-terminus of the PKI compound can help to improve the interaction of the compound with the target protein kinase. Non-acidic residues included in the tag are selected from the group of: Gly, Val, Ile, Leu and Lys. Examples of suitable tags that can be added at the C-terminal end include, but are not limited to, Glu-Val-Glu; Asp-Asp, Glu-Gly-Glu; Glu-Ile-Glu; Glu-Val-Glu-Lys and Glu-Val-Asp.

In one embodiment of the present invention, the N-terminus of the PKI compound is modified with an acyl group. In another embodiment, the N-terminus is modified with an acetyl group. In another embodiment, the C-terminus is modified with an amino group.

The PKI compound can comprise one, or more than one, non-naturally occurring peptide bonds. When the PKI compound comprises more than one non-naturally occurring peptide bonds, the non-naturally occurring peptide bonds can be the same or different.

As indicated above, the PKI compound can comprise a disulphide bond between two cysteine residues. The present invention also contemplates the use of a suitable chemical groups to cross-link two peptide chains comprised by a PKI compound of Formula (IX). Examples of such chemical groups are well known in the art.

As indicated above, in one embodiment of the present invention, the PKI compounds comprise an ATP mimetic moiety which includes adenine, or a derivative of adenine. A “derivative of adenine,” as used herein, refers to a compound that retains the heteroaromatic ring structure of adenine (shown below) but which may contain additional, fewer or different substituents attached to the ring structure and/or additional, fewer or different heteroatoms within the ring structure when compared to adenine.

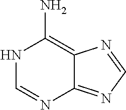

The term “derivative of adenine” also encompasses molecules that are isosteric with adenine. In the context of the present invention, a molecule that is isosteric with adenine (an “adenine isostere”) is a molecule that has a similarity of structure and spatial orientation to adenine and a resulting similarity of properties, in particular with respect to three-dimensional space-filling properties.

Suitable adenine derivatives are known in the art and include, but are not limited to, 1-deazaadenine; 3-deazaadenine; 7-deazaadenine; 7-deaza-8-azaadenine; 1-methyladenine; 2-aminoadenine; 2-propyl and other 2-alkyl derivatives of adenine; 2-aminopropyladenine; 8-amino, 8-aza, 8-thiol, 8-thioalkyl, 8-hydroxyl and other 8-substituted adenines; 8-oxo-N⁶-methyladenine; N⁶-methyladenine; N⁶-isopentenyladenine; 2-aminopurine; 2,6-diaminopurine; 2-amino-6-chloropurine; 6-thio-2-aminopurine; hypoxanthine; inosine; xanthine; 8-aza derivatives of 2-aminopurine, 2,6-diaminopurine, 2-amino-6-chloropurine, hypoxanthine, inosine and xanthine; 7-deaza-8-aza derivatives of 2-aminopurine, 2,6-diaminopurine, 2-amino-6-chloropurine, hypoxanthine, inosine and xanthine; 1-deaza derivatives of 2-aminopurine, 2,6-diaminopurine, 2-amino-6-chloropurine, hypoxanthine, inosine and xanthine; 7-deaza derivatives of 2-aminopurine, 2,6-diaminopurine, 2-amino-6-chloropurine, hypoxanthine, inosine and xanthine; and 3-deaza derivatives of 2-aminopurine, 2,6-diaminopurine, 2-amino-6-chloropurine, hypoxanthine, inosine and xanthine; and adenine isosteres, such as 4-methylindole.

In accordance with one embodiment of the present invention, the ATP mimetic moiety is an adenine peptide nucleic acid (PNA) of the general Formula (XLI):

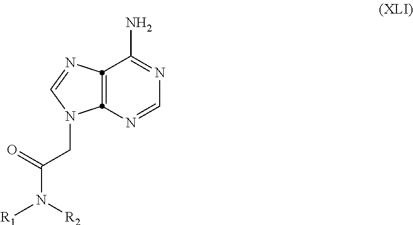

wherein:
R₁ and R₂ are independently alkyl substituted with a carboxyl, carbonyl, alcohol or primary amino (i.e. —COOH, —C(O)R, where R is alkyl or H, —OH or —NH₂).

In one embodiment of the present invention, in Formula (XLI), R₁ is —CH₂CH₂NH₂; and R₂ is —CH₂COOH.

In the PKI compounds of Formula (IX), the ATP moiety (M) when present can be linked to the peptidic moiety using a number of standard linking groups known in the art. In one embodiment of the present invention, the ATP mimetic moiety is attached to the peptidic moiety of the PKI compound via a linking group attached to a nitrogen atom in the heteroaromatic ring structure. Attachment through a substituent amino group, such as N⁶ of adenine, is also contemplated.

In accordance with one embodiment of the present invention, in which the ATP mimetic moiety is provided as an adenine peptide nucleic acid (PNA) of general Formula (XLI), this moiety can be linked to the peptidic moiety by formation of a peptide bond with a N-terminal NH₂ group or a C-terminal CO₂H group of the peptidic moiety, or with an amine group in the side chain of a lysine or arginine residue in the peptidic moiety.

In one embodiment of the present invention, the PKI compounds comprise an adenine PNA of general Formula (XLI) as the ATP mimetic moiety, which is attached to the peptidic moiety by a peptide bond to a N-terminal NH₂ group. In another embodiment, the adenine PNA of general Formula (XLI) is attached to the peptidic moiety by a peptide bond to an amine group in the side chain of a lysine residue.

### III. Spacer

As indicated above, the PRE and inhibitor moiety can be directly connected or they can be indirectly connected via an appropriate spacer.

In the context of the present invention, the spacer acts as a molecular bridge to link the two entities of the TIM (i.e. the inhibitor moiety and the PRE). The spacer can serve, for example, simply as a convenient way to link the two entities, as a means to spatially separate the two entities, to provide an additional functionality to the TIM, or a combination thereof. For example, it may be desirable to spatially separate the inhibitor moiety and the PRE to prevent the PRE interfering with the activity of the inhibitor moiety and/or vice versa. The spacer can also be used to provide, for example, lability to the connection between the two components of the TIM, an enzyme cleavage site, a stability sequence, a molecular tag, a detectable label, a cell permeability enhancer, or various combinations thereof.

In general the selected spacer is bifunctional or polyfunctional, i.e. contains at least a first reactive functionality at, or proximal to, a first end of the spacer that is capable of bonding to, or being modified to bond to, the PRE and a second reactive functionality at, or proximal to, the opposite end of the spacer that is capable of bonding to, or being modified to bond to, the inhibitor molecule of the TIM. The two or more reactive functionalities can be the same (i.e. the spacer is homobifunctional) or they can be different (i.e. the spacer is heterobifunctional). A variety of bifunctional or polyfunctional cross-linking agents are known in the art that are suitable for use as spacers (for example, those commercially available from Pierce Chemical Co., Rockford, Ill.). Alternatively, these reagents can be used to link the spacer to the PRE and/or inhibitor moiety.

The length and composition of the spacer can be varied considerably provided that it can fulfil its purpose as a molecular bridge. The length and composition of the spacer are generally selected taking into consideration the intended function of the spacer, and optionally other factors such as ease of synthesis, stability, resistance to certain chemical and/or temperature parameters, and biocompatibility. For example, the spacer should not significantly interfere with the ability of the PRE to target PKC or with the inhibitory activity of the inhibitor moiety.

In one embodiment of the present invention, the composition and length of the spacer are selected to provide a flexible spacer. In another embodiment, the composition of the spacer is selected to provide a non-planar spacer.

In accordance with one embodiment of the present invention, the spacer is a branched or unbranched, saturated or unsaturated, hydrocarbon chain having from 1 to 100 carbon atoms, wherein one or more of the carbon atoms is optionally replaced by —O— or —NR— (wherein R is H, or C1 to C6 alkyl), and wherein the chain is optionally substituted on carbon with one or more substituents selected from the group of (C1-C6) alkoxy, (C3-C6) cycloalkyl, (C1-C6) alkanoyl, (C1-C6) alkanoyloxy, (C1-C6) alkoxycarbonyl, (C1-C6) alkylthio, amide, azido, cyano, nitro, halo, hydroxy, oxo (═O), carboxy, aryl, aryloxy, heteroaryl, and heteroaryloxy.

Examples of suitable spacers include, but are not limited to, peptides having a chain length of 1 to 100 atoms, and spacers derived from groups such as ethanolamine, ethylene glycol and polyethylene with a chain length of 6 to 100 carbon atoms, polyethylene glycol with 3 to 30 repeating units, phenoxyethanol, propanolamide, butylene glycol, butyleneglycolamide, propyl phenyl, and ethyl, propyl, hexyl, steryl, cetyl, and palmitoyl alkyl chains. Other examples include spacers based on 1,3-diamino propane or ethane.

In one embodiment, the spacer is a branched or unbranched, saturated or unsaturated, hydrocarbon chain, having from 1 to 50 carbon atoms, wherein one or more of the carbon atoms is optionally replaced by —O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (═O), carboxy, aryl and aryloxy.

In another embodiment, the spacer is an unbranched, saturated hydrocarbon chain having from 1 to 50 carbon atoms, wherein one or more of the carbon atoms is optionally replaced by —O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (═O), carboxy, aryl and aryloxy.

In a specific embodiment of the present invention, the spacer comprises spacers 1,3-diamino propane or ethane, or is a peptide having a chain length of 1 to 50 atoms. In another embodiment, the spacer is a peptide having a chain length of 1 to 40 atoms.

In an alternate embodiment, the spacer is a peptide of between about 1 to about 20 amino acid residues. In another embodiment, the spacer is a peptide of between about 1 to about 18 amino acid residues. In a further embodiment, the spacer is a peptide of between about 1 to about 16 amino acid residues. In other embodiments, the spacer is a peptide of between about 1 to about 15 amino acid residues, between about 1 and about 14, between about 1 and about 12 and between about 1 and about 10. In another embodiment, the spacer is a peptide comprising amino acids selected from the group of glycine, alanine, valine, lysine and isoleucine. In another embodiment, the spacer is a peptide comprising amino acids selected from the group of glycine, alanine, valine and isoleucine. In another embodiment, the spacer is a polyglycine peptide.

### IV. Other Components

The present invention contemplates that the TIMs may further comprise one or more additional components. The additional component(s) can be conjugated to an appropriate reactive functionality on the PRE, on the inhibitor molecule, on the spacer, or a combination thereof. The additional components can act to stabilise the TIM, provide an additional targeting functionality, provide a detectable label, facilitate preparation, isolation and/or purification of the TIM, increase bioavailability of the TIM, improve the pharmacokinetics of the TIM, and the like.

Thus, for example, the TIM can be conjugated to one or more of a protein, peptide or carrier, a lipophilic moiety (for example, octyl, caproyl, lauryl, stearoyl moieties), an antibody or other biological ligand, a detectable label, a cell permeability enhancer, a moiety that provides additional targeting properties, a moiety that enhances bioavailability, biodistribution, and/or stability of the TIM, a moiety that facilitates preparation, isolation and/or purification of the TIM, or a moiety that improves the physiological half-life of the TIM. The TIM can also be glycosylated or phosphoylated.

Examples of detectable labels that can be conjugated to the TIM include, for example, radioisotopes, fluorophores, chemiluminophores, colloidal particles, fluorescent microparticles, chromophores, fluorescent semiconductor nanocrystals, enzyme substrates, enzyme cofactors, enzyme inhibitors, dyes, metal ions, metal sols, ligands (such as biotin, strepavidin or haptens), and the like. One skilled in the art will understand that these labels may require additional components, such as triggering reagents, light, binding partners, and the like to enable detection of the label.

Examples of cell permeability enhancers that can be conjugated to the TIM include, but are not limited to, the penetratin peptide derived from the Drosophila antennapedia protein (RQIKIWFQNRRMKWKK; also available in activated form as Penetratin™ 1 Peptide from Qbiogene, Inc., Irvine, Calif.); the cell-penetrating region of the HIV tat protein (amino acid 47-57: RRRQRRKKR) (see, Vives, E. & Lebleu, B. (2002) in Cell-Penetrating Peptides, ed. Langel, U. (CRC, Boca Raton, Fla.), Vol. 1, pp. 3-23); the Protein Transport Domain, a sequence derived from the HIV virus (KRRQRRKKR; Fuchs and Raines, 2003, Biochemistry, 43:2438-44); the Fc peptide (YGRKKRRQR; Kim D, et al. (2006) Experimental Cell Research, 312:1277-1288); Transport™ (Cambrex BioScience Inc., Baltimore, Md.) and BioTrek™ (Stratagene, La Jolla, Calif.).

Additional targeting properties can be provided by conjugation of the TIM to cell targeting compounds, for example, the Ricin B chain or modifications thereof, portions of peptides that mediate virus-cell fusion such as DP178, and small chemokines such as SDF-1 and RANTES.

Moieties that facilitate preparation, isolation and/or purification of the TIM include, for example, His-tags, biotin, streptavidin, glutathione-S-transferase (GST), and the like.

The present invention also contemplates that further modifications can be made to the TIM in order to enhance one or more of the properties of the compound as described above. For example, one or more of the amino acids in the TIM can be esterified, pegylated, acetylated and/or amidated.

One skilled in the art will understand that the other components for conjugation to the TIM should be selected such that they do not interfere with the ability of the TIM to target and inhibit its target PKC.

In a specific embodiment of the present invention, the TIM comprises a PKI compound of general formula (IX) operatively associated by way of a spacer with a PRE of general formula (I). In another embodiment, the TIM of the present invention comprises a PKI compound that comprises an amino acid sequence as set forth in any one of SEQ ID NOs: 36, 37, 38, 39, 40, 41, 42, 43, 44, 45, 46, 47, 48, 49, 50, 51, 52, 53, 54, 55, 56, 57, 58, 59, 60, 61, 62 or 63 linked via a spacer to a PRE of general formula (I). In a further embodiment, the TIM comprises a PKI compound sekected from the groups of: compound PKI 1, compound PKI 2, compound PKI 3, compound PKI 4, compound PKI 5, compound PKI 6, compound PKI 7, compound PKI 8, compound PKI 9, compound PKI 10, compound PKI 11, compound PKI 12, compound PKI 13, compound PKI 14, compound PKI 15, compound PKI 16, compound PKI 17, compound PKI 18 and compound PKI 19 linked by means of a spacer to a PRE of general formula (I). In a further embodiment, the PRE is less than about 30 amino acid residues in length that comprises an amino acid sequence selected from the group of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 and SEQ ID NO:35, or the retro, inverso, or retro-inverso form thereof, wherein each of the N-terminus and C-terminus of the PRE are independently either free or modified.

In a further embodiment, the TIM is designed to preferentially target one or a subset of PKC isoforms. As demonstrated in the Examples provided herein, all the PREs all target at least one PKC isoform, and some show specificity for certain isoforms. For example, PRE 2 shows specificity towards PKC-δ, and PRE 4 shows specificity towards PKC-α.

In another embodiment, the TIM comprises one of the following combinations of PRE and PKI compounds: PRE 11 and PKI 3; PRE 4 and PKI 10; PRE 4 and PKI 3; PRE 10 and PKI 3; PRE 3 and PKI 4; PRE 3 and PKI 9; PRE 1 and PKI 4; PRE 4 and PKI 1; PRE 4 and PKI 3; PRE 4 and PKI 4; PRE 4 and PKI 5.

The PKI compound can be conjugated to the PRE at the N- or C-terminus either directly or via a spacer. In one embodiment, the PRE and PM components noted above are conjugated via a spacer. In another embodiment, the PRE and PKI components are conjugated via a peptide spacer. One or more of the amino acids in the PRE or PKI compound can be modified. Similarly the N- and/or C-terminus of either or both components can be modified. The TIM can further comprise an additional component as described above.

Representative non-limiting examples of the TIMs of the present invention include those shown in Table 28 in the Examples provided below.

### Preparation of the Targeted Inhibitory Molecules

The TIMs of the present invention can be prepared using standard synthetic techniques known in the art. The components of the TIM can be prepared sequentially, concurrently or as part of a single process. For example, the PRE molecule can be synthesized and then conjugated using standard conjugation chemistry techniques to the inhibitor molecule, which can either have been synthesized separately or obtained from commercial sources. Alternatively, the PRE and the inhibitor molecule can be synthesized together as a single molecule. Similarly, when a spacer is present, the spacer can be synthesized together with the PRE and/or the inhibitor molecule, or it can be synthesized separately, or obtained from commercial sources, and conjugated to the PRE and inhibitor moiety sequentially or in a single reaction.

For example, when the inhibitor moiety is a peptidic compound and is either directly connected to the PRE or connected via a peptidic spacer, the TIM can be synthesized sequentially or as a single molecule. Similarly, when a peptidic spacer is employed and a non-peptidic inhibitor moiety, the PRE and spacer can be synthesized as a single molecule and then conjugated to the inhibitor moiety.

As indicated above, many protein kinase inhibitors suitable for incorporation into the TIMs of the present invention can be obtained from commercial sources (for example, from Biaffin GmbH & Co KG, Kassel, Germany; EMD Biosciences, San Diego, Calif., and Sigma-Aldrich, St. Louis, Mo.), as can many bifunctional cross-linking agents suitable for incorporation into the TIMs as spacers (for example, from Pierce Chemical Co., Rockford, Ill. and Sigma-Aldrich, St. Louis, Mo.).

Peptidic components of the TIM, i.e. the PREs, peptidic inhibitor moieties, such as the PKI compounds described above, and peptidic spacers, and combinations of these peptidic components, can be readily prepared by standard peptide synthesis techniques known in the art, for example, by standard solution, suspension or solid phase techniques, such as exclusive solid phase synthesis, partial solid phase synthesis methods, fragment condensation and classical solution synthesis.

In one embodiment of the present invention, solid phase techniques are employed to prepare peptidic components of the TIMs. The principles of solid phase chemical synthesis of peptides are well known in the art and may be found in general texts in the area such as Pennington, M. W. and Dunn, B. M., Methods in Molecular Biology, Vol. 35 (Humana Press, 1994); Dugas, H. and Penney, C., Bioorganic Chemistry (1981) Springer-Verlag, New York, pgs. 54-92; Merrifield, J. M., Chem. Soc., 85:2149 (1962), and Stewart and Young, Solid Phase Peptide Synthesis, pp. 24-66, Freeman (San Francisco, 1969).

An insoluble polymer support (or resin) is used to prepare the starting material by attaching a protected version of the required α-amino acid to the resin. The resin acts to anchor the peptide chain as each additional α-amino acid is attached and is composed of particles (generally between about 20-50 μm diameter) that are chemically inert to the reagents and solvents used in solid phase peptide synthesis. These particles swell extensively in solvents, which makes the linker arms more accessible. Examples of resins used in solid phase peptide synthesis include chloromethylated resins, hydroxymethyl resins, benzhydrylamine resins, and the like. Various resins suitable for solid phase peptide synthesis applications are available commercially, for example, phenylacetamidomethyl (PAM) resin, hydroxymethyl polystyrene-vinylbenzene copolymer, polyamide, p-benzyloxybenzyl alcohol resin (Wang resin) and modified versions thereof, 4-hydroxymethylphenoxymethyl-copoly(styrene-1% divinylbenzene), and 4-(2′,4′-dimethoxyphenyl-Fmoc-aminomethyl)phenoxyacetamidoethyl and [5-(4-Fmoc-aminomethyl-3,5-dimethoxyphenoxy)valeric acid]-polyethylene glycol-polystyrene resins (which are commercially available from Applied Biosystems, Foster City, Calif.) and can be used in the preparation of the peptidic components of the TIMs of the invention.

The α-amino acid is coupled to the resin using a standard coupling reagent such as N,N′-dicyclohexylcarbodiimide (DCC), N,N′-diisopropylcarbodiimide (DIC) or O-benzotriazol-1-yl-N,N,N′,N′-tetramethyluronium-hexafluorophosphate (HBTU), with or without 4-dimethylaminopyridine (DMAP), 1-hydroxybenzotriazole (HOBT), benzotriazol-1-yloxy-tris(dimethylamino)phosphonium-hexafluorophosphate (BOP) or bis(2-oxo-3-oxazolidinyl)phosphine chloride (BOPCl). The coupling generally takes place in a solvent such as dichloromethane, DMF or NMP.

After the initial coupling, the α-amino protecting group is removed using a standard reagent, such as a solution of trifluoroacetic acid (TFA), hydrochloric acid in an organic solvent or 20% piperidine in DMF solvent.

Suitable α-amino protecting groups are known in the art of and include, for example, acyl type protecting groups (such as, formyl, trifluoroacetyl, acetyl), aromatic urethane type protecting groups (such as, benzyloxycarboyl (Cbz) and substituted Cbz), aliphatic urethane protecting groups (such as, t-butyloxycarbonyl (Boc), isopropyloxycarbonyl and cyclohexyloxycarbonyl), alkyl type protecting groups (such as, benzyl and triphenylmethyl) and 9-fluorenylmethoxy carbonyl (Fmoc). A labile group protects the alpha-amino group of the amino acid. This group should be easily removed after each coupling reaction so that the next α-amino protected amino acid may be added.

Side chain protecting groups, when used, remain intact during coupling and typically are not removed during the deprotection of the amino-terminus protecting group or during coupling. Side chain protecting groups are generally selected such that they are removable upon the completion of the synthesis of the final peptide and under reaction conditions that will not alter the peptide. Examples of side chain protecting groups include, but are not limited to, benzyl, 2,6-dichlorobenzyl, methyl, ethyl, and cyclohexyl for Asp; acetyl, benzoyl, trityl, tetrahydropyranyl, benzyl, 2,6-dichlorobenzyl, and Cbz for Ser; nitro, Tosyl (Tos), Cbz, adamantyloxycarbonyl mesitoylsulfonyl (Mts), or Boc for Arg and Cbz, 2-chlorobenzyloxycarbonyl (2-C1-Cbz), and 2-bromobenzyloxycarbonyl (2-BrCbz), ivDde, Tos, or Boc for Lys. Other examples are known in the art.

After removal of the α-amino protecting group, the remaining protected amino acids are coupled in the desired order to the peptide chain in a stepwise manner. An excess of each protected amino acid is generally used with an appropriate carboxyl group activator, such as dicyclohexylcarbodiimide (DCC) in methylene chloride and/or dimethyl formamide (DMF), N-[(dimethylamino)-1H-1,2,3-triazolo[4,5-b]pyridin-1-ylmethylene]-N-methylmethanaminium hexafluorophosphate N-oxide (HATU), N-[1H-benzotriazol-1-yl)-(dimethylamino)methylene]-N-methylmethanaminium hexafluorophosphate N-oxide (HBTU), and (benzotriazol-1-yl-N-oxy)tris(dimethylamino)phosphonium hexafluorophosphate (BOP).

Once the desired amino acid sequence has been synthesized, the stable blocking groups are removed and the peptide is decoupled from the resin support by treatment with a suitable reagent, such as Reagent K, which includes TFA (82.5%), Thioanisole (5%), Phenol (5%), H₂O (5%), 1,2-ethanedithiol (EDT, 2.5%). The decoupling reagent may simultaneously cleave any side chain protecting groups. Alternatively, the side chain protecting groups can be cleaved off using a separate reagent, for example, 20% piperidine in DMF for Fmoc groups or 2% hydrazine in DMF for ivDde groups.

In one embodiment of the present invention, peptidic components of the TIMs are synthesized on a commercially available peptide synthesizer (such as the Pioneer Peptide Synthesizer available from Applied Biosystems, Foster City, Calif., or the Liberty System from CEM Corporation, Matthews, N.C.) following the manufacturer's instructions and employing suitable protecting groups to protect the amino acid side chains, as necessary.

The above techniques can also be used to synthesize peptidic components of the TIM which include one or more non-naturally occurring amino acids. Covalent modifications can be introduced, for example, by reacting targeted amino acid residues with an organic derivatising agent that is capable of reacting with selected amino acid side chains or with the terminal residue(s) as is known in the art. Selection of appropriate derivatising agent(s) can be readily accomplished by a worker skilled in the art.

Methods of synthesizing peptides having one or more modified peptide bonds are known in the art (see, for example, “Solid Phase Peptide Synthesis” Methods in Enzymology (ed. Fields, G. B. (1997) Academic Press, San Diego).

The peptidic components of the TIM can also be prepared in their salt form. The peptides may be sufficiently acidic or sufficiently basic to react with a number of inorganic bases, inorganic acids or organic acids, to form a salt. Acids commonly employed to form acid addition salts are inorganic acids such as hydrochloric acid, hydrobromic acid, hydroiodic acid, sulphuric acid, phosphoric acid, and the like, and organic acids such as p-toluenesulphonic acid, methanesulphonic acid, oxalic acid, p-bromophenyl-sulphonic acid, carbonic acid, succinic acid, citric acid, benzoic acid, acetic acid, and the like.

Base addition salts include those derived from inorganic bases, such as ammonium or alkali or alkaline earth metal hydroxides, carbonates, bicarbonates, and the like. Examples of bases useful in preparing the salts include, but are not limited to, sodium hydroxide, potassium hydroxide, ammonium hydroxide, potassium carbonate, and the like.

The present invention also contemplates that when the peptidic components of the TIM comprise naturally occurring amino acids or slightly modified versions thereof, they can be prepared by recombinant DNA techniques. Such methods can be found generally described in Ausubel et al. (Current Protocols in Molecular Biology, Wiley & Sons, NY (1997 and updates)) and Sambrook et al. (Molecular Cloning: A Laboratory Manual, Cold-Spring Harbor Press, NY (2001)). In general, a DNA sequence encoding the peptidic component is prepared and inserted into a suitable expression vector. The expression vector is subsequently introduced into a suitable host cell or tissue by one of a variety of methods known in the art, for example, by stable or transient transfection, lipofection, electroporation, or infection with a recombinant viral vector. The host cell or tissue is cultured under conditions that allow for the expression of the peptidic component and the peptidic component is subsequently isolated from the cells/tissue.

Examples of suitable expression vectors include, but are not limited to, plasmids, phagemids, cosmids, bacteriophages, baculoviruses and retroviruses, and DNA viruses. The selected expression vector can further include one or more regulatory elements to facilitate expression of the peptidic component, for example, promoters, enhancers, terminators, and polyadenylation signals. One skilled in the art will appreciate that such regulatory elements may be derived from a variety of sources, including bacterial, fungal, viral, mammalian or insect genes.

In the context of the present invention, the expression vector may additionally contain heterologous nucleic acid sequences that facilitate the purification of the expressed peptidic component. Examples of such heterologous nucleic acid sequences include, but are not limited to, affinity tags such as metal-affinity tags, histidine tags, avidin/strepavidin encoding sequences, glutathione-S-transferase (GST) encoding sequences and biotin encoding sequences.

One skilled in the art will understand that selection of the appropriate host cell for expression of the recombinant peptidic component will be dependent upon the vector chosen. Examples of suitable host cells include, but are not limited to, bacterial, yeast, insect, plant and mammalian cells.

If the peptidic components of the TIM cannot be encoded or expressed but are very similar to a peptide that can be encoded or expressed, genetic engineering techniques such as those described above can be employed to prepare the encodable peptide, followed by one or more steps in which the encoded peptide is modified by chemical or enzymatic techniques to prepare the final peptidic component.

Standard conjugation techniques known in the art can be employed to conjugate the individual components of the TIM together, where necessary, and/or to conjugate the TIM to one or more additional components, such as those described above (see, for example, Morrison and Boyd, Organic Chemistry, 6th Ed. (Prentice Hall, 1992); J. March, Advanced Organic Chemistry, 4^{th} Ed. (Wiley 1992); G. T. Harmanson, Bioconjugate Techniques, (Academic Press, Inc. 1995), and S. S. Wong, Chemistry of Protein Conjugation and Cross-Linking, (CRC Press, Inc. 1991)).

The components are conjugated through a reactive functionality on one or more of the components either directly or by modification of the group to introduce a new chemical group capable of conjugating a second component. A variety of chemical groups can be subject to conjugation reactions. For example, hydroxyl groups (—OH) can be used to conjugate a second component through reaction with alkyl halides (R—Cl, R—Br), acyl anhydrides, acyl halides, aldehydes (—CHO), hydrazides (R—CO—NH—NH₂), and the like. Primary amino groups (—NH₂) can be used to conjugate a second component through reaction with alkyl halides (R—Cl, R—Br, R—I), aryl azides, acyl anhydrides, acyl halides, acyl esters, carboxylates activated with carbodiimides, aldehydes (—CHO), and the like. Carboxylic groups (—COOH) can also be used to conjugate a second component after the group has been activated. Suitable activation agents include, for example, organic or inorganic acid halides (for example pivaloyl chloride, ethyl chloroformate, thionyl chloride, PCl₅), carbodiimides (R—CO—OH+R′—N═C═N—R″, for example EDC, DCC), benzotriazolyl uronium or phosphonium salts (TBTU, BOP, PyBOP, HTBU), diacyl chlorides, diisocyanates, and the like.

Some of the above reagents can also be used as bifunctional cross-linking reagents that can be employed to conjugate the components of the TIM. A variety of such cross-linking reagents is known in the art and many are commercially available (see, for example, S. S. Wong, ibid., and catalogues from Pierce Chemical Co. and Sigma-Aldrich). Examples include, but are not limited to, diamines, such as 1,6-diaminohexane; dialdehydes, such as glutaraldehyde; bis-N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyantes, such as hexamethylenediisocyanate; bis oxiranes, such as 1,4 butanediyl diglycidyl ether; dicarboxylic acids, such as succinyldisalicylate; 3-maleimidopropionic acid N-hydroxysuccinimide ester, and the like.

Prior to conjugation, one or more of the components of the TIM can be submitted to one or more purification procedures, as can the final TIM. Purification methods are well known in the art (see, for example, T. Hanai, HPLC: A Practical Guide, RSC Press, UK 1999; L. M. Harwood, C. J. Moody and J. M. Percy, Experimental Organic Chemistry: Standard and Microscale, Blackwell Scientific Publishing, 1998; Current Protocols in Protein Science, Coligan, J. E., et al. (eds.), John Wiley & Sons, (2001 & updates)) and can include one or more chromatographic steps, for example, ion exchange chromatography, hydrophobic adsorption/interaction chromatography, silica gel adsorption chromatography, and various forms of high performance liquid chromatography (HPLC), such as reverse-phase HPLC.

### Activity of the Targeted Inhibitory Molecules

In accordance with the present invention, the TIMs are capable of targeting and inhibiting the activity of one or more PKC isoform and of modulating one or more PKC-mediated physiological effects. A candidate TIM can be tested for the above activities in vitro and/or in vivo using a number of standard techniques known in the art. Exemplary assays are described below and in the Examples provided herein. Similarly, when preparing a TIM specifically targeted to one PKC isoform or a sub-group of isoforms, the affinity of the selected PRE component of the TIM can be assessed initially using standard techniques such as those described below.

### I. Affinity and Binding Assays

In accordance with the present invention, the PRE incorporated into the TIM of the present invention has an affinity for one or more PKC isoform and, as such, is able to target the TIM to the PKC isoform(s). As noted above, the term “affinity” means that the TIM or PRE is capable of interfering with the binding of a PKC-isoform specific antibody to its target isoform. The affinity of the PREs and the TIMs for PKC can be tested using one or more of a number of standard assay techniques known in the art.

Typically, the ability of a candidate PRE or TIM to interfere with the binding of a PKC isoform-specific antibody to PKC-α is tested in a competitive binding assay, in which the candidate PRE/TIM and a PKC isoform-specific antibody are combined with the PKC and the extent to which the PRE/TIM decreases binding of the antibody to the PKC is determined by comparison with a control assay conducted in the absence of the PRE/TIM. The extent to which the PRE/TIM has decreased binding of the antibody to the PKC in the assay can be determined for example, by quantifying the amount of protein:antibody complex that has formed in the assay and comparing this to the amount of protein:antibody complex that has formed in the control assay. The PKC can be provided in the assay as a purified or partially purified protein, or it may be provided as a crude or partially purified cell extract or as a cell lysate.

The anti-PKC antibody can be labelled with a detectable label in order to facilitate detection and/or quantitation of the protein:antibody complexes. Alternatively, the anti-PKC antibody (primary antibody) can be detected using a labelled secondary antibody that specifically recognises the primary antibody. If necessary, the protein:antibody complexes can be separated from free PKC (and other reagents, as required) prior to detection and/or quantification. Examples of suitable separation techniques are known in the art and include, for example, filtration, polyacrylamide gel electrophoresis, differential centrifugation, size exclusion chromatography, and the like.

Detectable labels are moieties having a property or characteristic that can be detected directly or indirectly. One skilled in the art will appreciate that when a detectable label is employed, it is selected such that it does not affect the affinity of the antibody for its target PKC. Examples of suitable labels include, but are not limited to, radioisotopes, fluorophores, chemiluminophores, colloidal particles, fluorescent microparticles, chromophores, fluorescent semiconductor nanocrystals, enzymes, enzyme substrates, enzyme cofactors, enzyme inhibitors, dyes, metal ions, metal sols, ligands (such as biotin, strepavidin or haptens), and the like. One skilled in the art will understand that these labels may require additional components, such as triggering reagents, light, binding partners, and the like to enable detection of the label.

Indirectly detectable labels are typically binding elements that are used in conjunction with a “conjugate” that in turn is attached or coupled to a directly detectable label. The binding element and the conjugate represent two members of a “binding pair,” of which one component, the binding element, binds specifically to the target molecule (PRE/TIM, target PKC or primary antibody) and the other of which, the conjugate, specifically binds to the binding element allowing its detection. Binding between the two members of the pair is typically chemical or physical in nature. Examples of such binding pairs include, but are not limited to, antigen/hapten and antibody; antibody and anti-antibody; receptor and ligand; enzyme/enzyme fragment and substrate/substrate analogue/ligand; biotin/lectin and avidin/streptavidin; lectin and carbohydrate; digoxin and anti-digoxin; His-tags and Ni²⁺ ions; benzamidine and trypsin or other serine proteases; protein A and immunoglobulin; pairs of leucine zipper motifs (see, for example, U.S. Pat. No. 5,643,731), bacitracin and undecaphosphoprenyl pyrophosphate as well as various homodimers and heterodimers known in the art.

In one embodiment of the present invention, the ability of candidate PRE/TIM to interfere with the binding of a PKC isoform-specific antibody to its target PKC is tested using the following general method. Cell lysates are obtained from an appropriate cell line using standard protocols. The proteins of the extract are separated by gel electrophoresis and immobilized on a suitable membrane by Western blotting. The membrane is then blocked using an appropriate blocking buffer to which varying concentrations of the candidate PRE/TIM have been added. A primary PKC isoform-specific antibody is then added under conditions that permit binding of the primary antibody to its target PKC and is subsequently detected by standard procedures using a suitable secondary antibody conjugate.

In another embodiment of the present invention, the candidate PRE/TIM is screened by adding various concentrations of the PRE/TIM directly to the cell extract prior to separating the proteins of the extract by gel electrophoresis and Western blotting as described above.

As described above, in one embodiment of the present invention, the PRE/TIM has an affinity for PKC-α and optionally one or more other PKC isoforms. A PRE/TIM of the present invention is considered to be PKC-α specific if it has a greater affinity for PKC-α than for other PKC isoforms, when the affinity for each isoform is tested under the same conditions (i.e. under the same general assay procedure using the same concentration of PRE/TIM).

In accordance with one embodiment of the present invention, the PRE binds to their target PKC isoform(s). The ability of a candidate PRE, or the TIM comprising the PRE, to bind to a PKC can be determined by standard binding assays known in the art. In general these assays involve combining the candidate compound and the target PKC under conditions that permit formation of a peptide:protein complex and then detecting the presence of any complexes as an indication of candidate compound binding to the PKC. As is the case for the affinity assays described above, the PKC can be provided in the binding assay as a purified or partially purified protein, or it may be provided as a crude or partially purified cell extract or as a cell lysate.

Either the candidate PRE/TIM or the PKC can be labelled with a detectable label in order to facilitate detection of the peptide:protein complexes. If necessary, the complexes can be separated from free PRE/TIM and PKC (and other reagents, as required) prior to detection. Examples of suitable separation techniques are known in the art and include those indicated above. Suitable detectable labels are also described above. One skilled in the art will appreciate that the detectable label is chosen such that it does not affect the binding of the PRE/TIM for PKC.

Various techniques for the detection of protein:peptide complexes are known in the art and can be employed in the screening assays of the present invention (see, for example, Current Protocols in Protein Science, Coligan, J. E., et al. (eds.), John Wiley & Sons, (2005 & updates)). Examples include, but are not limited to, polyacrylamide gel electrophoresis, differential centrifugation, size exclusion chromatography, fluorescence polarisation spectrometry, scintillation proximity assay (SPA, which utilises scintillant incorporated into microspheres), Western analysis, Far-Western analysis, equilibrium sedimentation centrifugation (SEC), SEC with on-line light scattering, sedimentation velocity ultracentrifugation, surface plasmon resonance (SPR; for example, using BIACORE® technology; Biacore International AB, Uppsala, Sweden), and chemical cross-linking.

In one embodiment of the present invention, the binding between the candidate PRE/TIM and the PKC is determined by attaching the candidate PRE/TIM to magnetic beads, for example via a biotin-streptavidin binding pair, and then contacting the PRE/TIM with a solution or cell extract containing the PKC. After the beads have been incubated for an appropriate time with the solution/cell extract, the beads are separated from the other components of the assay, for example, by centrifugation or filtration. The separated beads are treated with an appropriate reagent to release any PRE/TIM-PKC complexes from the beads and the released complexes are then detected by Western blotting using an anti-PKC antibody.

In another embodiment of the present invention, the binding between the candidate PRE/TIM and the target PKC is determined by competition binding. PKCs are immunoprecipitated from cell extracts containing PKC, for example, using ProteinA/G-plus agarose beads (from Santa Cruz Biotechnology Inc.). The PKCs are separated by electrophoresis and transferred onto appropriate membranes via electrotransfer. Increasing concentrations of PRE/TIM are applied to separate membranes together with a fixed concentration of specific anti-PKC primary antibody. The PKC bands are detected with an alkaline phosphatase conjugated secondary antibody and the density of the band measured by densitometry scanning. The relative band density of the PKC isoform bands decreases by binding with PRE/TIM due to competition with the primary antibody. The results are expressed as percentage of the band density of controls untreated (no PRE/TIM), i.e. relative intensity. The decrease in relative intensity correlates to the amount of binding of the PRE/TIM to the PKC isoform.

The PKC used in the above affinity and binding screening assays can be a purified or partially purified protein (either native or recombinant), or it can be in the form of a crude or partially purified cell extract or a cell lysate. Suitable purified PKC proteins derived from a variety of sources (including human) and various recombinant PKC-α proteins are available commercially (for example, from Sigma-Aldrich, Mo.; Merck Biosciences GmBH, Germany; Cell Sciences, Inc., MA; Oxford Biomedical Research, Inc., MI, and Tebu-bio SA, France). Alternatively, PKC can be isolated from an appropriate source using standard methodology (see, for example, Dianoux, A. C., et al., (1989) Biochemistry 28:424-431; Greene, N. M., et al., (1995) J. Biol. Chem. 270:6710-6717 Ohguro, H., et al., (1996) J. Biol. Chem. 271:5215-5224 and Huang, K.-P., et al., (1986) J. Biol. Chem. 261:12134-12140).

PKCs are present in almost all cells, therefore, extracts from or lysates of a variety of different cell types can be used as a source of PKCs in the above assays. For example, as is known in the art, PKC-α is overexpressed in a number of different cancers, and cancer cell extracts and or lysates are thus also examples of suitable sources for PKC-α. Other examples of suitable cells include, but are not limited to, neuroblastoma cells, glioma cells, oestrogen-receptor negative breast cancer cells and non-small cell lung cancer cells. Cancer cells are also appropriate sources for other PKC isoforms. For example, lung cancer cells, breast cancer cells, colon cancer cells, prostate cancer cells and bladder cancer cells can be used as a source for PKC-βI, PKC-βII, PKC-δ, PKC-ε, PKC-ι and PKC-ζ. Neuroblastoma, mesangial, promyelocytic leukemia and pancreatic neoplasm cells can also be used as a source of PKC-βI, as well as malignant lymphoma tumour, proximal pancreatic duct and dendritic cells for PKC-βII; endothelial cells and colon cancer cells for PKC-δ; neuroblastoma, upper airway, pancreatic duct and primary gastric tumour cells for PKC-ε; ovarian cancer, non small cell lung cancer and breast cancer cells for PKC-ι; and fibroblasts, immature CD34 monocytes and adipocytes for PKC-ζ.

The specific anti-PKC antibody employed in the above assays can be a polyclonal or a monoclonal antibody. Various anti-PKC antibodies are commercially available (for example, from Sigma-Aldrich, Mo., Oxford Biomedical Research, Inc., MI and Santa Cruz Biotechnology, Inc., CA).

A variety of other reagents may be included in the screening assays. For example, reagents that facilitate optimal protein-antibody, antibody-antibody and/or protein-peptide interactions, reduce non-specific or background interactions and/or otherwise improve the efficiency of the assay can be included. Non-limiting examples of such reagents include, but are not limited to, buffers; salts; neutral blocking proteins, such as albumin; detergents; protease inhibitors; phosphatase inhibitors; nuclease inhibitors; anti-microbial agents, and the like.

The screening assays can be carried out in solution or can be carried out in or on a solid support, or can employ some combination of solution and solid phases. For example, one or more of the components (such as the candidate PRE/TIM, target PKC, primary antibody, or one of the members of a binding pair) can be immobilised on a solid support. Examples of suitable solid supports are known in the art (see, for example, Current Protocols in Protein Science, Coligan, J. E., et al. (eds.), John Wiley & Sons, (2005 & updates); Affinity Chromatography: Principles & Methods, Pharmacia LKB Biotechnology (1988), and Doonan, Protein Purcation Protocols, The Humana Press (1996)). Examples include, but are not limited to, various resins and gels (such as silica-based resins/gels, cellulosic resins/gels, cross-linked polyacrylamide, dextran, agarose or polysaccharide resins/gels), membranes (such as nitrocellulose or nylon membranes), beads (such as glass beads, agarose beads, cross-linked agarose beads, polystyrene beads, various coated and uncoated magnetic beads, polyacrylamide beads, latex beads and dimethylacrylamide beads), chitin, sand, pumice, glass, metal, silicon, rubber, polystyrene, polypropylene, polyvinylchloride, polyvinyl fluoride, polycarbonate, latex, diazotized paper, the internal surface of multi-well plates, and the like, wherein the solid support is insoluble under the conditions of the assay.

As indicated above, the solid support can be particulate (pellets, beads, and the like), or can be in the form of a continuous surface (membranes, meshes, plates, slides, disks, capillaries, hollow fibres, needles, pins, chips, solid fibres, gels, and the like). These supports can be modified as necessary with reactive groups that allow attachment of proteins or peptides, such as amino groups, carboxyl groups, sulphydryl groups, hydroxyl groups, activated versions of the preceding groups, and/or carbohydrate moieties. Examples of coupling chemistries that can be employed to immobilise the candidate PRE/TIM, target PKC or primary antibody on the solid support include cyanogen bromide activation, N-hydroxysuccinimide activation, epoxide activation, sulfhydryl activation, hydrazide activation, and carboxyl and amino derivatives for carbodiimide coupling chemistries.

Alternatively, the PRE/TIM, target PKC or primary antibody can be modified with a group that allows for attachment of the peptide or protein to an appropriately modified solid support. For example, a His-tag that allows the peptide/protein to be immobilised on a solid support modified to contain Ni²⁺ ions; biotin that allows the peptide/protein to be immobilised on a solid support modified to contain avidin/streptavidin, or an antigen that allows the peptide/protein to be immobilised on a solid support modified with the corresponding specific antibody. Other examples are known in the art and include the binding pairs described above.

Immobilisation of one or more component of the binding assay can facilitate “high-throughput” screening of candidate PREs/TIMs. High-throughput screening provides the advantage of processing a plurality samples simultaneously and significantly decreases the time required to screen a large number of samples. For high-throughput screening, reaction components are usually housed in a multi-container carrier or platform, such as a multi-well plate, which allows a plurality of assays each containing a different candidate PRE/TIM to be monitored simultaneously. Many high-throughput screening or assay systems are now available commercially, as are automation capabilities for many procedures such as sample and reagent pipetting, liquid dispensing, timed incubations, formatting samples into a high-throughput format and microplate readings in an appropriate detector, resulting in much faster throughput times.

### II. Protein Kinase Inhibition Assays

The TIMs of the present invention are capable of inhibiting the activity of one or more PKC isoforms, and optionally one or more other protein kinases. The ability of candidate TIMs to inhibit PKC activity, and the activity of other protein kinases, can initially be tested using standard in vitro assays. Assays to determine the activity of a variety of protein kinases are well known in the art, see for example, Current Protocols in Pharmacology (Enna & Williams, Ed., J. Wiley & Sons, New York, N.Y.).

In general, the ability of a candidate compound to inhibit the activity of a selected protein kinase is assessed by adding the candidate compound to a reaction mixture comprising the target protein kinase in an appropriate buffer, together with a substrate, ATP, and any necessary co-factors (such as phosphatidylserine, phorbol esters, Mn²⁺ and/or Ca²⁺). After a suitable incubation time, the extent of phosphorylation of the substrate is monitored and compared to a control reaction, for example, a reaction conducted in the absence of the candidate compound, or in the presence of a known PK inhibitor. The substrate used in the assay is a protein or a peptide that is capable of being phosphorylated by the particular protein kinase being investigated. In most assays, peptide substrates are used.

The extent of substrate phosphorylation can be determined by a number of methods known in the art, for example, traditional methods employ radiolabelled ATP in the assay and determine the amount of radioactivity incorporated into the phosphorylated substrate at the end of the incubation period.

Alternative methods known in the art include those that employ a suitably labelled monoclonal antibody, which specifically binds to the phosphorylated form of the substrate. The antibody is added to the reaction mixture during or at the end of the incubation period and the amount of bound antibody is measured as an indication of the amount of substrate phosphorylation that has taken place. Other methods include the use of fluorescently labelled substrates (see, for example, PepTag® Non-Radioactive Assays, Promega, Madison, Wis.), fluorescently labelled substrates together with a quencher molecule (for example, the IQ® Assays from Pierce Biotechnology Inc., Rockford, Ill.) and luminescent detection of unreacted ATP (for example, the Kinase-Glo™ Luminescent Kinase Assays from Promega, Madison, Wis.). Methods based on fluorescence polarisation techniques that include the addition, at the end of the incubation period, of a fluorescently labelled tracer molecule and an antibody capable of binding the phosphorylated substrate and the tracer molecule (see PanVera® PolarScreen™ kits from Invitrogen, Carlsbad, Calif.).

In vitro assays such as those outlined above can be performed as high-throughput assays, which allows a number of different candidate inhibitors to be screened simultaneously against a particular protein kinase. High-throughput assays also allow a particular TIM to be screened for activity against a panel of different protein kinases. Many commercially available protein kinase assay kits are specifically designed to permit high-throughput screening (for example, the IQ® assays, Kinase-Glo™ assays and PanVera® PolarScreen™ kits referred to above, and the Multiscreen®_{HTS}-PH Phosphocellulose Filter Plate Assays from Millipore, Billerica, Mass.).

The protein kinase employed in the in vitro assays can be in the form of a purified enzyme, a semi-purified enzyme, or it can be present in a partially purified or crude cell lysate prepared from a cell line or tissue of interest. A number of protein kinases are commercially available in pure or partially pure form (for example, from Sigma-Aldrich, St Louis, Mo.; Pierce Biotechnology Inc., Rockford, Ill.; and Promega Madison, Wis.).

The TIMs of the present invention can be assessed for their ability to inhibit one or more protein kinases in a cellular context by contacting a cell line of interest with the TIM and subsequently assessing protein kinase activity in a cell lysate prepared from the cells using standard methods, such as those described above. Alternatively, a selected cell line maintained under appropriate growth conditions can be treated with a candidate TIM and the extent of phosphorylation of a naturally-occurring substrate molecule present within the cells can be assessed and compared to untreated control cells, or cells treated with a known inhibitor of the target protein kinase. For example, a candidate TIM can be assessed for its ability to inhibit PKB activity by determining the amount of phospho-GSK-3 present in cells treated with the compound using commercially available antibodies against phospho-GSK3α (Cell Signaling Technology, Beverly, Mass.). Alternatively, the cells can be treated with a candidate TIM and an exogenous protein kinase substrate, such as myristoylated alanine-rich C-kinase substrate (MARCKS), and the extent of phosphorylation of the added substrate can be determined, for example, using commercially available antibodies against the phospho-substrate.

### III. Assays for In Vitro Physiological Activity

The TIMs of the present invention can further be assessed for their ability to modulate one or more PKC-mediated physiological effects in vitro. In the context of the present invention, PKC-mediated physiological effects include, but are not limited to, cell proliferation, cell migration/invasion, cell survival, apoptosis, gap junction formation, and drug-resistance (in particular, drug-resistance in cancer cells).

In general, the ability of a candidate TIM to inhibit a PKC-mediated physiological effect can be assessed by contacting cells in which the physiological effect is manifested with the candidate compound and incubating the cells under conditions suitable for assessing the physiological effect. If necessary, the cells can be treated with a reagent that promotes the uptake of the compound by the cells, for example, a reagent that promotes pinocytic endocytosis. The extent of modulation of the physiological effect can be determined by comparison of the test cells with a suitable control, for example, untreated cells incubated under the same conditions, or cells incubated under the same conditions in the presence of a known PKC inhibitor.

In accordance with one embodiment of the present invention, the TIMs inhibit cellular proliferation. Methods of assessing the ability of a candidate compound to inhibit cellular proliferation are well known in the art. In general, for in vitro assays, cells of a specific test cell line are grown to an appropriate density (e.g. approximately 1×10⁴) and the candidate compound is added. After an appropriate incubation time (for example, 48 to 74 hours), cell density is assessed. Methods of measuring cell density are known in the art, for example, the cell density can be assessed under a light inverted microscope by measuring the surface of the culture plate covered by the cell monolayer; or by using standard assays such as the resazurin reduction test (see Fields & Lancaster (1993) Am. Biotechnol. Lab. 11:48-50; O'Brien et al., (2000) Eur. J. Biochem. 267:5421-5426 and U.S. Pat. No. 5,501,959), the sulforhodamine assay (Rubinstein et al., (1990) J. Natl. Cancer Inst. 82:113-118), the neutral red dye test (Kitano et al., (1991) Euro. J. Clin. Investg. 21:53-58; West et al., (1992) J. Investigative Derm. 99:95-100), or the trypan blue exclusion assay. Alternatively, the cells can be detached from the plate, for example, by incubation with trypsin and then counted in an hemocytometer. Percent inhibition of proliferation of the cells can be calculated by comparison of the cell density in the treated culture with the cell density in control cultures, for example, cultures not pre-treated with the candidate compound and/or those pre-treated with a control compound known to inhibit cell proliferation. Cells may be treated with a mitogen prior to addition of the candidate compound to assess the ability of the compounds to inhibit proliferation of stimulated cells as opposed to unstimulated, or quiescent cells. The use of mitogen-stimulated cells can be useful, for example, in assessing the ability of the candidate compound to inhibit proliferation of endothelial cells.

DNA synthesis can be also assessed as an indication of cell proliferation. For example, by the uptake of [³H]thymidine. Typically cells are grown to an appropriate density (generally to confluence) at which point the growth medium is replaced with a medium that renders the cells quiescent (for example, DME 0.5% serum). The quiescent cells are exposed to a mitogenic stimulus, such as diluted serum or a growth factor, at a suitable interval after the medium replacement. [³H]thymidine is subsequently added to the cells, and the cells are maintained at 37° C. After an appropriate incubation time, the cells are washed, the acid-precipitable radioactivity is extracted and the amount of radioactivity determined, for example, by using a scintillation counter.

The above techniques can also be employed to assess cell survival and the effect of the TIMs on multi-drug resistant cells. Other techniques are known in the art (see, for example, Current Protocols in Pharmacology, Enna & Williams, Ed., J. Wiley & Sons, New York, N.Y.; Current Protocols in Cell Biology, Morgan, K., Ed., J. Wiley & Sons, New York, N.Y.).

A variety of readily available cell-lines can be utilised in the in vitro assays described above, including endothelial cells, cancer cells and keratinocytes. Non-limiting examples of suitable endothelial cell lines include human umbilical vein endothelial cells (HUVECs), bovine aortic endothelial cells (BAECs), human coronary artery endothelial cells (HCAECs), bovine adrenal gland capillary endothelial cells (BCE) and vascular smooth muscle cells. HUVECs can be isolated from umbilical cords using standard methods (see, for example, Jaffe et al. (1973) J. Clin. Invest. 52: 2745), or they can be obtained from the ATCC or various commercial sources, as can other suitable endothelial cell lines.

Exemplary cancer cell lines include, but are not limited to, ovarian cancer cell-lines OV90 and SK-OV-3, breast cancer cell-lines MCF-7 and MDA-MB-231, colon cancer cell-lines CaCo, HCT116 and HT29, cervical cancer cell-line HeLa, non-small cell lung carcinoma cell-lines A549, H661 and H1299, pancreatic cancer cell-lines MIA-PaCa-2 and AsPC-1, prostate cancer-cell line PC-3, bladder cancer cell-line T24, liver cancer cell-lineHepG2, brain cancer cell-line U-87 MG, melanoma cell-line A2058, lung cancer cell-line NCI-H460, and neuroblastoma cell line IMR-32. Other suitable cancer cell lines include those that are available from the American Type Culture Collection (ATCC), which currently provides 950 cancer cell lines.

Other examples of suitable cell lines include human keratinocytes (such as HaCaT cells); rheumatoid synovial fibroblasts (RSFs), and Jurkat T cells. Other suitable cell-lines are known in the art.

In general, the ability of a candidate TIM to inhibit cell migration can be assessed in vitro using standard cell migration assays and endothelial and/or cancer cells such as those described above. Typically, such assays are conducted in multi-well plates, the wells of the plate being separated by a suitable membrane into top and bottom sections. The membrane is coated with an appropriate compound, the selection of which is dependent on the type of cell being assessed and can be readily determined by one skilled in the art. Examples include collagen or gelatine for endothelial cells and Matrigel for neoplastic cell lines. An appropriate chemo-attractant, such as EGM-2, IL-8, aFGF, bFGF and the like, is added to the bottom chamber as a chemo-attractant. An aliquot of the test cells together with the candidate TIM are added to the upper chamber, typically various dilutions of the candidate TIM are tested. After a suitable incubation time, the membrane is rinsed, fixed and stained. The cells on the upper side of the membrane are wiped off, and then randomly selected fields on the bottom side are counted.

Various cell lines can be used in cell migration assays. Examples include the endothelial and cancer cells listed above.

Apoptosis and gap junction formation in cells treated with a candidate TIM can be assessed, for example, by standard immunocytochemical techniques. Non-limiting examples are provided in the Examples herein. Other techniques are known in the art (see, for example, Current Protocols in Pharmacology, Enna & Williams, Ed., J. Wiley & Sons, New York, N.Y.; Current Protocols in Cell Biology, Morgan, K., Ed., J. Wiley & Sons, New York, N.Y.).

### Assays for In Vivo Physiological Activity

The ability of the TIMs of the invention to inhibit one or more PKC-mediated physiological effects can be tested in vivo using an appropriate animal model known in the art (see, for example, Current Protocols in Pharmacology, Enna & Williams, Ed., J. Wiley & Sons, New York, N.Y.).

For example, the effect of a TIM on ischemia can be assessed ex vivo using Langendorff-perfused rat heart (see, for example, Yao, et al., (1994) Biol. Pharm. Bull. 17:517) or in vivo using rat or dog models of myocardial ischemia/reperfusion injury. The anti-atherosclerotic and anti-hypertensive effects can be assessed, for example, in spontaneously hypertensive rats (see, for example, Kubo, et al., (1992) J. Pharmacobtodyn. 15:657). A variety of animal models are known in the art to test the anti-inflammatory activity of test compounds, for example, carrageenan-induced paw edema, adjuvant-induced arthritis and carrageenan air pouch rat models (see Current Protocols in Pharmacology, Enna & Williams, Ed., J. Wiley & Sons, New York, N.Y.), and rat models of psoriasis (see, for example, Smith, S., et al. (1993) Immunopharmacol. Immunotoxicol. 15:13).

For assessing the ability of the TIMs to inhibit tumour growth or proliferation in vivo, standard animal models can be used, for example, xenograft models, in which a human tumour has been implanted into an animal. Examples of xenograft models of human cancer include, but are not limited to, human solid tumour xenografts, implanted by sub-cutaneous injection or implantation; human solid tumour isografts, implanted by fat pad injection and human solid tumour orthotopic xenografts, implanted directly into the relevant tissue, all of which can be used in tumour growth assays. Survival assays using experimental models of lymphoma and leukaemia in mice, and experimental models of lung metastasis in mice can also be employed.

For example, the TIMs can be tested in vivo on solid tumours using mice that are subcutaneously grafted bilaterally with 30 to 60 mg of a tumour fragment, or implanted with an appropriate number of cancer cells, on day 0. The animals bearing tumours are mixed before being subjected to the various treatments and controls. In the case of treatment of advanced tumours, tumours are allowed to develop to the desired size, animals having insufficiently developed tumours being eliminated. The selected animals are distributed at random to undergo the treatments and controls. Animals not bearing tumours may also be subjected to the same treatments as the tumour-bearing animals in order to be able to dissociate the toxic effect from the specific effect on the tumour. Chemotherapy generally begins from 3 to 22 days after grafting, depending on the type of tumour, and the animals are observed every day. The TIMs of the present invention can be administered to the animals, for example, by intraperitoneal (i.p.) injection or bolus infusion.

The tumours are measured after a pre-determined time period, or they can be monitored continuously by measuring about 2 or 3 times a week until the tumour reaches a pre-determined size and/or weight, or until the animal dies if this occurs before the tumour reaches the pre-determined size/weight. The animals are then sacrificed and the tissue histology, size and/or proliferation of the tumour assessed.

For the study of the effect of the TIMs on leukaemias, the animals are grafted with a particular number of cells, and the anti-tumour activity is determined by the increase in the survival time of the treated mice relative to the controls.

To study the effect of the TIMs on tumour metastasis, tumour cells are typically treated with the composition ex vivo and then injected into a suitable test animal. The spread of the tumour cells from the site of injection is then monitored over a suitable period of time.

Suitable cancer cell lines for in vivo testing of the compounds include those listed above.

In vivo toxic effects of the TIMs can be evaluated by measuring their effect on animal body weight during treatment and by performing haematological profiles and liver enzyme analysis after the animal has been sacrificed.

### Pharmaceutical Compositions

For administration to a subject, the present invention provides for pharmaceutical compositions comprising a TIM of the invention and one or more non-toxic pharmaceutically acceptable carriers, diluents, excipients and/or adjuvants. If desired, other therapeutic agents, including other TIMs, may be included in the compositions.

The pharmaceutical compositions may comprise from about 1% to about 95% of a TIM of the invention. Compositions formulated for administration in a single dose form may comprise, for example, about 20% to about 90% of the TIM, whereas compositions that are not in a single dose form may comprise, for example, from about 5% to about 20% of the TIM. Non-limiting examples of unit dose forms include dragées, tablets, ampoules, vials, suppositories and capsules.

The pharmaceutical compositions can be formulated for administration by a variety of routes. For example, the compositions can be formulated for oral, topical, rectal or parenteral administration or for administration by inhalation or spray. The term “parenteral” as used herein includes subcutaneous injections, intravenous, intramuscular, intrathecal, intrasternal injection or infusion techniques. For the treatment of cancer, intra-tumoral administration is also contemplated.

Pharmaceutical compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the TIM in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use can also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Pharmaceutical compositions formulated as aqueous suspensions contain the TIM in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Pharmaceutical compositions can be formulated as oily suspensions by suspending the TIM in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

The pharmaceutical compositions can be formulated as a dispersible powder or granules, which can subsequently be used to prepare an aqueous suspension by the addition of water. Such dispersible powders or granules provide the TIM in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Pharmaceutical compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Pharmaceutical compositions can be formulated as a syrup or elixir by combining the TIM with one or more sweetening agents, for example glycerol, propylene glycol, sorbitol or sucrose. Such formulations can also optionally contain one or more demulcents, preservatives, flavouring agents and/or colouring agents.

The pharmaceutical compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Other pharmaceutical compositions and methods of preparing pharmaceutical compositions are known in the art and are described, for example, in “Remington: The Science and Practice of Pharmacy” (formerly “Remington Pharmaceutical Sciences”); Gennaro, A., Lippincott, Williams & Wilkins, Philadelphia, Pa. (2000).

The TIM is included in the pharmaceutical compositions in an amount effective to achieve the intended purpose. Thus the term “therapeutically effective dose” refers to the amount of the TIM that ameliorates the symptoms of the PKC-α mediated disease or disorder to be treated. Determination of a therapeutically effective dose of a compound is well within the capability of those skilled in the art. For example, the therapeutically effective dose can be estimated initially either in cell culture assays, or in animal models, such as those described herein. Animal models can also be used to determine the appropriate concentration range and route of administration. Such information can then be used to determine useful doses and routes for administration in other animals, including humans using standard methods known in those of ordinary skill in the art.

Therapeutic efficacy and toxicity can also be determined by standard pharmaceutical procedures such as, for example, by determination of the median effective dose, or ED₅₀ (i.e. the dose therapeutically effective in 50% of the population) and the median lethal dose, or LD₅₀ (i.e. the dose lethal to 50% of the population). The dose ratio between therapeutic and toxic effects is known as the “therapeutic index,” which can be expressed as the ratio, LD₅₀/ED₅₀. The data obtained from cell culture assays and animal studies can be used to formulate a range of dosage for human or animal use. The dosage contained in such compositions is usually within a range of concentrations that include the ED₅₀ and demonstrate little or no toxicity. The dosage varies within this range depending upon the dosage form employed, sensitivity of the subject, and the route of administration and the like.

The exact dosage to be administered to a subject can be determined by the practitioner, in light of factors related to the subject requiring treatment. Dosage and administration are adjusted to provide sufficient levels of the TIM and/or to maintain the desired effect. Factors which may be taken into account when determining an appropriate dosage include the severity of the disease state, general health of the subject, age, weight, and gender of the subject, diet, time and frequency of administration, drug combination(s), reaction sensitivities, and tolerance/response to therapy. Dosing regimens can be designed by the practitioner depending on the above factors as well as factors such as the half-life and clearance rate of the particular formulation.

Exemplary daily doses for the TIMs of the invention range from about 0.0001 to about 100 mg per kilogram of body weight per day, for example, from about 0.001 to about 10 mg per kilogram, or from about 0.01 to about 5 mg per kilogram. The daily dose can be administered as a single dose or it can be divided into two, three, four, five, six or more sub-doses for separate administration at appropriate intervals throughout the day, optionally, in unit dosage forms.

### Method of Selecting Isoform-Specific PKC Inhibitors

The present invention provides for a method of selecting an isoform-specific TIM by first screening for a PRE that specifically binds to one isoform of PKC. The method generally comprises the steps of providing a library of candidate isoform-specific PREs, each PRE having a sequence represented by general formula (I), or the retro form thereof, screening the library against one or more PKC isoforms, and selecting a PRE having the desired isoform-specificity. This PRE can then be conjugated to a PKC inhibitor to provide the isoform-specific TIM

A “library” in this context comprises a plurality of candidate PREs, for example, between two and about 1000 candidate PREs. The size of the library can be selected based on the capacity of the screening technique being employed. For example, when high-throughput screening techniques are available, the library can comprise a large number of candidate PREs, such as between about 20 and about 1000 candidate PREs, or between about 50 and 1000 candidate PREs. When low throughput screening techniques are employed, the library can comprise a smaller number of candidate PREs, for example, between about two and about 50, or between about two and about 20 candidate PREs.

Libraries of candidate PREs can be readily prepared by standard peptide synthesis techniques, such as solid-phase peptide synthesis or solution peptide synthesis as described above. The candidate PREs can be screened for their affinity for a particular PKC isoform using assay methods such as those described above, for example, by a competitive or other binding assay. The candidate PREs can be screened against a single PKC isoform, or they can be screened against a plurality of different isoforms. The method can be readily adapted to high throughput, thus allowing large numbers of candidate PREs to be screened and/or allowing candidate PREs to be screened against a plurality of PKCs simultaneously.

### Uses of the Targeted Inhibitory Molecules

The TIMs of the present invention have numerous applications in the areas of therapeutics, as well as in research settings and development of PKC antagonists and agonists.

The present invention provides for the use of the TIMs to inhibit the activity of one or more PKC isoforms, and optionally one or more other protein kinases in vitro or in vivo and for methods of inhibiting one or more PKC isoforms and optionally one or more other protein kinases in a subject by administration of an effective amount of a TIM of the invention.

PKCs have been implicated in a variety of diseases and disorders. Accordingly, the present invention contemplates the use of the TIMs, alone or in combination with other therapeutic agents, in the treatment of PKC-related diseases and disorders such as, cancer, psoriasis, angiogenesis, restenosis, atherosclerosis, cardiovascular disease (such as arrhythmia), hypertension, diabetes, neurological disorders, rheumatoid arthritis, kidney disorders (such as polycystic kidney), inflammatory disorders and autoimmune disorders.

One embodiment of the present invention provides for the use of the TIMs in the treatment of a PKC-α related disorder, such as cancer, complications of diabetes (including retinopathy, high blood pressure, diabetes-dependent cardiovascular disease), polycystic kidney disease, hypertension, heart hypertrophy and heart failure.

Another embodiment of the present invention provides for the use of the TIMs in the treatment of cancer. In this context, treatment with a TIM of the invention may result in a reduction in the size of a tumour, the slowing or prevention of an increase in the size of a tumour, an increase in the disease-free survival time between the disappearance or removal of a tumour and its reappearance, prevention of an initial or subsequent occurrence of a tumour (e.g. metastasis), an increase in the time to progression, reduction of one or more adverse symptom associated with a tumour, or an increase in the overall survival time of a subject having cancer.

The TIMs can be used to inhibit the growth and/or metastasis of a variety of tumours. Exemplary tumours include, but are not limited to, haematologic neoplasms, including leukaemias, myelomas and lymphomas; carcinomas, including adenocarcinomas and squamous cell carcinomas; melanomas and sarcomas. Carcinomas, melanomas and sarcomas are also frequently referred to as “solid tumours” or “solid cancers.” Examples of commonly occurring solid tumours and cancers include, but are not limited to, cancer of the brain, breast, cervix, colon, head and neck, kidney, lung (including non-small cell and small cell), ovary, pancreas, prostate, stomach, rectum and uterus. Various forms of lymphoma also may result in the formation of a solid tumour and, therefore, are also often considered to be solid tumours.

Additional cancers encompassed by the present invention include, for example, multiple myeloma, neuroblastoma, rhabdomyosarcoma, primary thrombocytosis, primary macroglobulinemia, primary brain tumours, gliomas, mesothelioma and medulloblastoma.

In one embodiment of the present invention, the TIMs are used in the treatment of a solid cancer. In another embodiment of the invention, the TIMs are used in the treatment of brain cancer, breast cancer, colon cancer, lung cancer, malignant melanoma, ovarian cancer, prostate cancer, neuroblastoma, glioma, colorectal cancer or thyroid cancer. In a further embodiment, the TIMs are used in the treatment of a cancer in which upregulation of PKC-α expression is known to occur, for example, urinary bladder cancer, prostate cancer and endometrial cancer.

The TIMs can also be used to treat drug resistant cancers, including multidrug resistant tumours. As is known in the art, the resistance of cancer cells to chemotherapy is one of the central problems in the management of cancer. In one embodiment of the present invention, the TIMs are used to decrease or reverse the drug-resistance of a cancer cell. In one embodiment, the TIMs are used in the treatment of a drug-resistant cancer in which upregulation of PKC-α expression is known to occur, for example, drug-resistant colon, colorectal or breast cancer.

Certain cancers, such as prostate and breast cancer, can be treated by hormone therapy, i.e. with hormones or anti-hormone drugs that slow or stop the growth of certain cancers by blocking the body's natural hormones. Such cancers may develop resistance, or be intrinsically resistant, to hormone therapy. The present invention further contemplates the use of the PKI compounds in the treatment of such “hormone-resistant” or “hormone-refractory” cancers.

The present invention also contemplates the use of the TIMs as “sensitizing agents.” In this case, the TIM alone does not have a cytotoxic effect on the cancer cells, but provides a means of weakening the cells or decreasing their resistance to one or more standard chemotherapeutics, and thereby facilitates the benefit from conventional anti-cancer therapeutics.

The cancer to be treated may be indolent or it may be aggressive. The present invention contemplates the use of the TIMs in the treatment of refractory cancers, advanced cancers, recurrent cancers and metastatic cancers.

“Aggressive cancer,” as used herein, refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of another cancer type. A “refractory” cancer or tumour refers to a cancer or tumour that has not responded to treatment. “Advanced cancer,” refers to overt disease in a patient, wherein such overt disease is not amenable to cure by local modalities of treatment, such as surgery or radiotherapy. Advanced disease may refer to a locally advanced cancer or it may refer to metastatic cancer. The term “metastatic cancer” refers to cancer that has spread from one part of the body to another. Advanced cancers may also be unresectable, that is, they have spread to surrounding tissue and cannot be surgically removed.

The present invention contemplates the use of the TIMs at various stages in tumour development and progression. Thus, the present invention contemplates the use of the TIMs as part of a primary therapy, a neo-adjuvant therapy (to primary therapy), or as part of an adjuvant therapy regimen, where the intention is to cure the cancer in a subject.

As is known in the art with respect to the treatment of a cancer, “primary therapy” refers to a first line of treatment upon the initial diagnosis of cancer in a subject. Exemplary primary therapies may involve surgery, a wide range of chemotherapies and radiotherapy. “Adjuvant therapy” refers to a therapy that follows a primary therapy and that is administered to subjects at risk of relapsing. Adjuvant systemic therapy is begun soon after primary therapy to delay recurrence, prolong survival or cure a subject.

The TIMs can be used alone or in combination with one or more other chemotherapeutic agents. Combinations of the TIMs and standard chemotherapeutics may act to improve the efficacy of the chemotherapeutic and, therefore, can be used to improve standard cancer therapies. This application is particularly important in the treatment of drug-resistant cancers which are not responsive to standard treatment. In one embodiment, the TIMs of the invention are used in combination therapy with one or more standard chemotherapeutics. In another embodiment, the TIMs of the invention are used in combination with one or more standard chemotherapeutics for the treatment of drug-resistant cancer.

### Clinical Trials

One skilled in the art will appreciate that, following the demonstrated effectiveness of a TIM of the invention in vitro and in animal models, the TIM will enter clinical trials in order to further evaluate its efficacy and to obtain regulatory approval for therapeutic use. The details of any given clinical trial will vary depending upon the disease being evaluated, but follow a general format which is exemplified below with respect to the clinical trial protocol for the evaluation of a therapeutic for the treatment of cancer.

As is known in the art, clinical trials progress through phases of testing, which are identified as Phases I, II, III, and IV.

Initially a TIM will be evaluated in a Phase I trial. Typically Phase I trials are used to determine the best mode of administration (for example, by pill or by injection), the frequency of administration, and the toxicity for the compounds. Phase I studies frequently include laboratory tests, such as blood tests and biopsies, to evaluate the effects of a compound in the body of the patient. For a Phase I trial, a small group of cancer patients are treated with a specific dose of the TIM. During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to further evaluate the effectiveness and safety of the TIM. In Phase U trials, the TIM is administered to groups of patients with either one specific type of cancer or with related cancers, using the dosage found to be effective in Phase I trials.

Phase III trials focus on determining how a compound compares to the standard, or most widely accepted, treatment. In Phase III trials, patients are randomly assigned to one of two or more “arms”. In a trial with two arms, for example, one arm will receive the standard treatment (control group) and the other arm will receive treatment with the TIM (investigational group).

Phase N trials are used to further evaluate the long-term safety and effectiveness of a compound. Phase IV trials are less common than Phase I, II and III trials and will take place after the TIM has been approved for standard use.

### Eligibility of Patients for Clinical Trials

Participant eligibility criteria can range from general (for example, age, sex, type of cancer) to specific (for example, type and number of prior treatments, tumour characteristics, blood cell counts, organ function). Eligibility criteria may also vary with trial phase. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I cancer trials usually comprise 15 to 30 participants for whom other treatment options have not been effective. Phase II trials typically comprise up to 100 participants who have already received chemotherapy, surgery, or radiation treatment, but for whom the treatment has not been effective. Participation in Phase II trials is often restricted based on the previous treatment received. Phase III trials usually comprise hundreds to thousands of participants. This large number of participants is necessary in order to determine whether there are true differences between the effectiveness of the TIM and the standard treatment. Phase III may comprise patients ranging from those newly diagnosed with cancer to those with extensive disease in order to cover the disease continuum.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

### Assessment of Patients Prior to Treatment

Prior to commencement of the study, several measures known in the art can be used to first classify the patients. Patients can first be assessed, for example, using the Eastern Cooperative Oncology Group (ECOG) Performance Status (PS) scale. ECOG PS is a widely accepted standard for the assessment of the progression of a patient's disease as measured by functional impairment in the patient, with ECOG PS 0 indicating no functional impairment, ECOG PS 1 and 2 indicating that the patients have progressively greater functional impairment but are still ambulatory and ECOG PS 3 and 4 indicating progressive disablement and lack of mobility.

Patients' overall quality of life can be assessed, for example, using the McGill Quality of Life Questionnaire (MQOL) (Cohen et al (1995) Palliative Medicine 9: 207-219). The MQOL measures physical symptoms; physical, psychological and existential well-being; support; and overall quality of life. To assess symptoms such as nausea, mood, appetite, insomnia, mobility and fatigue the Symptom Distress Scale (SDS) developed by McCorkle and Young ((1978) Cancer Nursing 1: 373-378) can be used.

Patients can also be classified according to the type and/or stage of their disease and/or by tumour size.

### Pharmacokinetic Monitoring

To fulfil Phase I criteria, distribution of the TIM is monitored, for example, by chemical analysis of samples, such as blood or urine, collected at regular intervals. For example, samples can be taken at regular intervals up until about 72 hours after the start of infusion.

If analysis is not conducted immediately, the samples can be placed on dry ice after collection and subsequently transported to a freezer to be stored at −70° C. until analysis can be conducted. Samples can be prepared for analysis using standard techniques known in the art and the amount of the TIM present can be determined, for example, by high-performance liquid chromatography (HPLC).

Pharmacokinetic data can be generated and analyzed in collaboration with an expert clinical pharmacologist and used to determine, for example, clearance, half-life and maximum plasma concentration.

### Monitoring of Patient Outcome

The endpoint of a clinical trial is a measurable outcome that indicates the effectiveness of a compound under evaluation. The endpoint is established prior to the commencement of the trial and will vary depending on the type and phase of the clinical trial. Examples of endpoints include, for example, tumour response rate—the proportion of trial participants whose tumour was reduced in size by a specific amount, usually described as a percentage; disease-free survival—the amount of time a participant survives without cancer occurring or recurring, usually measured in months; overall survival—the amount of time a participant lives, typically measured from the beginning of the clinical trial until the time of death. For advanced and/or metastatic cancers, disease stabilization—the proportion of trial participants whose disease has stabilized, for example, whose tumour(s) has ceased to grow and/or metastasize, can be used as an endpoint. Other endpoints include toxicity and quality of life.

Tumour response rate is a typical endpoint in Phase II trials. However, even if a treatment reduces the size of a participant's tumour and lengthens the period of disease-free survival, it may not lengthen overall survival. In such a case, side effects and failure to extend overall survival might outweigh the benefit of longer disease-free survival. Alternatively, the participant's improved quality of life during the tumour-free interval might outweigh other factors. Thus, because tumour response rates are often temporary and may not translate into long-term survival benefits for the participant, response rate is a reasonable measure of a treatment's effectiveness in a Phase II trial, whereas participant survival and quality of life are typically used as endpoints in a Phase III trial.

### Kits

### Research Kits

The present invention provides for kits comprising one or more TIM for research applications. The TIM(s) provided in the kit can incorporate a detectable label, such as a fluorophore, radioactive moiety, enzyme, biotin/avidin label, chromophore, chemiluminescent label, or the like, or the kit may include reagents for labelling the TIM. The TIM can be provided in a single container, aliquoted into separate containers, or pre-dispensed into an appropriate assay format, for example, into microtitre plates and/or immobilised on a solid support.

The kits can optionally include reagents useful for conducting screening assays, such as buffers, salts, antibodies, enzymes, enzyme co-factors, substrates, culture media, detection reagents, and the like. Other components, such as buffers and solutions for the isolation and/or treatment of a test sample, may also be included in the kit. The kit may additionally include one or more controls, such as a purified or partially purified PKC.

One or more of the components of the kit may be lyophilised and the kit may further comprise reagents suitable for the reconstitution of the lyophilised components. The various components of the kit are provided in suitable containers. For example, for screening and diagnostic purposes one or more of the containers may be a microtitre plate. Where appropriate, the kit may also optionally contain reaction vessels, mixing vessels and other components that facilitate the preparation of reagents or the test sample. The kit may also include one or more instrument for assisting with obtaining a test sample, such as a syringe, pipette, forceps, measured spoon, or the like.

The kit can optionally include instructions for use, which may be provided in paper form or in computer-readable form, such as a disc, CD, DVD or the like.

### Pharmaceutical Kits

The present invention additionally provides for therapeutic kits or packs containing one or more of the TIMs of the invention or one or more pharmaceutical compositions comprising the TIMs. The kits and packs can be used in the treatment of protein kinase mediated diseases or disorders. Individual components of the kit can be packaged in separate containers, associated with which, when applicable, can be a notice in the form prescribed by a governmental agency regulating the manufacture, use or sale of pharmaceuticals or biological products, which notice reflects approval by the agency of manufacture, use or sale for human or animal administration. The kit can optionally further contain one or more other therapeutic agents for use in combination with the TIMs of the invention. The kit may optionally contain instructions or directions outlining the method of use or dosing regimen for the TIMs and/or additional therapeutic agents.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

The invention will now be described with reference to specific examples. It will be understood that the following examples are intended to describe embodiments of the invention and are not intended to limit the invention in any way.

### EXAMPLES

The following peptide recognition elements were made by standard solid phase synthetic procedures.

TABLE 4
Sequences of Exemplary PREs

| Peptide | Sequence | SEQ ID NO |
|---|---|---|
| PRE 1 | RRKKGGKDFVVKR | 1 |
| PRE 2 | KDAQNLIGISL-NH₂ | 11 |
| PRE 3 | Ac-AKGIQEVKGGDAQNLIGISI-NH₂ | 12 |
| PRE 4 | Ac-KDAQNLIGISI-NH₂ | 13 |
| PRE 5 | Ac-AKGIQEVKGGKDAQNLIGISI-NH₂ | 14 |
| PRE 6 | Ac-KDANQLIGISI-NH₂ | 16 |
| PRE 7 | Ac-ISIGILQNADK-NH₂ | 17 |
| PRE 8 | ILEDKGGDAQNLIGISI | 5 |
| PRE 9 | Ac-isigilqnadk-NH₂ | 18 |
| PRE 10 | Ac-ISIGILNQADK-NH₂ | 19 |
| PRE 11 | Ac-RDAQNLIGISI-NH₂ | 20 |
| PRE 12 | Ac-KDAQNLI-NH₂ | 21 |
| PRE 13 | Ac-RDAQNLI-NH₂ | 22 |

As demonstrated in Examples 1-10 below, all the PREs tested have an affinity for at least one PKC isoform, and some are specific for one isoform or a group of isoforms. As it was expected that the measured level of specificity of the binding of the PREs to the various PKC isoforms may vary somewhat depending on the protocol selected for testing, several procedures were used to assess the binding specificity of the PRE as described below. Possible causes of variation between and within protocols include the fact that the PKC isoform specific primary antibodies do not bind their target to the same degree, which does not allow for quantitative comparison among isoforms, but does allow for a precise comparison of dose response of PRE-binding to a particular isoform. In addition, when using commercially purified enzymes, the preparations may include partially unfolded protein, which can alter the binding capacity assessment for the PRE binding, and when using cell extracts, which contain a complex mixture of molecules, unknown molecules may compete for PRE binding. Finally, in cells, an excess of PRE may saturate the binding site of its targeted isoform depending of the intracellular content of this isoform and its sublocalization. Despite these limitations of the different procedures, one skilled in the art will appreciate that the results provide a good indication of the overall binding and specificity of each of the tested PREs.

### Example 1

### In Vitro Competition Experiments with PREs and PKC-α: Protocol A

The ability of the PRE 1, 2 and 3 (see Table 3 above) to interfere with the binding of a PKC-α-specific polyclonal antibody to PKC-α was determined using the following protocol.

Cell lysates from either IMR-32 (human neuroblastoma) cells or C6Cx43 cells (rat glioma transfected cells overexpressing connexin 43) were obtained using standard protocols and the proteins of the lysate were separated by SDS PAGE electrophoresis and electrotransferred onto a nitrocellulose membrane. The membrane was incubated for 30 minutes in blocking buffer (TBST) containing the test peptide at either 5× or 20× the concentrations of the primary antibody. A primary polyclonal antibody specific for PKC-α (Santa Cruz Biotechnology, Inc., CA) was then added (15 μg/ml) and the membrane incubated for a further 45 minutes. Finally, the primary antibody was detected with a secondary antibody conjugated to alkaline phosphatase using standard procedures. The intensity of the band corresponding to PKC-α was assessed by scanning and densitometry using the Gel-Pro software (Media Cybernetics) to obtain relative band intensities (average of 3 replicas). Control assays were conducted as described above except that blocking buffer without peptide was used.

The results are summarised in Tables 5 and 6 below. The results are expressed as relative band intensity and as a percentage of the intensity of the corresponding band in the control assay (“Relative intensity (%)”). “% inhibition” relates to the percentage of the PKC-α band that is inaccessible to the antibody.

The results clearly indicate that both PRE 1 and PRE 2 mask the antigenic site of PKC-α on the membrane and that PRE 2 appears to be more efficient in this regard than PRE 1. Under these assay conditions, PRE 3 did not show an effect on antibody binding.

TABLE 5
Inhibition of Antibody Binding to PKC-α by PRE
1 and PRE 2 in IMR-32 Neuroblastoma Cells: Protocol A

| | | | Relative Intensity | Inhibition |
|---|---|---|---|---|
| Peptide | | Band Intensity | (%) | (%) |
| None (control) | | 2.087 | — | — |
| PRE 1 | (75 μg) | 1.040 | 49.8 | 50.2 |
| | (300 μg) | 0.771 | 36.9 | 63.1 |
| PRE 2 | (75 μg) | 0.917 | 43.9 | 56.1 |
| | (300 μg) | 0.192 | 9.1 | 90.9 |

TABLE 6
Inhibition of Antibody Binding to PKC-α by PRE 3: Protocol A

| | IMR-32 Cells | | | C6Cx43 Cells | | |
|---|---|---|---|---|---|---|
| | | Relative | | | Relative | |
| | Band | Intensity | Inhibition | Band | Intensity | Inhibition |
| Peptide | Intensity | (%) | (%) | Intensity | (%) | (%) |
| None | 0.895 | — | — | 0.927 | — | — |
| (control) | | | | | | |
| PRE 3 | | | | | | |
| (75 μg) | 0.889 | 99.3 | 0.7 | 0.909 | 98.0 | 0.0 |
| (300 μg) | 0.922 | 103.0 | 0.0 | 0.888 | 95.8 | 0.0 |
| (600 μg) | 0.888 | 99.2 | 0.0 | 0.889 | 95.9 | 0.0 |
| None | 0.591 | — | — | 0.572 | — | — |
| (control) | | | | | | |
| PRE 3 | | | | | | |
| (3.75 mg) | 0.600 | 101.5 | 0.0 | 0.603 | 105.4 | 0.0 |
| (5.25 mg) | 0.603 | 102.0 | 0.0 | 0.599 | 104.7 | 0.0 |
| (7.5 mg) | 0.609 | 103.0 | 0.0 | 0.589 | 103.0 | 0.0 |

### Example 2

### In Vitro Competition Experiments with PREs and PKC-β: Protocol A

Peptides PRE 1, PRE 2 and PRE 3 (see Table 4) were tested for their ability to interfere with the binding of a PKC-β-specific polyclonal antibody (Santa Cruz Biotechnology, Inc.) to PKC-β using the general protocol described in Example 1. The results are shown in Table 7 and show that there is some cross reactivity between both PRE 1 and PRE 2 and PKC-β. It is worth noting in this regard that PKC-α and PKC-β belong to the same sub-group of PKCs (cPKCs). The effect with PKC-β, however, is fairly limited indicating that these two peptides have a reasonable degree of specificity for PKC-α. Under these assay conditions, PRE 3 did not show an effect on antibody binding to PKC-β.

TABLE 7
Inhibition of Antibody Binding to PKC-β by PRE 1,
PRE 2 and PRE 3 in IMR-32 Neuroblastoma Cells: Protocol A

| | | | Relative Intensity | Inhibition |
|---|---|---|---|---|
| Peptide | | Band Intensity | (%) | (%) |
| None (control) | | 1.160 | — | — |
| PRE 1 | (75 μg) | 0.825 | 71.1 | 28.9 |
| | (300 μg) | 0.528 | 45.5 | 54.5 |
| PRE 2 | (75 μg) | 0.800 | 69.0 | 31.0 |
| | (300 μg) | 0.403 | 34.7 | 65.3 |
| None (control) | | 1.855 | — | — |
| PRE 3 | (75 μg) | 1.900 | 102.4 | 0.0 |
| | (300 μg) | 1.900 | 102.4 | 0.0 |
| | (600 μg) | 1.847 | 99.6 | 0.0 |

### Example 3

### In Vitro Competition Experiments with PREs and PKC-α: Protocol B

The ability of the peptides PRE 2 and PRE 3 (see Table 4) to interfere with the binding of a PKC-α-specific polyclonal antibody (Santa Cruz Biotechnology, Inc.) to PKC-α was determined using a modified version of the protocol outlined above in which the test peptide was added directly to the cell extract prior to electrophoresis at a concentration of either 5× or 15× the concentration of the protein applied to each well of the gel for the Western blots (20 μg).

The results are shown in Table 8. The results show that the interaction between each peptide and PKC-α was sufficiently strong to prevent dissociation during electrophoresis and that both PRE 2 and PRE 3 effectively interfered with PKC-α antibody binding to PKC-α. PRE 3 was more efficient than PRE 2 under these assay conditions.

TABLE 8
Inhibition of Antibody Binding to PKC-α by PRE 2 and
PRE 3 in IMR-32 Neuroblastoma Cells: Protocol B

| | | | Relative Intensity | |
|---|---|---|---|---|
| Peptide | | Band Intensity | (%) | Inhibition (%) |
| None (control) | | 0.759 | — | — |
| PRE 2 | (100 μg) | 0.0103 | 0.135 | 99.9 |
| | (200 μg) | 0.0 | 0.0 | 100.0 |
| | (300 μg) | 0.0 | 0.0 | 100.0 |
| PRE 3 | (100 μg) | 0.0 | 0.0 | 100.0 |
| | (200 μg) | 0.0 | 0.0 | 100.0 |
| | (300 μg) | 0.0 | 0.0 | 100.0 |

### Example 4

### In Vitro Competition Experiments with PREs and PKC-β: Protocol B

Peptides PRE 2 and PRE 3 (see Table 4) were tested for their ability to interfere with the binding of a PKC-β-specific polyclonal antibody (Santa Cruz Biotechnology, Inc.) to PKC-β using the general protocol described in Example 3. The results are shown in Table 9 and show that there is some cross reactivity between PRE 2 and PKC-β. At low concentrations, however, the effect is fairly limited indicating that PRE 2 has a reasonable degree of specificity for PKC-α when used at lower concentrations under these assay conditions. PRE 3 showed a similar effect on antibody binding to PKC-β under these conditions to that shown on antibody binding to PKC-α.

TABLE 9
Inhibition of Antibody Binding to PKC-β by PRE 2 and
PRE 3 in IMR-32 Neuroblastoma Cells: Protocol B

| | | | Relative Intensity | |
|---|---|---|---|---|
| Peptide | | Band Intensity | (%) | Inhibition (%) |
| None (control) | | 0.071 | — | — |
| PRE 2 | (100 μg) | 0.0322 | 45.3 | 54.7 |
| | (200 μg) | 0.0 | 0.0 | 100.0 |
| | (300 μg) | 0.0 | 0.0 | 100.0 |
| PRE 3 | (100 μg) | 0.0 | 0.0 | 100.0 |
| | (200 μg) | 0.0 | 0.0 | 100.0 |
| | (300 μg) | 0.0 | 0.0 | 100.0 |

### Example 5

### In Vitro Competition Experiments with PREs and PKC-α, PKC-βI and PKC-βII: Protocol B

Peptides PRE 3 and PRE 4 (see Table 4) were tested for their ability to interfere with the binding of isoform-specific polyclonal antibodies (Santa Cruz Biotechnology, Inc.) to PKC-α, PKC-βI or PKC-βII using the general protocol described in Example 3. The results are shown in Table 10. The results indicate that while PRE 4 shows some cross-reactivity with PKC-βI and PKC-βII, at low concentrations this peptide is reasonably specific for PKC-α. In agreement with the results shown in Table 9 above, PRE 3 showed a similar effect on antibody binding to PKC-βI and PKC-βII under these conditions to that shown on antibody binding to PKC-α.

TABLE 10
Inhibition of Antibody Binding to PKC-α, PKC-βI and
PKC-βII by PRE 3 and PRE 4 in IMR-32 Neuroblastoma
Cells: Protocol B

| | | |
|---|---|---|
| 100 μg | 200 μg | 300 μg |

PKC-α Control Band intensity: 302.5

| | | | |
|---|---|---|---|
| +PRE 3 | | | |
| Band Intensity | 0.0 | 0.0 | 0.0 |
| Relative Intensity (%) | 0.0 | 0.0 | 0.0 |
| Inhibition (%) | 100.0 | 100.0 | 100.0 |
| +PRE 4 | | | |
| Band Intensity | 32.2 | 1.5 | 0.0 |
| Relative Intensity (%) | 10.6 | 0.5 | 0.0 |
| Inhibition (%) | 89.4 | 99.5 | 100.0 |

PKC-βI Control Band intensity: 170.5

| | | | |
|---|---|---|---|
| +PRE 3 | | | |
| Band Intensity | 0.0 | 0.0 | 0.0 |
| Relative Intensity (%) | 0.0 | 0.0 | 0.0 |
| Inhibition (%) | 100.0 | 100.0 | 100.0 |
| +PRE 4 | | | |
| Band Intensity | 112.5 | 0.0 | 0.0 |
| Relative Intensity (%) | 66.0 | 0.0 | 0.0 |
| Inhibition (%) | 34.0 | 100.0 | 100.0 |

PKC-βII Control Band intensity: 98.6

| | | | |
|---|---|---|---|
| +PRE 3 | | | |
| Band Intensity | 0.0 | 0.0 | 0.0 |
| Relative Intensity (%) | 0.0 | 0.0 | 0.0 |
| Inhibition (%) | 100.0 | 100.0 | 100.0 |
| +PRE 4 | | | |
| Band Intensity | 69.58 | 0.0 | 0.0 |
| Relative Intensity (%) | 69.5 | 0.0 | 0.0 |
| Inhibition (%) | 29.0 | 100.0 | 100.0 |

### Example 6

### In Vitro Competition Experiments with PREs and PKC-ε: Protocol B

Peptides PRE 2, PRE 3 and PRE 4 (see Table 4) were tested for their ability to interfere with the binding of isoform-specific polyclonal antibodies (Santa Cruz Biotechnology, Inc.) to PKC-α using the general protocol described in Example 3. Two bands, representing alternate splicing variants of PKC-ε, were identified on the Western blot using this anti-PKC-ε antibody. The results with respect to both bands are summarised in Table 11. The results indicate that while PRE 2 and PRE 4 show some cross-reactivity with PKC-ε at low concentrations, these peptides are reasonably specific for PKC-α. PRE 3 showed a similar effect on antibody binding to PKC-ε under these conditions to that shown on antibody binding to PKC-α.

TABLE 11
Inhibition of Antibody Binding to PKC-ε by PRE 3 and PRE 4 in IMR-
32 Neuroblastoma Cells: Protocol B

| | PKC-ε Band 1 | | | PKC-ε Band 2 | | |
|---|---|---|---|---|---|---|
| | | Relative | | | Relative | |
| | Band | Intensity | Inhibition | Band | Intensity | Inhibition |
| Peptide | Intensity | (%) | (%) | Intensity | (%) | (%) |
| None (control) | 420.92 | — | — | 260.97 | — | — |
| PRE 2 | | | | | | |
| (20 μg) | 322.51 | 76.62 | 23.4 | 160.35 | 61.4 | 38.6 |
| (50 μg) | 0.0 | 0.0 | 100.0 | 0.0 | 0.0 | 100.0 |
| (100 μg) | 0.0 | 0.0 | 100.0 | 0.0 | 0.0 | 100.0 |
| None (control) | 420.16 | — | — | 280.95 | — | — |
| PRE 3 | | | | | | |
| (20 μg) | 5.12 | 0.12 | 99.0 | 0.0 | 0.0 | 100.0 |
| (50 μg) | 0.0 | 0.0 | 100.0 | 0.0 | 0.0 | 100.0 |
| (100 μg) | 0.0 | 0.0 | 100.0 | 0.0 | 0.0 | 100.0 |
| None (control) | 323.11 | — | — | 286.22 | — | — |
| PRE 4 | | | | | | |
| (20 μg) | 184.38 | 57.1 | 42.9 | 152.66 | 53.33 | 46.6 |
| (50 μg) | 0.0 | 0.0 | 100.0 | 0.0 | 0.0 | 100.0 |
| (100 μg) | 0.0 | 0.0 | 100.0 | 0.0 | 0.0 | 100.0 |

### Example 7

### In Vitro Toxicity Tests with PREs

In vitro cytotoxicity testing of the peptides PRE 3, PRE 4 and PRE 5 (see Table 4) was conducted following the general protocol outlined below (modified from “Fluorimetric DNA assay for cell growth estimation” Rao J, Otto W., Analytical Biochem. 207:186-192, 1992).

The assay was performed in 96 well plates, with 3,000 IMR-32 neuroblastoma cells seeded per well and 8 replicas were performed per treatment. The cells were pre-treated with either plain medium and a pinocytic endocytosis reagent (Molecular Probes) or medium containing the PRE under evaluation and the pinocytic endocytosis reagent. The cells were allowed to grow under conventional conditions for 3 days. The DNA content of each well was assessed at 24, 48 and 72 hours using Hoechst reagent according to standard procedures. The fluorescence intensity per well was measured using the plate reader “CytoFluor 2350” from Millipore. Excitation was 360 nm and emission was 460 nm. The number of cells is directly correlated to the DNA content.

The results are shown in Tables 12-14 as the average relative fluorescence intensity measured for the 8 replica wells, as well as the percentage of survival as compared with matching untreated controls. No cytotoxicity was observed for any of the tested peptides.

TABLE 12
IMR-32 Cell Survival after Treatment with PRE 4

| | 24 h | | 48 h | | 72 h | |
|---|---|---|---|---|---|---|
| | AFI* | % survival | AFI* | % survival | AFI* | % survival |
| Untreated | 124 | 100 | 266 | 100 | 915 | 100 |
| Control Cells | | | | | | |
| Cells + | 169 | 100 | 244 | 100 | 706 | 100 |
| Pinocytic | | | | | | |
| Endocytosis | | | | | | |
| Reagent | | | | | | |
| PRE 4 | | | | | | |
| 2.5 mg/ml | 179 | 106 | 240 | 99 | 777 | 110 |
| 10 mg/ml | 173 | 103 | 247 | 101 | 809 | 115 |

*AFI = Average Fluorescence Intensity

TABLE 13
IMR-32 Cell Survival after Treatment with PRE 3

| | 24 h | | 48 h | | 72 h | |
|---|---|---|---|---|---|---|
| | AFI* | % survival | AFI* | % survival | AFI* | % survival |
| Untreated | 152 | 100 | 362 | 100 | 820 | 100 |
| Control Cells | | | | | | |
| Cells + | 149 | 100 | 260 | 100 | 709 | 100 |
| Pinocytic | | | | | | |
| Endocytosis | | | | | | |
| Reagent | | | | | | |
| PRE 3 | | | | | | |
| 2.5 mg/ml | 193 | 127 | 389 | 150 | 871 | 123 |
| 10 mg/ml | 182 | 120 | 334 | 129 | 830 | 117 |

*AFI = Average Fluorescence Intensity

TABLE 14
IMR-32 Cell Survival after Treatment with PRE 5

| | 24 h | | 48 h | | 72 h | |
|---|---|---|---|---|---|---|
| | AFI* | % survival | AFI* | % survival | AFI* | % survival |
| Untreated | 633 | 100 | 929 | 100 | 1286 | 100 |
| Control Cells | | | | | | |
| PRE 5 | | | | | | |
| 62.5 μg/ml | 717 | 113 | 1084 | 117 | 1290 | 100 |
| 125 μg/ml | 593 | 94 | 991 | 107 | 1200 | 93 |
| 250 μg/ml | 557 | 88 | 899 | 97 | 1150 | 89 |
| 500 μg/ml | 492 | 78 | 891 | 96 | 1082 | 84 |

*AFI = Average Fluorescence Intensity

### Example 8

### Effect of PRE 3 and PRE 4 on the Subcellular Localisation of PKC-α

Peptides PRE 3 or PRE 4 (10 mg/ml) were introduced into human neuroblastoma cells (IMR-32) by pinocytic endocytosis. The cells were fixed and stained with rabbit PKC-α primary antibody and anti-rabbit Alexa-488 or Alexa-800 secondary antibody.

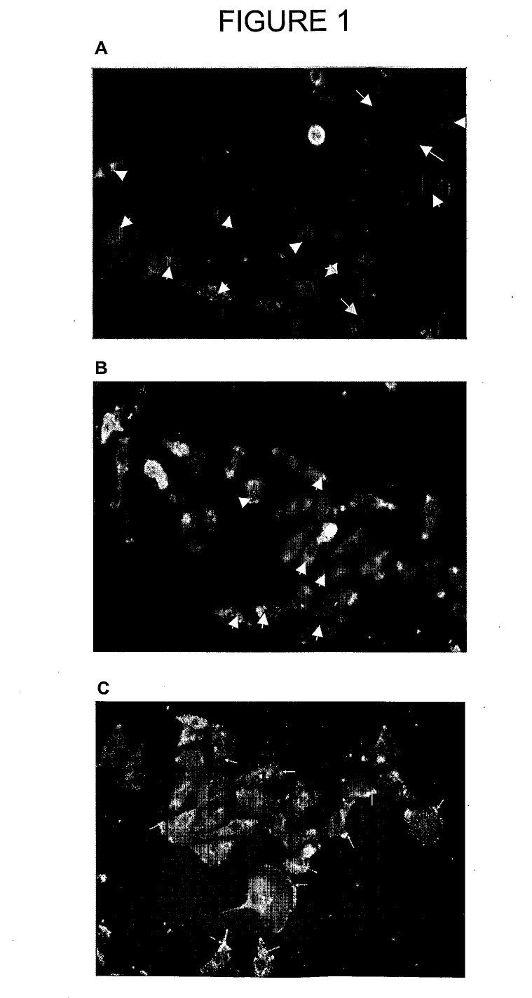

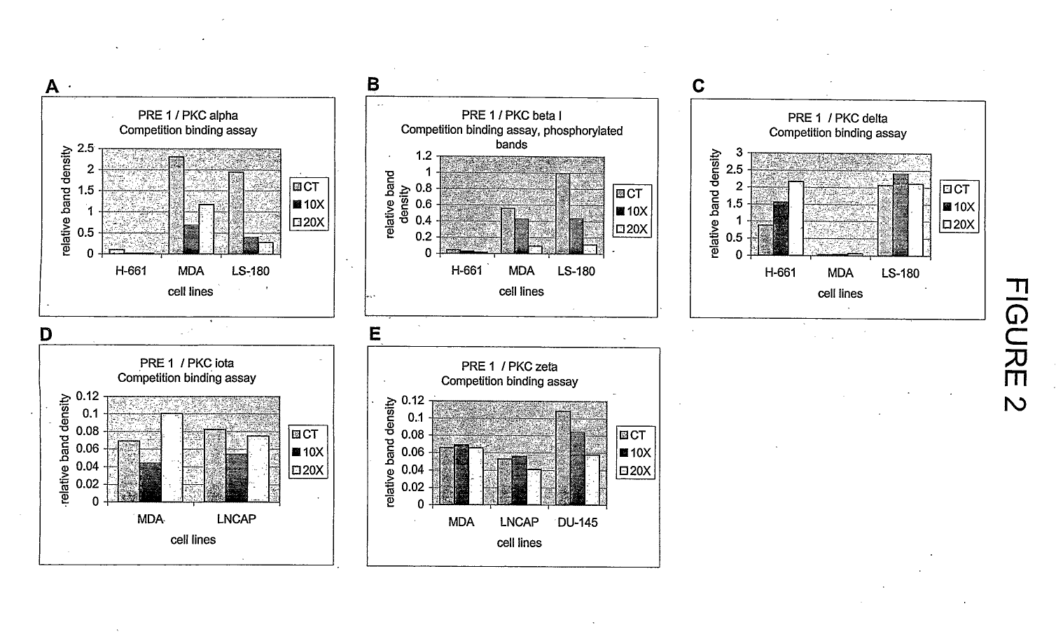

FIG. 1A shows the results for control, untreated cells. In most of the cells PKC-αcan be seen to be located in the cytoplasm, around the nucleus and at the plasma membrane (where it becomes activated).

FIG. 1B shows the results for cells treated with PRE 4. PKC-α can be seen to have accumulated in the cytoplasm of the treated cells as illustrated by the increased fluorescence intensity when compared to control cells (FIG. 1). PRE 4 treatment has thus prevented translocation of PKC-α to the membrane, which will also prevent activation of the enzyme.

FIG. 1C shows the results for cells treated with PRE 3. PKC-α can be seen to be located mostly on the membrane (white arrows) or around the nucleus (black arrows), suggesting that PRE 3 does not alter the subcellular localisation of PKC-α.

### Example 9

### In Vitro Competitive Binding Assays with Purified PKC Isoforms

Peptides PRE 1, 2, 3, 4, 5, 6, 7, 8, 9, 10, 11, 12 and 13 (see Table 3) were tested for their ability to interfere with binding of PKC isoform-specific antibodies to their target PKC isoforms using a competitive binding assay and purified PKC isoforms.

Procedure: Competitive binding was assessed using a 96 well plate ELISA-based assay as described below.

Greiner 96 well ELISA plates were coated with the appropriate PKC isoform diluted in PBSN (PBS with Calcium/Magnesium+0.05% Sodium azide w/w). 50 μL/well at 250 ng/mL was used. Control wells contained PBSN only. Plates were incubated overnight at 4° C.

The plates were washed 3 times with 200 μL of dH₂O per well, then 100 μL of blocking solution was added per well and the plate incubated for 1 h at 37° C. The washing steps with dH₂O were repeated and 50 μL of the appropriate PRE solution (PRE stock solution [20 mM in DMSO] diluted in PBSN) was added and the plate incubated for 1 h at room temperature.

After incubation, 5 μL of 1.1:400 anti-PKC (isoform specific antibody) was added per well (to provide a final concentration of 1:4000). For PKC-epsilon, 1.1:800 dilution was used and for PKC-zeta a 1.1:100 dilution was used due to high and low binding affinity, respectively. The plates were then incubated 1 h at room temperature, followed by washing with dH₂O as described above.

50 μL of 1:1000 dilution of Alkaline Phosphatase (AP) conjugated antibody (anti-rabbit for all except zeta, which was anti-goat) was then added and the plate incubated for 1 h at room temperature. Washing with dH₂O was then repeated. Finally, 100 μL of pNPP solution in pNPP buffer (2 mM MgCl₂, 100 mM Sodium bicarbonate in water, pH 9.8) was added to all wells and incubated for 8 minutes for α, βI, βII ε, and ζ, or for 15-20 mins for δ (until sufficient coloration is observed). The reaction was stopped by addition of Stop solution (2N NaOH) and the absorbance at 405 nm was read on a Galaxy Plate reader.

Controls were PKC without PRE; blank controls, and controls containing the final concentrations of DMSO used for solubilizing the PREs. The non-specific binding capacity related to DMSO was also measured.

The antibodies used were as follows: anti-PKC α (Cat. No. SC-208); anti-PKC βI (Cat. No. SC-209); anti-PKC βII (Cat. No. SC-210); anti-PKC δ (Cat. No. SC-213); anti-PKC ε (Cat. No. SC-214); anti-PKC ζ (Cat. No. SC-216-G) (all from Santa Cruz Biotechnology, Inc.). The AP-conjugated antibodies were obtained from Jackson Immuno Research Laboratories (West Grove, Pa.).

Calculations: The OD values measured at 405 nm represent the free PKC coated per well.

OD₄₀₅ PKC alone−Blank OD₄₀₅ of each sample=OD₄₀₅ coated isoform

(OD₄₀₅ of sample X/OD₄₀₅ coated isoform)×100=% n that represents the percentage of X binding compared to control.

100-% n measures the relative binding capacity of X towards the tested isoform.

The apparent binding capacity of the DMSO samples was then subtracted from X binding capacity.

Results: The results are shown in Tables 15-20. All measurements were made in triplicate and the values in the table represent the averaged calculated binding capacity values after subtraction of the DMSO apparent binding capacity (averaged from 12 values, respectively 10.80, 11.20 and 16.40 corresponding to the concentrations of DMSO used to dilute the tested isoform at 200, 100 and 50 μM respectively). The results allow for quantitative comparison of the binding capacity of each PRE towards an individual isoform within each table, but not among isoforms due to differences in sensitivity of the specific antibodies toward the secondary antibody. This applies particularly to PKC-delta. As noted above, the colour development duration was increased 2-3 times and, as a result, the OD₄₀₅ measurements may be overestimated for this isoform.

TABLE 15
Competitive Binding Assay with PKC-α

| | Concentration/μM | | |
|---|---|---|---|
| PRE | 200 | 100 | 50 |
| PRE 1 | 2.10 | 0 | 0.69 |
| PRE 4 | 22.8 | 21.9 | 6.46 |
| PRE 6 | 0 | 0 | 0 |
| PRE 3 | 22.61 | 26.5 | 17.47 |
| PRE 7 | 6.59 | 6.72 | 0 |
| PRE 8 | 0 | 0 | 0 |
| PRE 9 | 0 | 5.35 | 0 |
| PRE 10 | 0 | 0 | 0 |
| PRE 11 | 13.3 | 5.96 | 0 |
| PRE 12 | 0 | 5.16 | 3.29 |
| PRE 13 | 0 | 0 | 0 |
| PRE 5 | 0 | 0 | 0 |

TABLE 16
Competitive Binding Assay with PKC-βI

| | Concentration/μM | | |
|---|---|---|---|
| PRE | 200 | 100 | 50 |
| PRE 1 | 13.41 | 13.93 | 19.83 |
| PRE 4 | 7.46 | 15.71 | 6.02 |
| PRE 6 | 6.14 | 7.93 | 0 |
| PRE 3 | 23.04 | 22.49 | 26.01 |
| PRE 7 | 0 | 0 | 2.10 |
| PRE 8 | 0 | 0 | 0 |
| PRE 9 | 5.72 | 8.82 | 11.53 |
| PRE 10 | 0 | 4.35 | 3.77 |
| PRE 11 | 12.5 | 14.23 | 9.86 |
| PRE 12 | 0 | 0 | 0.79 |
| PRE 13 | 0 | 0 | 0 |
| PRE 5 | 0 | 0 | 0 |

TABLE 17
Competitive Binding Assay with PKC-βII

| | Concentration/μM | | |
|---|---|---|---|
| PRE | 200 | 100 | 50 |
| PRE 1 | 0 | 0 | 0 |
| PRE 4 | 1.18 | 13.53 | 2.69 |
| PRE 6 | 0 | 0 | 0.08 |
| PRE 3 | 3.72 | 0 | 0 |
| PRE 7 | 2.59 | 11.07 | 5.42 |
| PRE 8 | 0 | 0 | 1.11 |
| PRE 9 | 0 | 0.3 | 8.14 |
| PRE 10 | 0 | 0 | 0 |
| PRE 11 | 6.40 | 11.04 | 7.14 |
| PRE 12 | 0 | 0 | 4.55 |
| PRE 13 | 0 | 1.63 | 0 |
| PRE 5 | 0 | 0 | 0 |

TABLE 18
Competitive Binding Assay with PKC-δ

| | Concentration/μM | | |
|---|---|---|---|
| PRE | 200 | 100 | 50 |
| PRE 1 | 49.43 | 45.20 | 35.71 |
| PRE 4 | 17.31 | 30.7 | 29.25 |
| PRE 6 | 13.96 | 13.15 | 17.46 |
| PRE 3 | 57.01 | 56.01 | 48.27 |
| PRE 7 | 0 | 0 | 3.06 |
| PRE 8 | 6.68 | 3.38 | 6.96 |
| PRE 9 | 9.95 | 19.27 | 16.23 |
| PRE 10 | 9.47 | 0 | 0 |
| PRE 11 | 18.74 | 21.48 | 34.06 |
| PRE 12 | 23.49 | 1.02 | 2.26 |
| PRE 13 | 17.85 | 0.79 | 0 |
| PRE 5 | 4.31 | 0 | 0.20 |

TABLE 19
Competitive Binding Assay with PKC-ε

| | Concentration/μM | | |
|---|---|---|---|
| PRE | 200 | 100 | 50 |
| PRE 1 | 0.83 | 5.47 | 19.84 |
| PRE 4 | 2.58 | 0 | 0 |
| PRE 6 | 0 | 0.46 | 2.61 |
| PRE 3 | 30.98 | 31.51 | 33.99 |
| PRE 7 | 3.78 | 13.99 | 8.04 |
| PRE 8 | 0 | 0 | 0 |
| PRE 9 | 0 | 8.53 | 16.35 |
| PRE 10 | 1.75 | 0 | 0.91 |
| PRE 11 | 9.84 | 13.02 | 2.25 |
| PRE 12 | 0 | 0.28 | 10.44 |
| PRE 13 | 0.85 | 0 | 0 |
| PRE 5 | 1.80 | 0 | 0 |

TABLE 20
Competitive Binding Assay with PKC-ζ

| | Concentration/μM | | |
|---|---|---|---|
| PRE | 200 | 100 | 50 |
| PRE 1 | 0 | 0 | 4.11 |
| PRE 4 | 5.85 | 0 | 0 |
| PRE 6 | 0 | 0 | 0 |
| PRE 3 | 29.15 | 23.94 | 14.00 |
| PRE 7 | 1.54 | 0 | 1.83 |
| PRE 8 | 0 | 0 | 0 |
| PRE 9 | 0 | 0 | 0 |
| PRE 10 | 0 | 0 | 0 |
| PRE 11 | 0 | 0 | 0 |
| PRE 12 | 0.33 | 8.77 | 4.20 |
| PRE 13 | 4.29 | 0 | 0 |
| PRE 5 | 0 | 0 | 0 |

As can be seen from Tables 15-20 above, PKC-α is targeted most strongly by PRE 3 and PRE 4; PKC-βI is targeted most strongly by PRE 1 and PRE 3; PKC-βII is targeted most strongly by PRE 9 (at 50 μM); PKC-δ is targeted most strongly by PRE 1, PRE 3, PRE 11 and PRE 4; PKC-ε is targeted most strongly by PRE 3, and PKC-ζ is targeted by PRE 3 only.

PRE 4 demonstrates specificity for PKC-α with the exception of some possible affinity for PKC-δ, which may be overestimated for reasons outlined above.

PRE 3 appears to be a “universal” PKC targeting peptide, with the exception of the PKC-βII isoform. This is of interest since the discrimination between the two isoforms PKC-βI and PKC-βII is traditionally difficult because they result from alternative splicing.

### Example 10

### In Vitro Competitive Binding Assays Using Cell Extracts

Binding efficiency and specificity of the peptides PRE 2, 3, 4, 5, 6, 7, 8, 9, 10, 11, 12 and 13 (see Table 4) was also tested using cytoplasmic extracts from different cell lines expressing appropriate PKC isoforms as described for Example 1. Briefly, the protein cytoplasmic extracts were separated by electrophoresis and transferred onto nitrocellulose membranes by standard Western blotting procedures. The bands on the Western blots were detected with matching primary antibodies and alkaline phosphatase conjugated secondary antibody. The bands were then scanned and the relative density measurements obtained.

This procedure to assess the binding characteristics of the PREs is based on competition binding of the PRE with the primary anti-isoform PKC specific antibodies. The lower the measured band density, the greater the binding of the PRE to the PKC isoform. The PREs were added at 10 and 20 times (10×, 20×) the primary antibody concentration, according to classical competition between antigenic peptide and primary antibody. The following cell lines (obtained from the ATCC) were used: H661—NCI human lung carcinoma NSCLC; MDAMB231—human highly invasive breast cancer cell line from pleural effusion; LS180—human colon adenocarcinoma; LnCAP—human prostate adenocarcinoma; CCD16Lu—human lung fibroblasts; Du145—human prostate carcinoma brain metastasis, and T24—human bladder carcinoma.

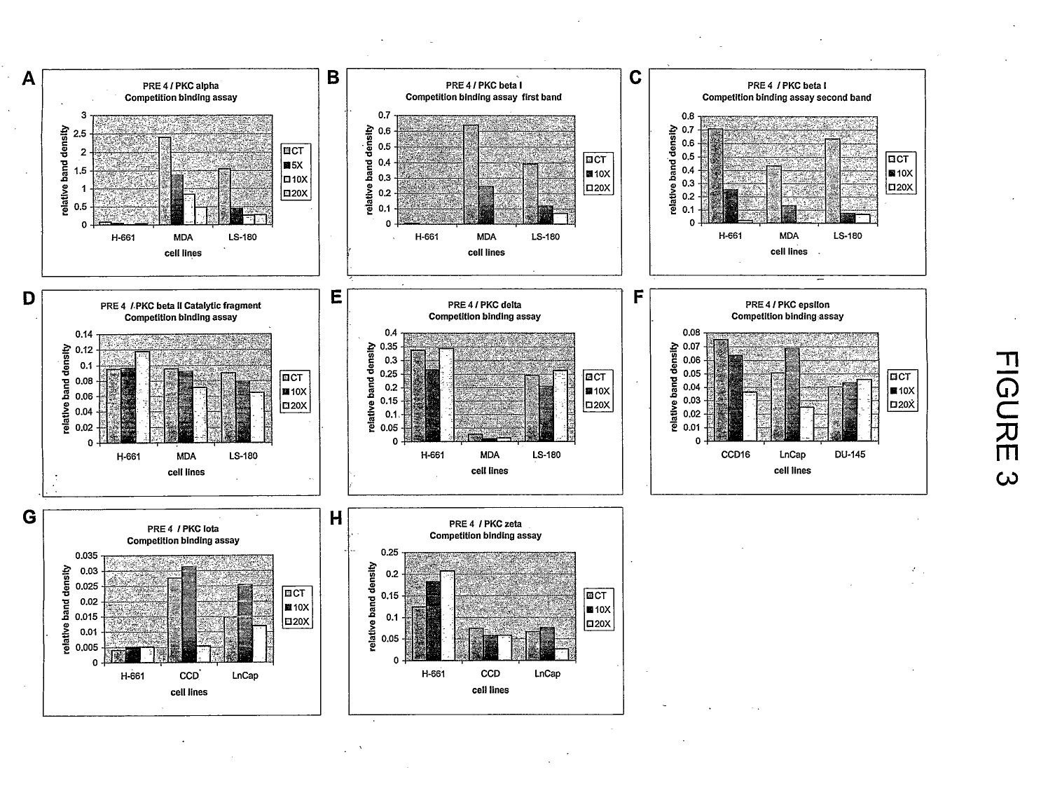

The results are shown in FIGS. 2-13. As can be seen from the Figures, the controls (without PRE) were sometimes lower in density than the 10× challenged protein bands. In these cases, the 20× band density figures relative to control and 10× values were reliable. The results are also summarised in Table 20 below.

FIG. 2 shows the effect of PRE 1 on (A) PKC-α in H661, MDAMB231 and LS180 cells; (B) PKC-βI in H661, MDAMB231 and LS180 cells, (C) PKC-δ in H661, MDAMB231 and LS180 cells, (D) PKC-ι in MDAMB231 and LnCAP cells and (E) PKC-ζ in MDAMB231, LnCAP and Du-145 cells.

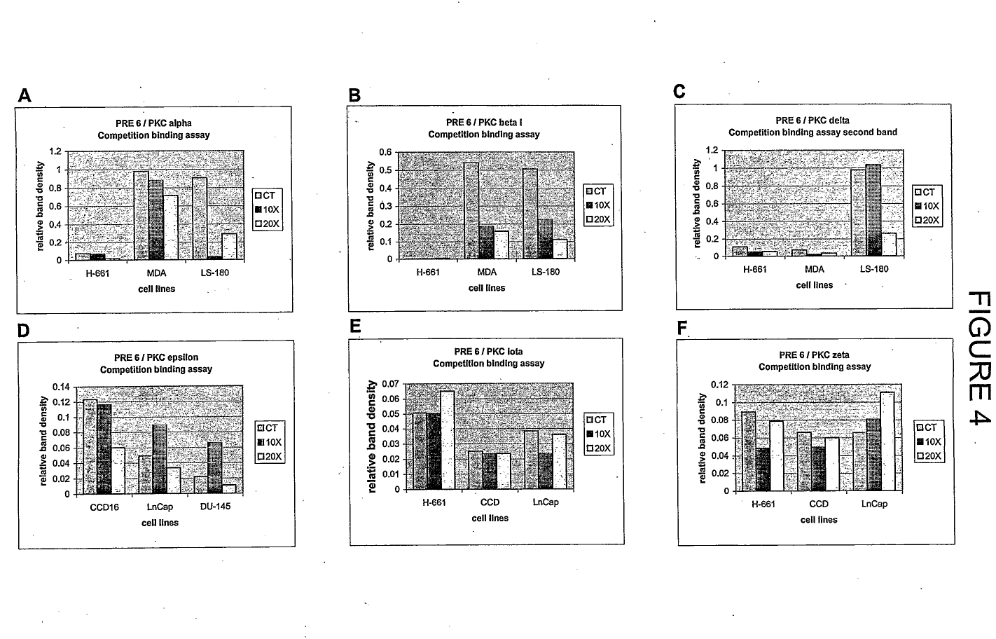

FIG. 3 shows the effect of PRE 4 on (A) PKC-α in H661, MDAMB231 and LS180 cells; (B) PKC-βI (first band on Western blot) in H661, MDAMB231 and LS180 cells, (C) PKC-βI (second band on Western blot) in H661, MDAMB231 and LS180 cells, (D) PKC-βII (catalytic fragment) in H661, MDAMB231 and LS180 cells, (E) PKC-δ in H661, MDAMB231 and LS180 cells, (F) PKC-ε in CCD16, LnCAP and Du-145 cells, (G) PKC-t in H661, CCD16 and LnCAP cells and (H) PKC-ζ in H661, CCD16 and LnCAP cells.

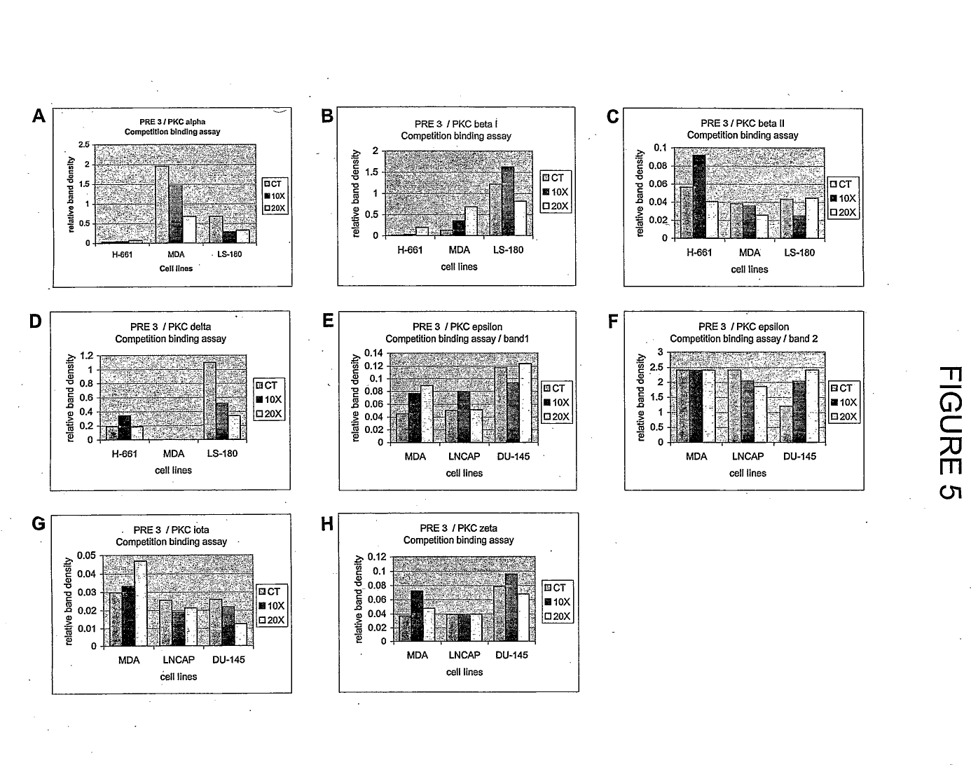

FIG. 4 shows the effect of PRE 6 on (A) PKC-α in H661, MDAMB231 and LS180 cells; (B) PKC-βI in H661, MDAMB231 and LS180 cells, (C) PKC-δ in H661, MDAMB231 and LS180 cells, (D) PKC-ε in CCD16, LnCAP and Du-145 cells, (E) PKC-ι in H661, CCD16 and LnCAP cells and (F) PKC-ζ in H661, CCD16 and LnCAP cells.

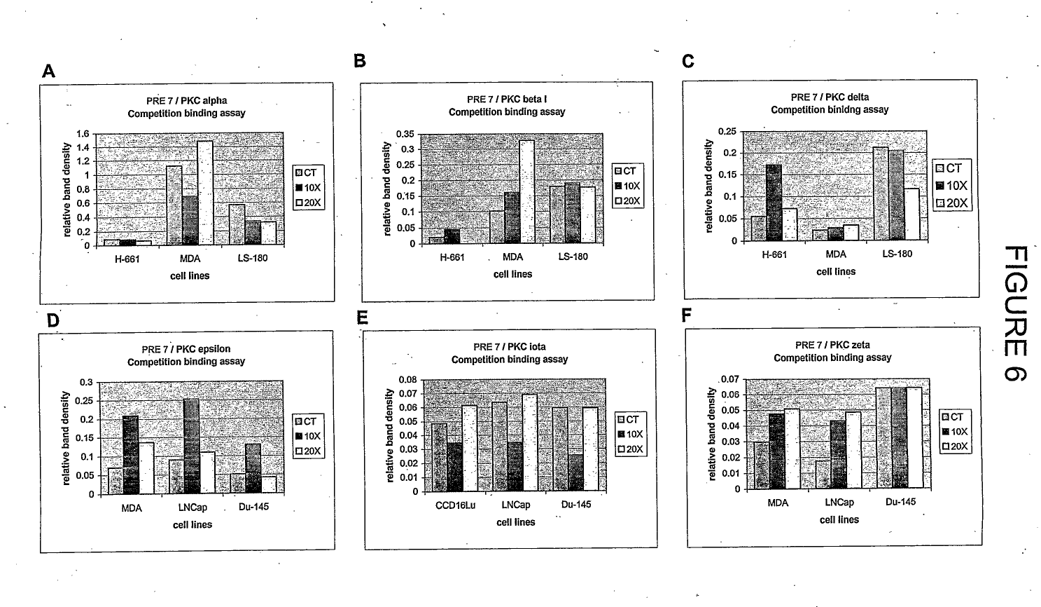

FIG. 5 shows the effect of PRE 3 on (A) PKC-α in H661, MDAMB231 and LS180 cells; (B) PKC-βI in H661, MDAMB231 and LS180 cells, (C) PKC-βII in H661, MDAMB231 and LS180 cells, (D) PKC-δ in H661, MDAMB231 and LS180 cells, (E) PKC-ε (Band 1 in Western blot) in MDAMB231, LnCAP and Du-145 cells, (F) PKC-ε (Band 2 in Western blot) in MDAMB231, LnCAP and Du-145 cells, (G) PKC-ι in MDAMB231, LnCAP and Du-145 cells and (E) PKC-ζ in MDAMB231, LnCAP and Du-145 cells.

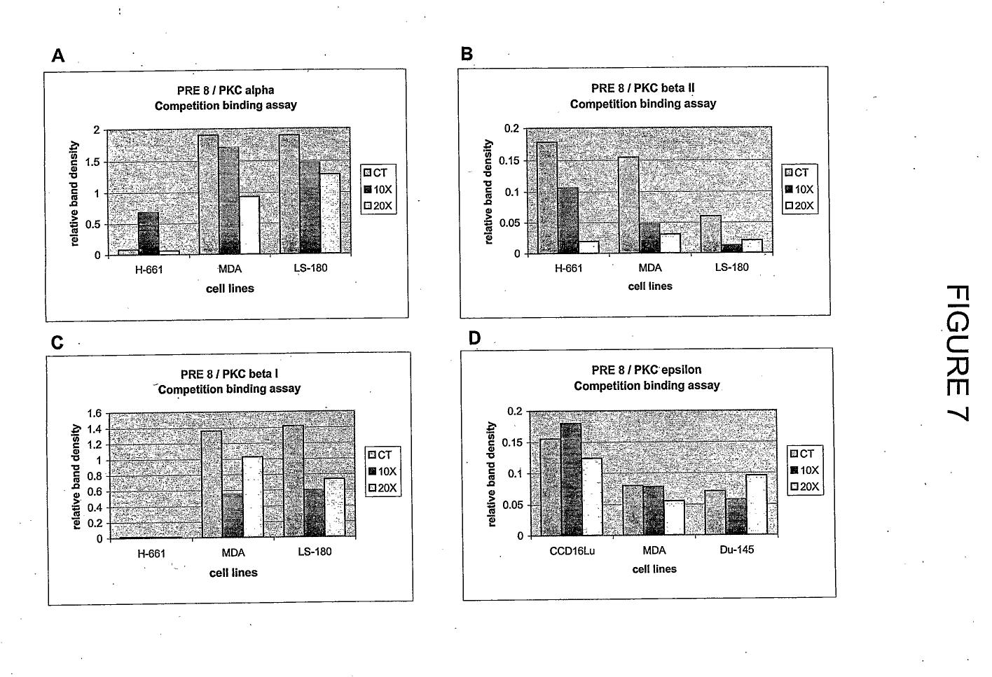

FIG. 6 shows the effect of PRE 7 on (A) PKC-α in H661, MDAMB231 and LS180 cells; (B) PKC-βI in H661, MDAMB231 and LS180 cells, (C) PKC-δ in H661, MDAMB231 and LS180 cells, (D) PKC-ε in MDAMB231, LnCAP and Du-145 cells, (E) PKC-ι in CCD16, LnCAP and Du-145 cells and (E) PKC-ζ in MDAMB231, LnCAP and Du-145 cells.

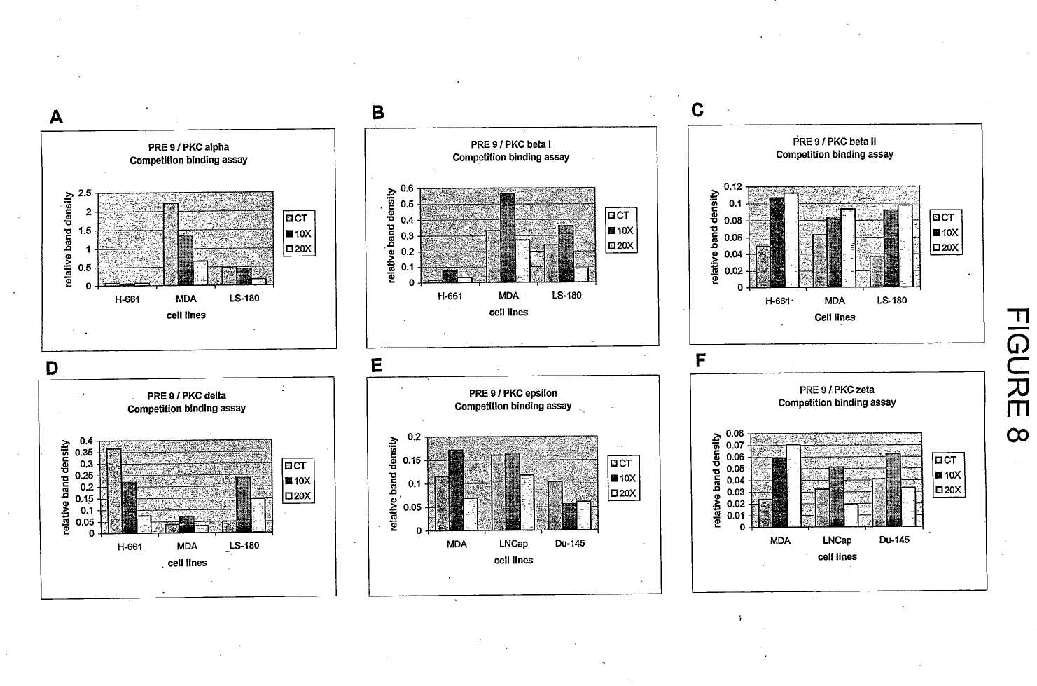

FIG. 7 shows the effect of PRE 8 on (A) PKC-α in H661, MDAMB231 and LS180 cells; (B) PKC-βII in H661, MDAMB231 and LS180 cells, (C) PKC-βI in H661, MDAMB231 and LS180 cells and (D) PKC-ε in CCD16, MDAMB231 and Du-145 cells.

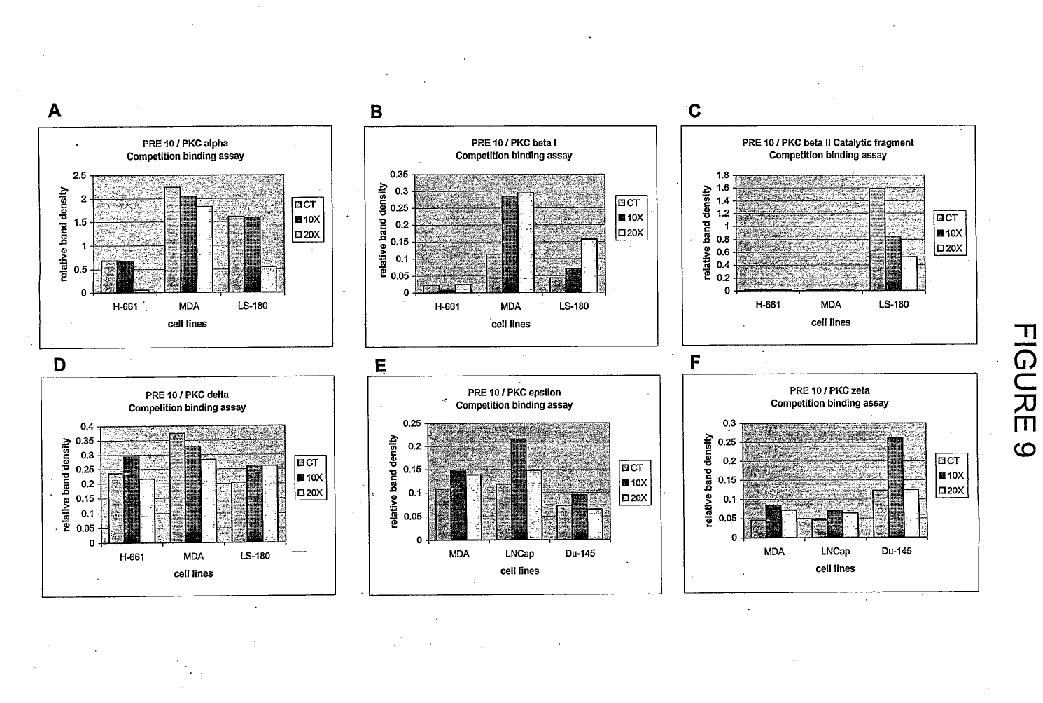

FIG. 8 shows the effect of PRE 9 on (A) PKC-α in H661, MDAMB231 and LS180 cells; (B) PKC-βI in H661, MDAMB231 and LS180 cells, (C) PKC-βII in H661, MDAMB231 and LS180 cells, (D) PKC-δ in H661, MDAMB231 and LS180 cells, (E) PKC-ε in MDAMB231, LnCAP and Du-145 cells, and (F) PKC-ζ in MDAMB231, LnCAP and Du-145 cells.

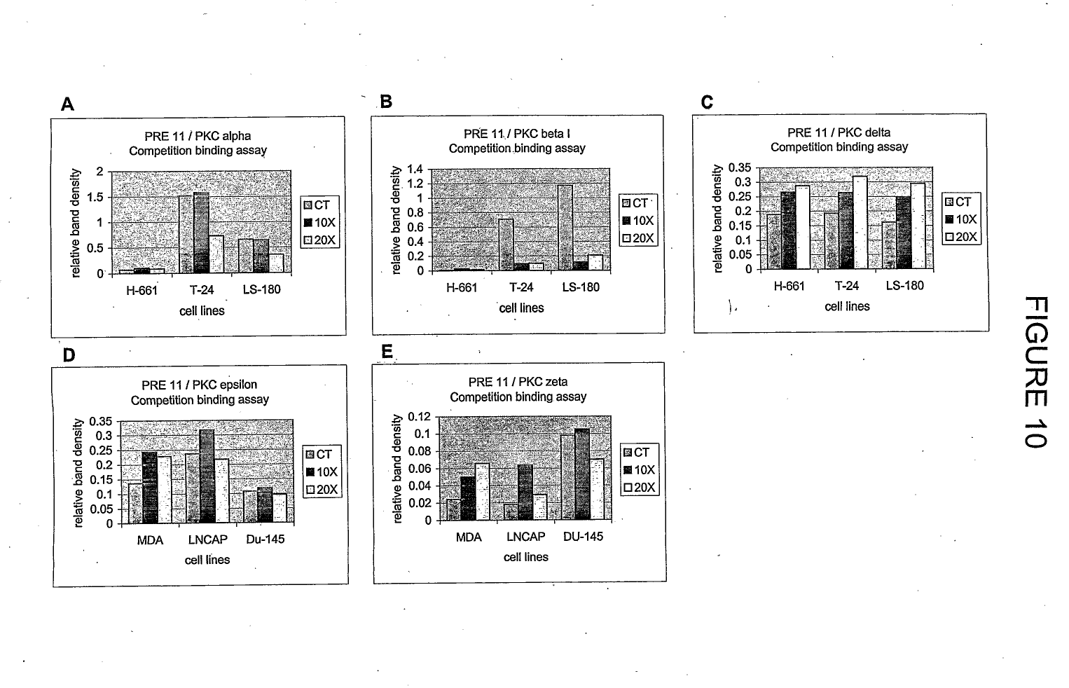

FIG. 9 shows the effect of PRE 10 on (A) PKC-α in H661, MDAMB231 and LS180 cells; (B) PKC-βI in H661, MDAMB231 and LS180 cells, (C) PKC-βII (catalytic fragment) in H661, MDAMB231 and LS180 cells, (D) PKC-δ in H661, MDAMB231 and LS180 cells, (E) PKC-ε in MDAMB231, LnCAP and Du-145 cells, and (F) PKC-ζ in MDAMB231, LnCAP and Du-145 cells.

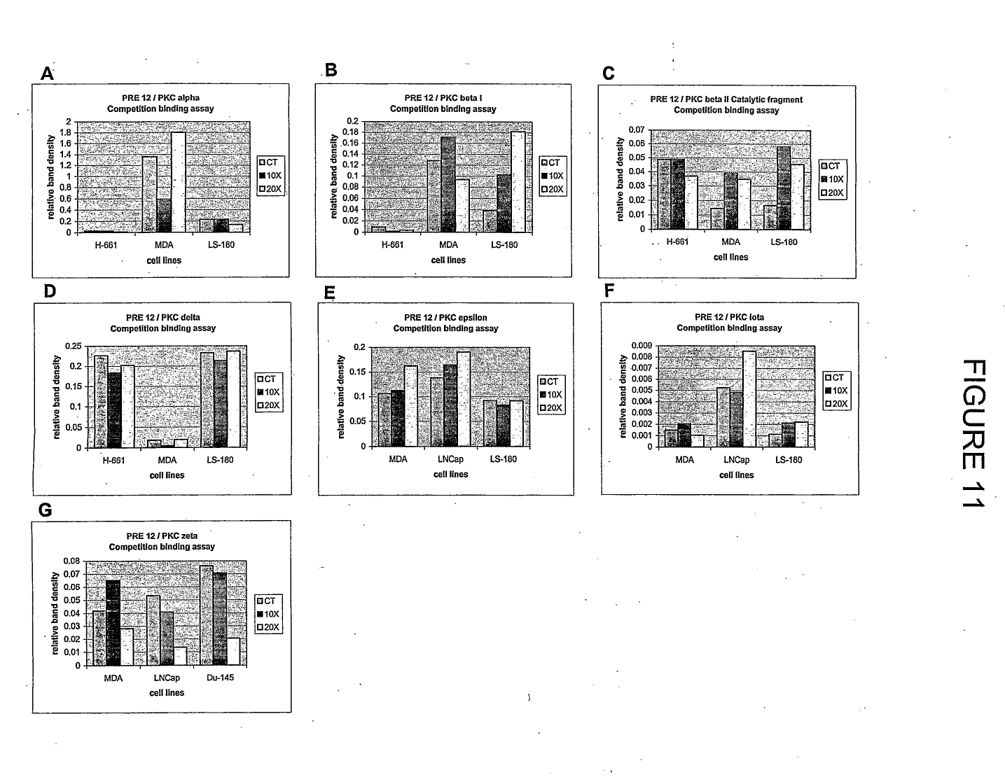

FIG. 10 shows the effect of PRE 11 on (A) PKC-α in H661, T24 and LS180 cells; (B) PKC-βI in H661, T24 and LS180 cells, (C) PKC-δ in H661, 724 and LS180 cells, (D) PKC-ε in MDAMB231, LnCAP and Du-145 cells, and (E) PKC-ζ in MDAMB231, LnCAP and Du-145 cells.

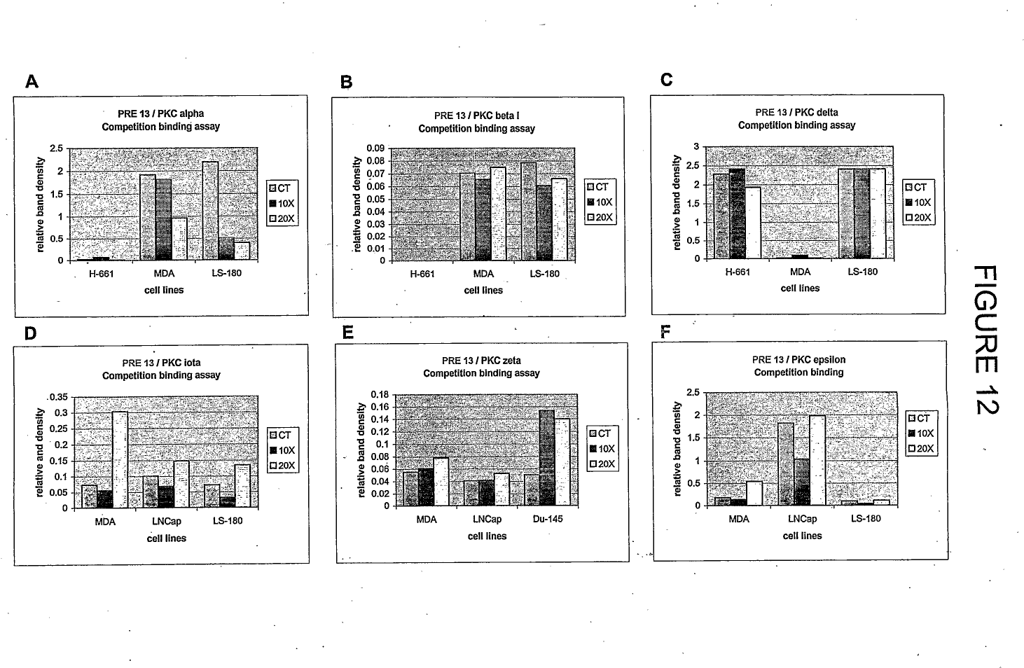

FIG. 11 shows the effect of PRE 12 on (A) PKC-α in H661, MDAMB231 and LS180 cells; (B) PKC-βI in H661, MDAMB231 and LS180 cells, (C) PKC-βII (catalytic fragment) in H661, MDAMB231 and LS180 cells, (D) PKC-δ in H661, MDAMB231 and LS180 cells, (E) PKC-ε in MDAMB231, LnCAP and LS180 cells, (F) PKC-ι in MDAMB231, LnCAP and LS180 cells and (G) PKC-ζ in MDAMB231, LnCAP and Du-145 cells.

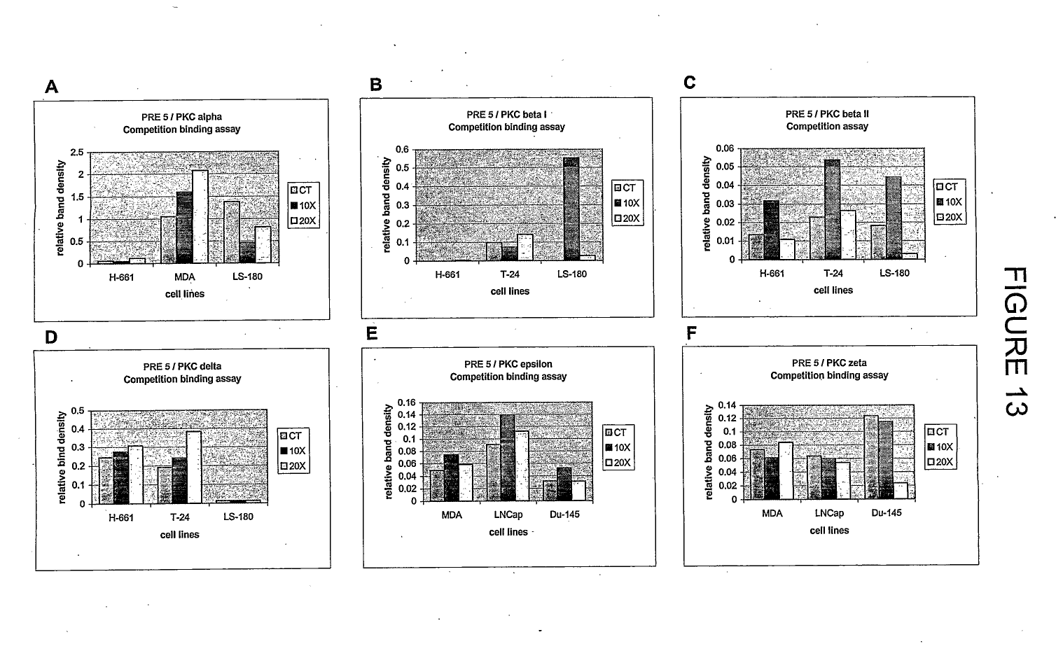

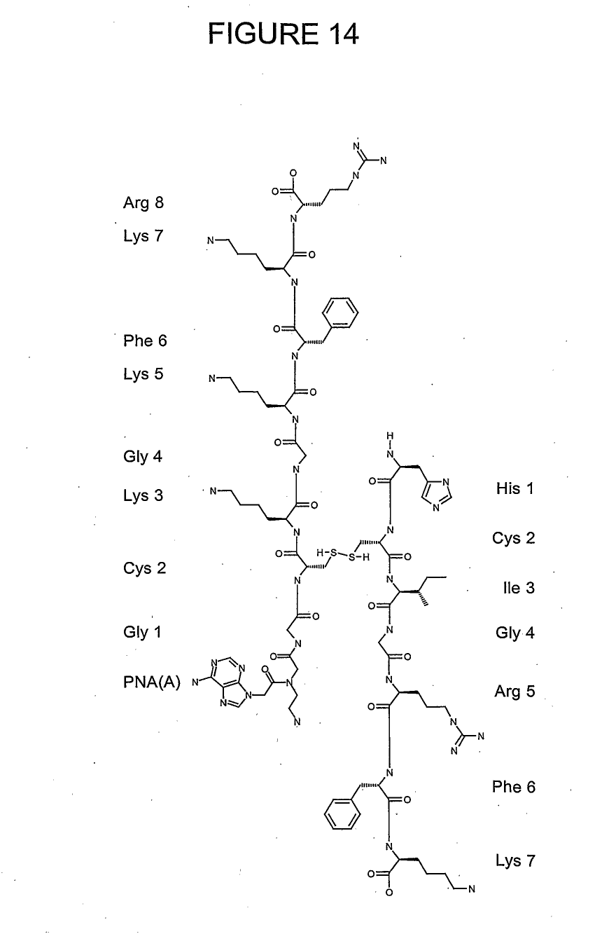

FIG. 12 shows the effect of PRE 13 on (A) PKC-α in H661, MDAMB231 and LS180 cells; (B) PKC-βI in H661, MDAMB231 and LS180 cells, (C) PKC-δ in H661, MDAMB231 and LS180 cells, (D) PKC-t in MDAMB231, LnCAP and LS180 cells, (E) PKC-ζ in MDAMB231, LnCAP and Du-145 cells, and (F) PKC-ε in MDAMB231, LnCAP and LS180 cells.

FIG. 13 shows the effect of PRE 5 on (A) PKC-α in H661, MDAMB231 and LS180 cells; (B) PKC-βI in H661, T24 and LS180 cells, (C) PKC-βII in H661, T24 and LS180 cells, (D) PKC-δ in H661, T24 and LS180 cells, (E) PKC-ε in MDAMB231, LnCAP and Du-145 cells, and (F) PKC-ζ in MDAMB231, LnCAP and Du-145 cells.

The results obtained in this Example with cell lysates correlate in many respects with the results obtained using the purified isoforms in Example 10. Notably, PRE 4 is shown in this Example to have a strong affinity for PKC-α, as was the case with the purified isoform results in Example 9. Some discrepancies, however, also occur. Specifically, using cell extracts, PRE 4 showed a good affinity for PKC-βI and none for PKC-δ, whereas using purified isoforms, PRE 4 showed an affinity for PKC-δ. This inconsistency may originate from the fact that the enzymes were denatured and linearized on the Western blots, while the purified enzymes retained their 3-dimensional configurations. In addition, the relative concentrations of the enzymes versus the PRE concentrations can be better controlled when purified enzymes are used and a difference in this enzyme:PRE ratio between the two techniques may introduce differences in the sensitivity of the experiments. Finally, there may be some binding of the PREs to unknown proteins which are present but undetected on the Western blots and which are expressed differently in selective cell lines.

The results of the two sets of experiments reported in Examples 9 and 10 are summarised in Table 21.

TABLE 21
Affinity of PRE 2-13 for Various PKC Isoforms*

| | | PKC Isoform | | | | | | |
|---|---|---|---|---|---|---|---|---|
| | | α | βI | βII | δ | ε | ι | ζ |
| PRE 1 | Purified | — | H | — | H | — | ND | — |
| | isoform | | | | | | | |
| | Cell extract | H | H | ND | — | ND | — | L |
| PRE 4 | Purified | H | L | L | H | — | ND | — |
| | isoform | | | | | | | |
| | Cell extract | H | H | L | — | H/L | M/L | — |
| PRE 6 | Purified | — | L | — | L | — | ND | — |
| | isoform | | | | | | | |
| | Cell extract | H/L | H | ND | M/H | M | — | — |
| PRE 3 | Purified | H | H | — | H | H | ND | H |
| | isoform | | | | | | | |
| | Cell extract | H | L | L | H | L | L | — |
| PRE 7 | Purified | L | — | — | — | L | ND | — |
| | isoform | | | | | | | |
| | Cell extract | L | — | ND | L | — | — | — |
| PRE 8 | Purified | — | — | — | — | — | ND | — |
| | isoform | | | | | | | |
| | Cell extract | M/L | ND | H | L | L | ND | ND |
| PRE 9 | Purified | — | L | H | L | L | ND | — |
| | isoform | | | | | | | |
| | Cell extract | M/L | M | — | H | M/L | ND | M/L |
| PRE 10 | Purified | — | — | L | — | — | ND | — |
| | isoform | | | | | | | |
| | Cell extract | H/L | M | — | H | M/L | ND | M/L |
| PRE 11 | Purified | L | — | — | H | L | ND | — |
| | isoform | | | | | | | |
| | Cell extract | H/L | H | ND | — | — | ND | — |
| PRE 12 | Purified | — | — | — | L | — | ND | — |
| | isoform | | | | | | | |
| | Cell extract | — | — | L | — | — | — | M/H |
| PRE 13 | Purified | — | — | — | L | — | ND | — |
| | isoform | | | | | | | |
| | Cell extract | M/H | — | ND | — | — | — | — |
| PRE 5 | Purified | — | — | — | L | — | ND | — |
| | isoform | | | | | | | |
| | Cell extract | M | H | H | — | L | ND | L/H |

*Legend:
— = no detectable affinity
L = Low to very low affinity at 10X or 20X or both PRE concentrations in at least one cell line in the cell extract assay; Low affinity in purified isoform assay.
M = Moderate affinity at 10X or 20X or both PRE concentrations in at least one cell line in the cell extract assay.
H = High affinity at 10X or 20X or both PRE concentrations in at least one cell line in the cell extract assay; High affinity in purified isoform assay.
ND = not determined.

### Example 11

### Preparation of Protein Kinase Inhibiting Compounds PKI 1 to PKI 10

Compounds PKI 1 to PKI 10 (shown below) were synthesized using standard procedures as represented by the following protocol for the preparation of compound PKI 3. Compound PKI 4, which does not contain a PNA moiety was synthesized on an Applied Biosystems Pioneer Peptide Synthesizer following the protocol provided by the manufacturer.

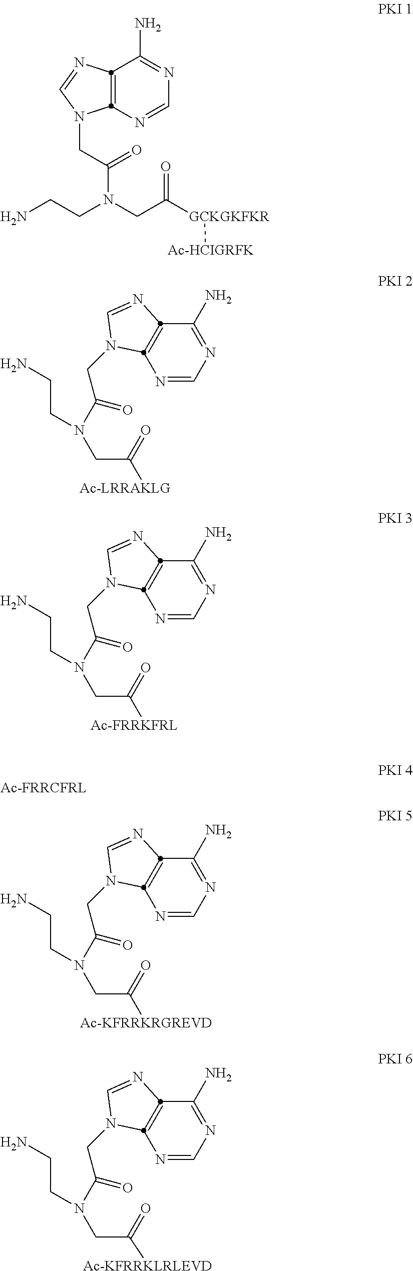

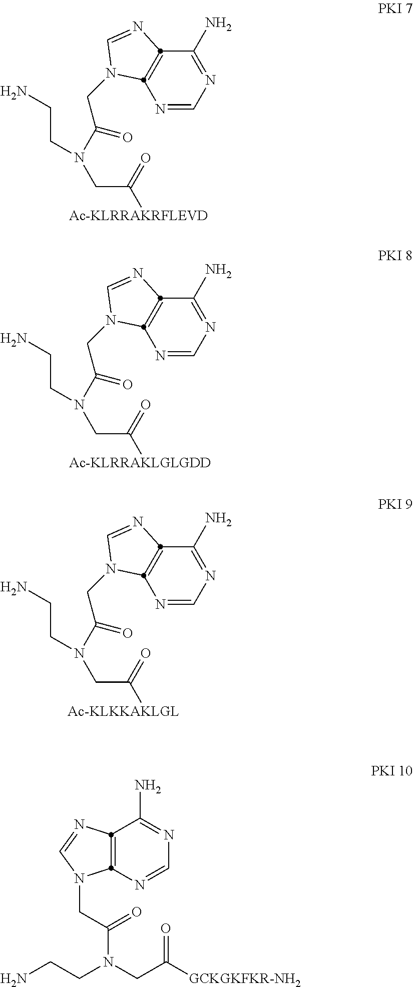

### Preparation of Compound PKI 3

The peptide chain FRRKFRL was synthesized on an Applied Biosystems Pioneer Peptide Synthesizer following the protocol provided by the manufacturer and employing Lys in which the side chain is protected with the amine protecting group ivDde.

After the peptide chain was synthesized, the ivDde protecting group was removed by washing the resin with DMF, isopropanol and dichloromethane, allowing the resin to dry for 20 min, then washing for 30 min with 2% Hydrazine (in DMF). The resin was then washed again with DMF, isopropanol and dichloromethane and allowed to dry.

Adenine peptide nucleic acid (PNA(Bhoc)) was coupled to the side chain of the Lys residue in the peptide chain by shaking the resin in DMF solvent with 2 eq activator HBTU/HOBt, 2 eq DIPEA and 2 eq PNA(Bhoc). After 12 hrs, the resin was washed and then submitted to a de-protection step to remove the Fmoc from the PNA by shaking the resin with 20% piperidine/DMF for 6 hrs. Finally, the peptide was cleaved from the resin using standard protocols. After filtering, the peptide was dissolved in H₂O (0.1% TFA) and purified by column chromatography.

### Mass Spectrometry

The structure of the compounds was confirmed by electrospray mass spectrometry as follows. For compounds comprising a PNA moiety, mass spectral analysis was performed before and after addition of the PNA moiety.

Analysis by mass spectrometry was performed on a VG Quattro I (Fison, UK) mass spectrometer equipped with pneumatically-assisted electrospray ionisation source, operating in positive mode. The solvent system was 1:1 acetonitrile:water with 0.2% formic acid with a flow rate of 15 μl per minute. The source temperature was set at 85° C., an electrospray capillary was set at 3.5 kV with a cone voltage set at 20V. Data were collected in continuum mode between 200-2000 m/z with sweep time of 10 seconds. Spectra obtained for each compound were combinations of 5 consecutive scans and background subtraction. The respective mass of each compound was calculated using Transform mode in MassLynx 3.5 software.

The respective calculated and measured masses for compounds PKI 1 to PKI 10 are shown in Table 22.

TABLE 22
Calculated and Measured Mass for Compounds PKI 1-10

| Compound | Calculated Mass | Measured Mass |
|---|---|---|
| PKI 1 | 2138.35 | 2138 |
| PKI 2 | 1129.15 | 1129 |
| PKI 3 | 1339.14 | 1339 |
| PKI 4 | 1038.16 | 1038 |
| PKI 5 | 1762.73 | 1763 |
| PKI 6 | 1775.81 | 1776 |
| PKI 7 | 1846.83 | 1847 |
| PKI 8 | 1657.58 | 1658 |
| PKI 9 | 1314.47 | 1314 |
| PKI 10 | 1239.15 | 1239 |

### Example 12

### In Vitro Inhibition of Purified PKC-Alpha with Compounds PKI 1, PKI 2 and PKI 3

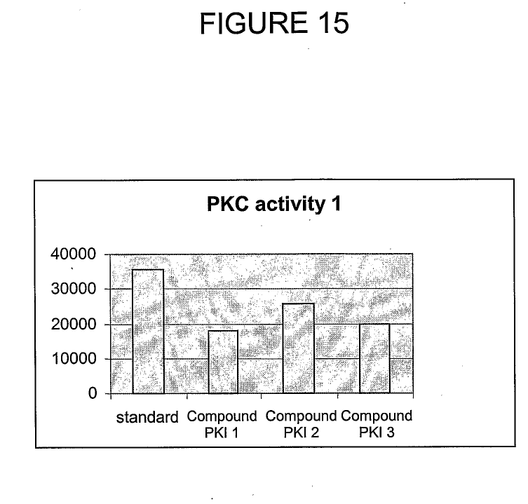

The ability of each of compounds PKI 1, PKI 2 and PKI 3 to inhibit the activity of commercially purified PKC-alpha activity was tested. PKC-alpha was obtained from Upstate Cell Signalling Solutions #14-484 (Lake Placid N.Y.). The PKC-alpha activity assays were performed using the IQ™ PKC assay kit, a kit from Pierce Biotechnology (Rockford, Ill.), according to manufacturer instructions. Compounds 1, 2 and 3 were used at a concentration of 10 μg per assay. As shown in FIG. 15, all three compounds showed inhibitory activity. The relative activities are expressed in arbitrary fluorescence intensity units using Galaxy plate reader (BMG LabTech, GmbH, Offenburg/Germany). The assays were run in duplicate and repeated twice. The values shown in FIG. 15 are the average of 4 assays; “Standard” indicates the control reaction in the absence of any inhibitor.

### Example 13

### In Vitro Inhibition of PKC-Alpha in Cell Lysates with Compounds PKI 1, PKI 2 and PKI 3

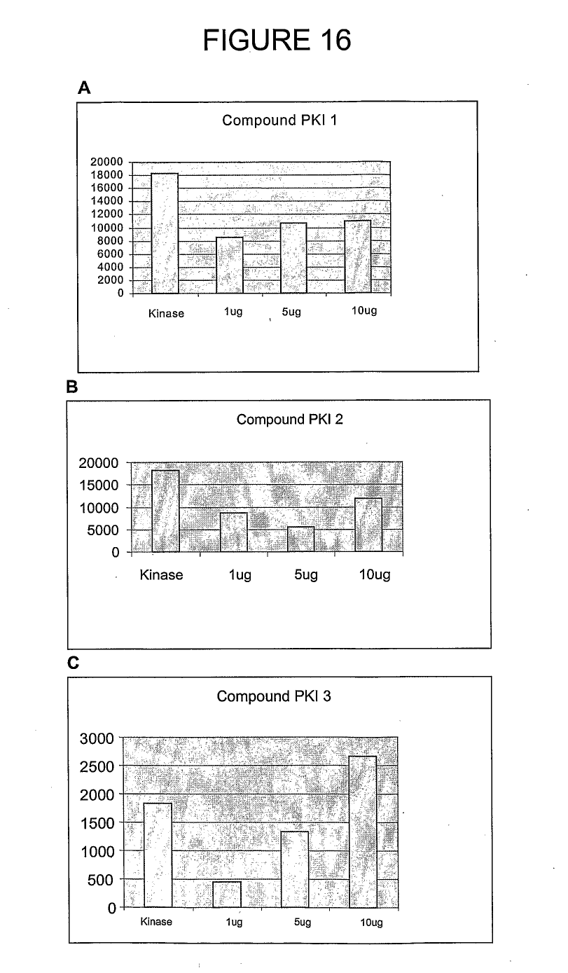

Compounds PKI 1, PKI 2 and PKI 3 were tested as described above (Example 12) except that the source of the PKC-alpha enzyme was a cell lysate from neuroblastoma IMR-32 cells that had been grown for 48 h. The cells (1×10⁷) were frozen at −80° C. under a film (400 μl) of RIPA buffer supplemented with a cocktail of protease inhibitors and orthovanadate. The extract was thawed and centrifuged at 14,000×g for 10 min in a refrigerated centrifuge. The clear supernatant was used as the source of enzyme. The results are shown in FIG. 16; “Kinase” represents the activity of the untreated extract. Concentrations of the PKI compounds are as indicated.

Both compound PKI 1 (FIG. 16A) and PKI 2 (FIG. 16B) exhibited good activity but no dose response was observed, suggesting that the compounds may be active at lower concentration and also that other cellular kinases may compete for the compound.

Compound PKI 3 (FIG. 16C) exhibited good activity at 1 μg suggesting that it is active at lower concentration. However, compound PKI 3 showed a “reverse dose response” when assayed against the cell lysate, which contrasts with the observation that compound PKI 3 drastically inhibits the standard purified PKC-alpha at the dose 10 μg (FIG. 15).

### Example 14

### In Vitro Inhibition of Cancer Cell Proliferation with Compounds PKI 1, PKI 2 and PKI 3

The ability of compounds PKI 1, PKI 2 and PKI 3 to inhibit cancer cell proliferation was tested in vitro using the human neuroblastoma cell line IMR-32. Monolayer cell cultures were trypsinized and the test compound was added at the doses indicated in Table 24 and internalized by pinocytic endocytosis using Influx TM pinocytic cell-loading reagent, a kit from Molecular Probes (Eugene, Oreg.) following the manufacturer's recommendations. The indicated doses refer to the concentration in the loading medium, which was used on 1×10⁶ cells in a 10 μl volume, i.e. contained 10 μg to 100 ug of test compound that provides 10 fg to 100 fg per cell. It is worth noting, however, that only a small proportion of the compound is internalized using this technique, so the actual dose may be lower. The cells were treated on day 0 of the experiment. The cells loaded with test compound were cultured in 96 well plates (5,000 cells in 100 μl per well), and the proliferation was monitored over 3 consecutive days. The increase in cell population was quantified using a Hoechst reagent-based assay (modified from Rao and Otto, 1992, Analytical Biochem. 207:186-192) that measures the total DNA of the population. Fluorescence was measured using a Millipore CytoFluor 2350 plate reader (excitation at 360 nm and emission at 460 nm). The measurements were obtained as relative fluorescence intensity, a value that is directly correlated to the total number of cells. The results are shown in Table 23. Data are expressed as a percentage of matching controls that were supplemented with culture medium alone.

There was a clear dose response in the inhibition of the IMR-32 cells with compound PKI 1. As can be seen from the results in Table 23, there is an inverse correlation between the concentration of compound PKI 1 and the level of inhibition indicating that the compound is active at very low doses, but that once the compound saturates the cells, it may be competed for by a number of different protein kinases.

TABLE 23
Inhibition of IMR-32 Cancer Cell Proliferation with
Compounds PKI 1, 2 and 3

| | | % Inhibition | | |
|---|---|---|---|---|
| Compound | | 24 h | 48 h | 72 h |
| PKI 1 | 1.0 mg/mL | 54 | 58 | 77 |
| | 2.5 mg/mL | 36 | 39 | 42 |
| | 5.0 mg/mL | 12 | 22 | 36 |
| | 10.0 mg/mL | 8 | 17 | 6 |
| PKI 2 | 1.0 mg/mL | 49 | 51 | 10 |
| | 2.5 mg/mL | 29 | 21 | 26 |
| | 5.0 mg/mL | 31 | 20 | 17 |
| | 10.0 mg/mL | 31 | 29 | 21 |
| PKI 3 | 1.0 mg/mL | 70 | 72 | 72 |
| | 2.5 mg/mL | 26 | 29 | 14 |
| | 5.0 mg/mL | 29 | 31 | 22 |
| | 10.0 mg/mL | 26 | 30 | 22 |

### Example 15

### In Vitro Inhibition of Protein Kinase C Isoforms with Compounds PKI 1 to 10

The inhibitory effect of the PKI compounds 1 to 10 on purified commercially available isoforms representative of the 3 classes of PKC: cPKC (alpha, betaI/II) nPKC (delta and epsilon) aPKC (zeta) was assayed using the PepTag® Non-Radioactive PKC Assay (Promega, Madison, Wis.). The zeta isoform was tested with this assay although its affinity for the substrate provided in the kit was relatively low. The amount of enzyme in the reaction mixture for the PKC zeta assays was multiplied by 2. The assay was used following the manufacturer's recommendations. The principle of the assay is based on the difference in charge of phosphorylated (negatively charged) form versus the non-phoshorylated form of a fluorescent substrate. The two forms can be separated by gel electrophoresis and the negatively charged band excised and the fluorescence measured (exc. 440 nm and em. 590 nm) in 96 well plates on a Galaxy FluoStar plate reader. The PKI compounds were added to the assay mixture at 3 doses: 150, 300 and 600 μM and an extra 75 μM when the inhibition was too high. The assay is strictly biochemical and the doses of the compounds used, therefore, are generally not correlative with in vivo situations. The results are shown in Table 24.

TABLE 24
Effect of Compounds PKI 1-10 on PKC Isoforms (% Inhibition)

| | Dose/ | PKC Isoform | | | | | |
|---|---|---|---|---|---|---|---|
| Compound | μM | alpha | beta 1 | beta 2 | delta | epsilon | zeta |
| PKI 5 | 150 μM | 95 | 87 | 88 | 86 | 78 | 25 |
| | 300 μM | 94 | 90 | 79 | 91 | 81 | 71 |
| | 600 μM | 95 | 90 | 77 | 90 | 84 | 63 |
| PKI 6 | 150 μM | 76 | 89 | 87 | 86 | 59 | 24 |
| | 300 μM | 91 | 91 | 91 | 83 | 85 | 36 |
| | 600 μM | 92 | 92 | 91 | 85 | 85 | 54 |
| PKI 7 | 150 μM | 84 | 87 | 92 | 80 | 0 | 0 |
| | 300 μM | 94 | 87 | 95 | 83 | 67 | 15 |
| | 600 μM | 95 | 88 | 95 | 84 | 79 | 58 |
| PKI 4 | 150 μM | 80 | 72 | 83 | 90 | 55 | 60 |
| | 300 μM | 80 | 72 | 83 | 90 | 55 | 60 |
| | 600 μM | 80 | 82 | 82 | 95 | 77 | 58 |
| PKI 8 | 150 μM | 83 | 73 | 90 | 83 | 41 | 0 |
| | 300 μM | 87 | 81 | 94 | 86 | 77 | 28 |
| | 600 μM | 89 | 83 | 91 | 85 | 82 | 22 |
| PKI 1 | 150 μM | 78 | 71 | 78 | 56 | 54 | 15 |
| | 300 μM | 84 | 92 | 90 | 61 | 58 | 10 |
| | 600 μM | 87 | 93 | 92 | 88 | 83 | 20 |
| PKI 9 | 150 μM | 12 | 8 | −54* | −44* | 0 | 0 |
| | 300 μM | 70 | 88 | 58 | 9 | 0 | 0 |
| | 600 μM | 92 | 88 | 81 | 66 | 88 | 0 |
| PKI 2 | 150 μM | −24* | 18 | −59* | 59 | 58 | 0 |
| | 300 μM | 35 | 52 | 61 | 79 | 67 | 6 |
| | 600 μM | 83 | 70 | 74 | 84 | 84 | 20 |
| PKI 3 | 150 μM | 47 | 37 | 31 | 32 | 38 | 37 |
| | 300 μM | 51 | 76 | 83 | 41 | 53 | 40 |
| | 600 μM | 82 | 80 | 81 | 70 | 68 | 56 |
| PKI 10 | 150 μM | 6 | 7 | 26 | 84 | 62 | −27* |
| | 300 μM | 73 | 82 | 64 | 92 | 84 | 0 |
| | 600 μM | 75 | 81 | 76 | 89 | 85 | 17 |

*Negative values indicate that the compound had an activation effect on the enzyme.

As can be seen from Table 24, the PKI compounds show some specificity toward the PKC isoforms of the panel in that they are more active against the cPKCs (alpha, betaI/II), which share a similar structure for the catalytic site, and are less potent against the nPKCs (delta and epsilon) and aPKC (zeta), which are reported to have different catalytic site structures to that of the cPKCs.

Compounds PKI 1, 4, 5, 7 and 8 are very potent inhibitors of all the PKCs isoforms except PKC epsilon and zeta. At the higher concentration of 300 μM the inhibition of the PKCs alpha to delta is almost complete with all of these compounds. In contrast, in PKC epsilon, there is a dose response up to 600 μM, a dose that is not sufficient to achieve complete inhibition. A similar pattern is observed with PKC zeta, which is less sensitive than epsilon to the compounds. Replacement of the amino acids LRL in compound PKI 6 with RGR in compound PKI 1 appears to confer a higher specificity of the compound toward PKC alpha.

### Example 16

### Effect of Compounds PKI 1 to 10 on Cancer Cell Proliferation

The induction of cell death and the alteration of cell proliferation in 10 cell lines representative of different cancers were studied following individual incorporation of compounds PKI 1 to 10 into the cells via pinocytic influx. The compounds were used at concentrations of 5 mM and 10 mM. These concentrations do not, however, directly correlate with the amount of the compound actually received by the cells, as noted in Example 14. The cell lines employed were as follows: U-251 glioblastoma cell line; H-661 non-small cell lung cancer cell line; IMR-32 neuroblastoma cell line; LNCap and DU-145 prostate cancer cell lines; LS-180 colon cancer cell line; MCF-7 and MDA-MB-231 breast cancer cell lines; SKOV-3 ovarian cancer cell line and T-24 bladder cancer cell line. Breast cancer cell lines MCF-7 and MDA-MB-231 differ both in their expression of oestrogen receptors (MCF-7+) and aggressiveness. MDA-MB-231 is an oestrogen negative cell line expressing Her/Erb-2 and is representative of metastatic breast tumours. Similarly, of the two prostate cancer cell lines, LNCap is an androgen insensitive cell line and DU145 is an androgen positive cell line.

The methodology used was as follows. The starting cell suspension density was 1×10⁶ cells/ml. Each PKI compound was incorporated to each given cell line at 5 mM and 10 mM concentration in 10 μl by pinocytic endocytosis (Invitrogen/Molecular Probe) following supplier recommendations. 5000 cells were distributed in 96 well plates in appropriate media containing 10% FBS and allowed to grow for 24, 48 and 72 hrs. Following endocytosis, the cell suspension was plated as such without elimination of the dead cells. The size of the cell populations was further assessed as total DNA (a value that directly relate to the number of cells; see Example 14).

The experimental setting outlined above allowed the primary effect of each compound on cell death to be measured over the 24 h following incorporation of the PKI compound. The difference in population size of the control untreated cells and the treated cells at the time point 24 h thus measures the death toll. During the next 48 h the proliferation patterns reflect whether the compound alters growth and also indirectly informs on the stability of the compounds. Thus, the experimental set up permits the simultaneous estimation of apoptosis, proliferation index of the resistant or unloaded cells and persistence of the PKI compound in the cells or endogenous stability.

The percentage cell death at 24 h (short term) is shown in Tables 25 and 26.

TABLE 25
Percentage Cell Death at 24 h after Treatment with
Compounds PKI 1 to 10 at 5 mM

| | Cell-line | | | | | | | | | |
|---|---|---|---|---|---|---|---|---|---|---|
| | | | MDA- | | | | | | | |
| | | | MB- | | | | | | | |
| Compound | H661 | MCF-7 | 231 | SKOV-3 | LnCAP | Du145 | T24 | IMR32 | LS180 | U251 |
| PKI 5 | 17 | 15 | 22 | 5 | 1 | 20 | 14 | 11 | 42 | 6 |
| PKI 6 | 21 | 19 | 3 | 29 | 15 | 22 | 35 | 2 | 30 | 6 |
| NCI 7 | 7 | 29 | 27 | 14 | 5 | 5 | 17 | 7 | 28 | 17 |
| PKI 4 | 16 | 48 | 48 | 8 | 70 | 11 | 23 | 11 | 51 | 70 |
| PKI 8 | 11 | 59 | 13 | 12 | 24 | 28 | 5 | 9 | 21 | 4 |
| PKI 1 | 46 | 18 | 1 | 1 | 16 | 17 | 1 | 2 | 1 | 7 |
| PKI 9 | 8 | 31 | 34 | 21 | 15 | 36 | 12 | 5 | 24 | 11 |
| PKI 2 | 6 | 3 | 8 | 2 | 13 | 1 | 3 | 0 | 48 | 31 |
| PKI 3 | 1 | 25 | 1 | 5 | 3 | 29 | 1 | 19 | 9 | 7 |
| PKI 10 | 13 | 26 | 21 | 1 | 11 | 6 | 3 | 23 | 13 | 10 |

TABLE 26
Percentage Cell Death at 24 h after Treatment with
Compounds PKI 1 to 10 at 10 mM

| | Cell line | | | | | | | | | |
|---|---|---|---|---|---|---|---|---|---|---|
| | | | MDA- | | | | | | | |
| | | | MB- | | | | | | | |
| Compound | H661 | MCF-7 | 231 | SKOV-3 | LnCAP | Du145 | T24 | IMR32 | LS180 | U251 |
| PKI 5 | 22 | 36 | 36 | 43 | 4 | 25 | 28 | 27 | 56 | 16 |
| PKI 6 | 25 | 39 | 19 | 29 | 17 | 28 | 42 | 14 | 58 | 16 |
| PKI 7 | 17 | 36 | 29 | 36 | 16 | 11 | 17 | 13 | 55 | 21 |
| PKI 4 | 20 | 60 | 52 | 22 | 76 | 13 | 29 | 24 | 53 | 76 |
| PKI 8 | 18 | 79 | 19 | 31 | 30 | 52 | 11 | 11 | 28 | 44 |
| PKI 1 | 12 | 21 | 5 | 9 | 19 | 14 | 4 | 7 | 2 | 16 |
| PKI 9 | 8 | 56 | 40 | 22 | 23 | 43 | 17 | 17 | 44 | 29 |
| PKI 2 | 34 | 29 | 13 | 14 | 11 | 8 | 3 | 0 | 52 | 32 |
| PKI 3 | 19 | 15 | 21 | 16 | 22 | 32 | 22 | 21 | 12 | 14 |
| PKI 10 | 16 | 29 | 33 | 5 | 23 | 40 | 28 | 30 | 18 | 21 |

All compounds were able to inhibit at least two of the tested cancer cell line by 15% or more at a concentration of 5 mM. While there appears to be a certain amount of specificity of the compounds toward various cell lines, interestingly, the colon cancer cell line LS-180 was very sensitive to cell death induced by the majority of compounds and notably by compounds PKI 5, 6, 7, 4, 9 and 2. Both compounds PKI 4 and PKI 2 exerted a powerful short-term activity on this colon cancer cell line followed by moderate re-growth. The most striking effect of the compounds is on cell death over the first 24 h following incorporation of the compounds. In addition, the stability of the compounds is at least of 72 h based on the general maintenance of the growth at steady state or decreasing proliferation.

### Example 17

### Effect of Compounds PKI 1 to 10 on Apoptosis

Short-term cell death resulting from the internalisation of compounds PKI 1 to 10 is illustrated in Tables 25 and 26 above. DNA laddering could not, however, be clearly observed as DNA extracts showed up as smears on the gels. Accordingly, the effect of a representative compound, compound PKI 3, was analysed as a dose response in two cancer cell lines in which the compound did not show any short term cell death, MDA-MB-231 breast cancer cell line and NCI H-661 non-small cell lung cancer cell line, using Hoechst reagent to stain the nuclei. Cells were treated with compound PKI 3 at concentrations of 50, 250 and 500 Hoechst staining was performed as follows. Cells were washed 1× with phosphate-buffered saline (PBS) containing Ca²⁺ and Mg²⁺. Cells were then fixed with 1% paraformaldehyde solution prepared in PBS containing Ca²⁺ and Mg²⁺ for 20 min at room temperature and washed 3× with PBS containing Ca²⁺ and Mg²⁺. Cells were stained with 5 μg/mL Hoechst 33258 in PBS for 20 minutes to detect chromatin packing, a marker of apoptosis. Finally the cells were washed 3× with PBS. Images were collected on a Nikon Olympus Microscope using IMT2-DMV filter at an Excitation 405 ηm and emission greater than 455 ηm and analyzed on Image Pro Software.

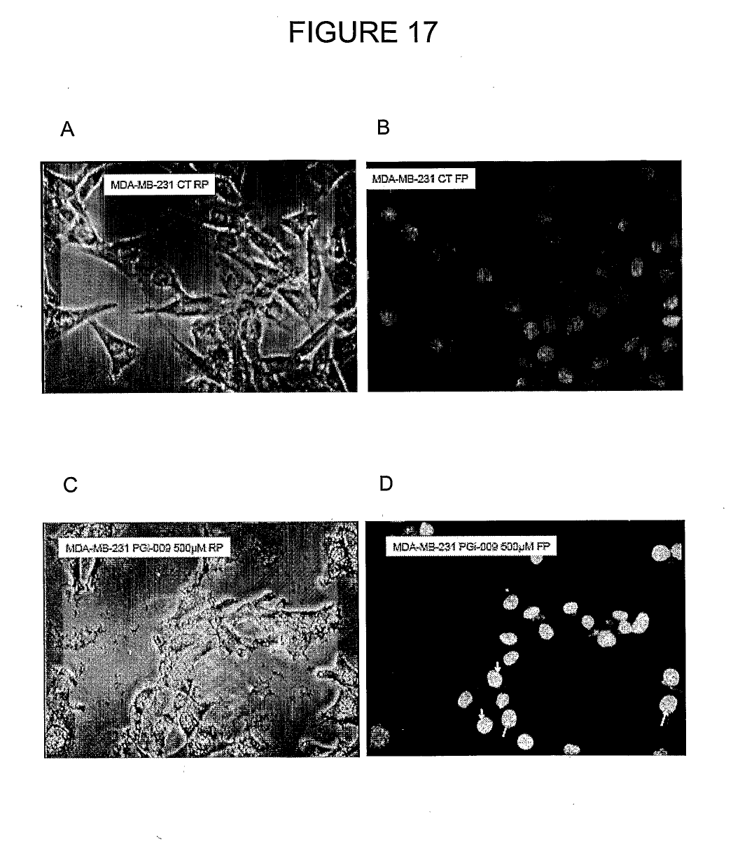

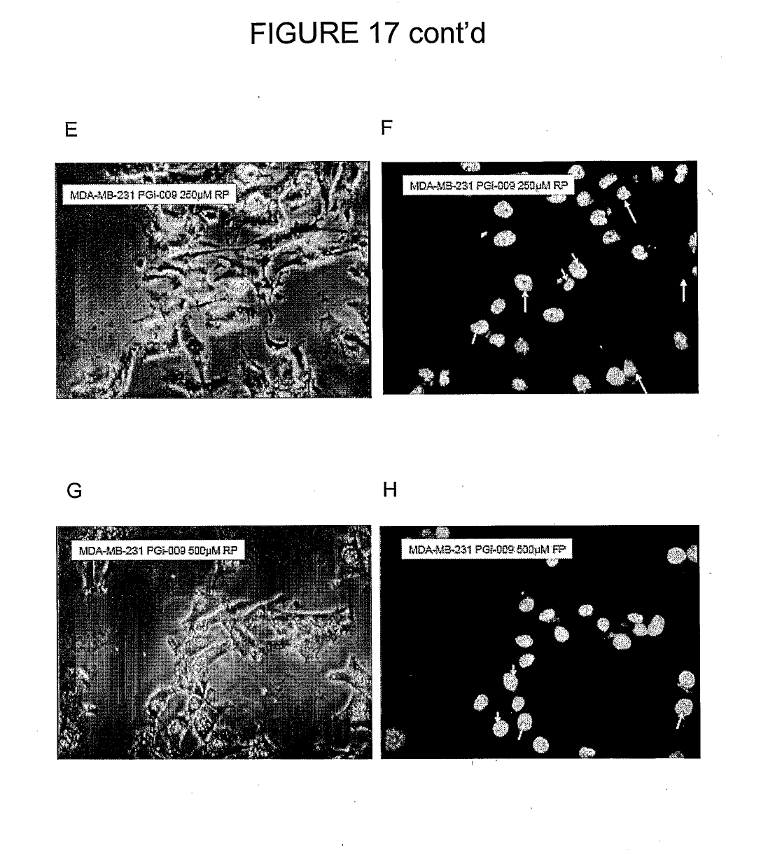

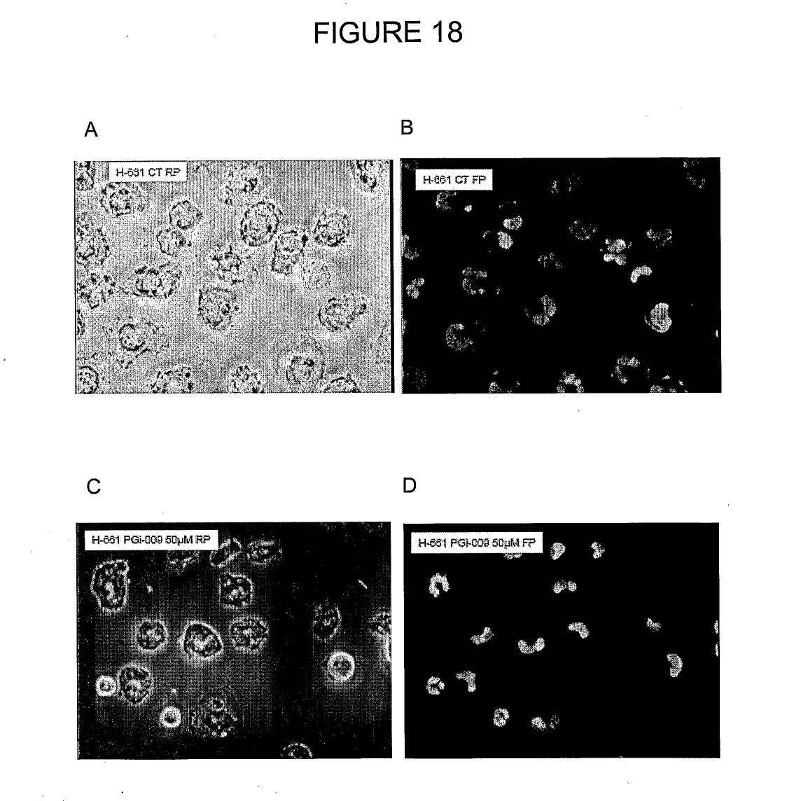

The results are shown in FIG. 17 (MDA-MB-231) and FIG. 18 (H-661). All images are 20× in magnification except for FIG. 18, panel E, which is 10× magnification. The results are shown as matching images: The left side images are reverse phase and the right side images show the nuclei stained with Hoechst reagent. The two cell lines show a clear dose response with increasing cytopathy as shown by extensive vacuolization of the cytoplasm. Cell death is shown as birefringent cells especially in the 10× magnification reverse phase image (FIG. 18, panel E). Although no clear DNA laddering was obtained, chromatin packing and eccentric chromatin location suggest that apoptotic signals were triggered by compound PKI 3.

### Example 18

### Effect of Compounds PKI 1 to 10 on Cell Migration/Invasion

MDA-MB-231, an invasive breast cancer cell line (Epidermal growth factor positive), was used for measuring the migration inhibition potential of compounds using standard protocols based on migration of cells through a Matrigel matrix (see Example 29). All treatments were made in triplicate. The results are shown in Table 27. Data are expressed as the percent invasion through the Matrigel matrix and membrane related to the migration through the control membrane. % invasion and % invasion inhibition are calculated as follows:

$$\% Invasion = \frac{mean \# of cells invading through matrigel insert membrane}{mean \# of cells migrating through control insert membrane} \times 100$$

$$\% Invasion inhibition = \% Invasion in Control cells - \% invasion compound \text{-} treated cells$$

TABLE 27
Inhibition of Invasion of MDA-MB-231
cells by PKI Compounds

| | PKI Compound | | | | | | |
|---|---|---|---|---|---|---|---|
| | 1 | 2 | 3 | 4 | 6 | 8 | 9 |
| % Inhibition of Invasion | 5.8 | 2.0 | 29.8 | 29.1 | 8.2 | 1.7 | 6.5 |

Compounds PKI 5, PKI 7 and PKI 10 were tested for their motility by a method described by Zhang W. et al. ((2003) J. Neurosurgery, 99(6):1039-46). In brief, MDA-MB-231 cells were plated in the centre of round petri dishes 10 cm in diameter at a density of 2×10⁴ cells in 200 μl of RPMI+10% PBS. Prior to plating the control and treated cells, 4 circles were drawn on the outer side of the base of the petri dishes. After 6 h incubation at 37° C. in a humidified 5% CO₂ atmosphere, the medium was removed and discarded and a circular zone of adherent cells in the centre of the Petri dish was formed. These cells were washed with medium without serum and were supplemented with fresh medium containing serum. The culture was incubated at 37° C. for a further 6 days.

To determine cell motility, the number of cells at a predetermined distance from the perimeter of the central zone was counted daily in triplicate and % motility was calculated relative to control cells, which were considered as 100% motile.

After 5 days, the inhibition of motility was 3.5%, 3.0% and 5.0% for compounds PKI 10, PKI 7, and PKI 5, respectively. No inhibition was observed at day 3 or 4.

### Example 19

### Preparation of Exemplary Targeted Inhibitory Molecule (TIM) Comprising a PRE Conjugated to a PKI Compound

The following TIMs comprising a PRE conjugated to a PKI compound were synthesized. A representative synthesis protocol is provided below for compound TIM 10.

Compound TIM 9 was synthesized as two separate chains and coupled together after synthesis using standard protocols and the reagents (NH₄)₂CO₃/AcOH/DMSO.

Compound TIM 11 was synthesized by coupling the cell permeability enhancing peptide RRRQRRKKR to the N-terminus of compound TIM 9, lower peptide chain (as shown in Table 28), using the coupling technique recommended by the manufacturer (employing HBTU/HOBt/DIPEA). The N-terminus of the cell permeability enhancing peptide was then acetylated by standard techniques and the two peptide chains of the compound subsequently coupled together as described above for compound TIM 9.

TABLE 28
Exemplary TIM compounds comprising a PRE conjugated to a PKI
compound

| Compound | |
|---|---|
| (PRE + PKI | |
| components) | Structure |
| TIM 6 (PRE 11 + PKI 3) | 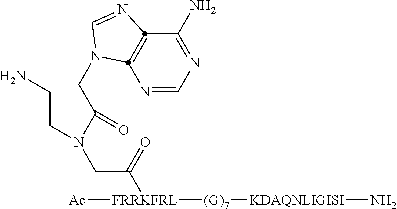 |
| TIM 7 (PRE 4 + PKI 10) | 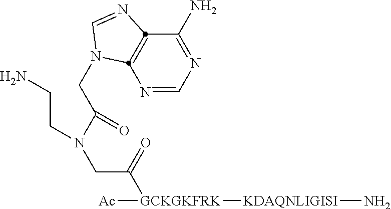 |
| TIM 8 (PRE 4 + PKI 3) | 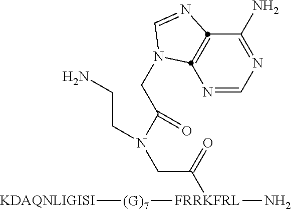 |
| TIM 9 (PRE 4 + PKI 1) | 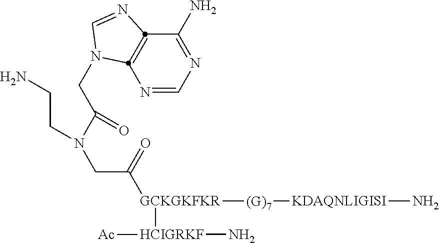 |
| TIM 10 (PRE 4 + PKI 3) | 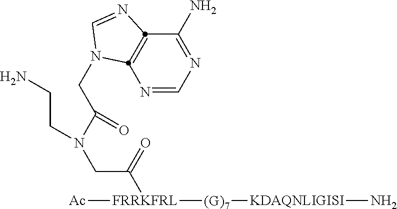 |
| TIM 11 (PRE 4 + PKI 1) | 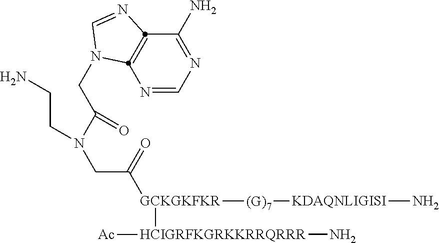 |
| TIM 12 (PRE 10 + PKI 3) | 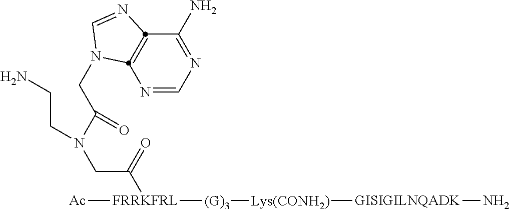 |
| TIM 13 (PRE 4 + PKI 3) | 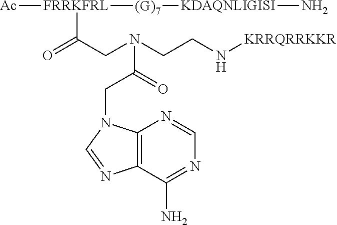 |
| TIM 14 (PRE 4 + PKI 3) | 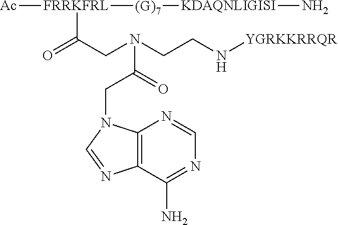 |
| TIM 15 (PRE 4 + PKI 3) | 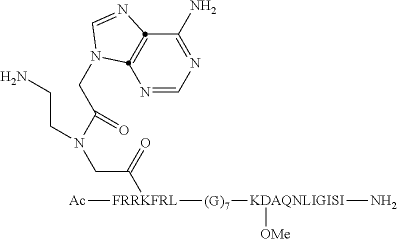 |
| TIM 16 (PRE 4 + PKI 3) | 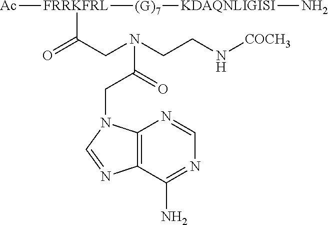 |
| TIM 17 | FRRKFRL—(G)₇—KDAQNLIGISI |
| (PRE 4 and SEQ ID | |
| NO: 37) | |
| TIM 18 | Ac—FRRCFRL—(G)₇—AKGIQEVKGGDAQNLIGISI—NH₂ |
| (PRE 3 and PKI 4) | |
| TIM 19 (PRE 3 and PKI 9) | 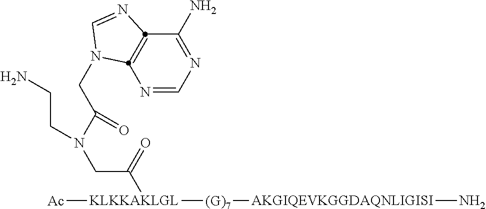 |
| TIM 20 | Ac—FRRCFRL—(G)₇—RRKKGGKDFVVKR—NH₂ |
| (PRE 1 and PKI 4) | |
| TIM 21 | Ac—FRRCFRL—(G)₇—KDAQNLIGISI—NH₂ |
| (PRE 4 and PKI 4) | |
| TIM 22 (PRE 4 and PKI 5) | 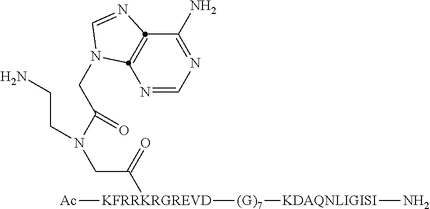 |

The general synthesis procedure for compound TIM 10 was as follows. Firstly, the peptide chain was synthesized on a Applied Biosystems Pioneer Peptide Synthesizer following the protocol provided by the manufacturer and employing Lys in which the side chain is protected with the amine protecting group ivDde.

After the peptide chain was synthesized, the ivDde protecting group was removed by washing the resin with DMF, isopropanol and dichloromethane, allowing the resin to dry for 20 mins, then washing for 30 mins with 2% Hydrazine (in DMF). The resin was then washed again with DMF, isopropanol and dichloromethane and allowed to dry.

The adenine PNA(Bhoc) was coupled to the side chain of the Lys residue in the peptide chain by shaking the resin in DMF solvent with 2 eq activator HBTU/HOBt, 2 eq DIPEA and 2 eq PNA(Bhoc). After 12 hrs, the resin was washed and then submitted to a de-protection step to remove the Fmoc from the PNA by shaking the resin with 20% piperidine/DMF for 6 hrs. Finally, the peptide was cleaved from the resin using standard protocols. After filtering, the peptide was dissolved in H₂O (0.1% TFA) and purified by column chromatography.

### Example 20

### In Vitro Inhibition of Cancer Cell Proliferation with Compound TIM 9

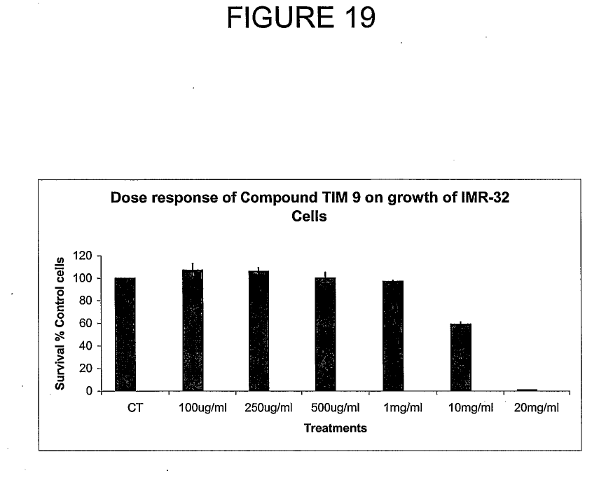

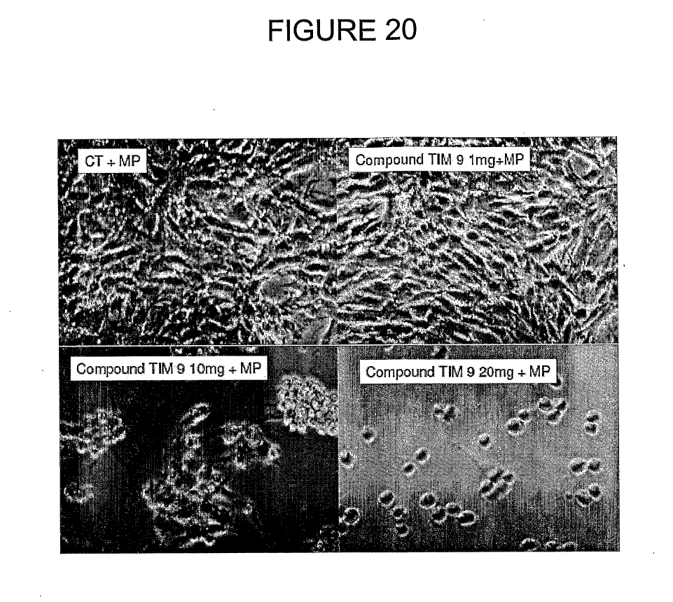

The ability of the compound TIM 9 to inhibit cancer cell proliferation was tested in vitro using the human neuroblastoma cell line IMR-32 and following the general protocol described in Example 14. The results after 48 hours of treatment are shown in Table 29 and FIG. 19. FIG. 20 shows the morphology of IMR-32 cells treated with compound TIM 9. The dose response to compound TIM 9 (B, C & D) in comparison to control (A) shows that at high concentrations of compound TIM 9, the cells round up and die.

TABLE 29
Inhibition of Proliferation of IMR-32 Cells by Increasing Doses of Compound 9

| | | Compound TIM 9 | | | | | |
|---|---|---|---|---|---|---|---|
| | | 100 μg | 250 μg | 500 μg | 1.00 mg | 10.0 mg | 20.0 mg |
| | Control | (0.036 mM) | (0.09 mM) | (0.18 mM) | (0.36 mM) | (3.6 mM) | (7.2 mM) |
| % | 100 | 107 | 106 | 100 | 97 | 59 | 0.9 |
| Survival | | | | | | | |
| % Growth | 0 | 0 | 0 | 0 | 3 | 41 | 99.1 |
| Inhibition | | | | | | | |
| SD | 0 | 6 | 3 | 5 | 1 | 2 | 0 |

### Example 21

### In Vitro Inhibition of Cancer Cell Proliferation with Compound TIM 11

In order to improve the amount of the TIM that penetrates the cells, compound TIM 9 was modified by co-synthesis with the cell permeability enhancing peptide RRRQRRKKR as described (see Example 19 and Table 28) to provide TIM 11. The peptide likely does not change the structure of compound TIM 11 as it detaches from the compound following internalization (Bárány-Wallje E et al. (2005) Biophys. J. BioFast, Jul. 22, 2005).

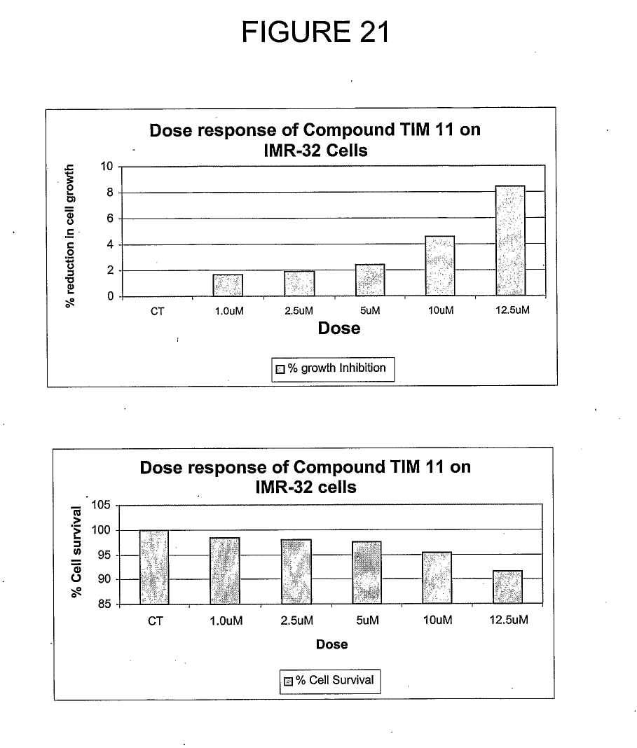

The ability of the compound TIM 11 to inhibit IMR-32 cancer cell proliferation was tested in vitro following the general protocol described in Example 14. The results after 24, 48 and 72 hours of treatment are shown in Table 30 and FIG. 21 (24 hours). Note that the doses of compound TIM 11 are approximate as possibly not all the compound has entered the cells.

The treated IMR32 cells also underwent morphological changes after 24 hrs of treatment with compound TIM 11 at various dosages. At a dose as low as 2.5 μM, cytoplasm enlargement and stress fibres appear, and at a dose of 5 μM dying cells can be seen. At doses of 10 to 25 μM stress fibres and cytoplasmic enlargement can be seen together with signs of cytopathy as the dosage increases. At a dose of 50 μM the ratio of cytoplasm/nucleus becomes dramatically reduced. Apoptotic bodies can be observed in almost each cell. At a dose of 100 μM the cells have differentiated and exhibit cytopathic vacuolization and at a dose of 250 μM all cells have died.

TABLE 30
Inhibition of Proliferation of IMR-32 Cells by
Increasing Doses of Compound TIM 11

| | 24 Hours | | 48 Hours | | 72 Hours | |
|---|---|---|---|---|---|---|
| | % Cell | % Growth | % Cell | % Growth | % Cell | % Growth |
| Compound | Survival | Inhibition | Survival | Inhibition | Survival | Inhibition |
| Control | 100 | 0 | 100 | 0 | 100 | 0 |
| Compound 11 | | | | | | |
| 1.0 μM | 98 | 2 | 92 | 8 | 94 | 6 |
| 2.5 μM | 98 | 2 | 90 | 10 | 93 | 7 |
| 5.0 μM | 98 | 2 | 91 | 9 | 92 | 8 |
| 10.0 μM | 95 | 5 | 92 | 8 | 95 | 5 |
| 12.5 μM | 92 | 8 | 93 | 7 | 93 | 7 |
| 25.0 μM | 80 | 20 | 90 | 10 | 93 | 7 |
| 50.0 μM | 73 | 27 | 80 | 20 | 90 | 10 |
| 100.0 μM | 66 | 34 | 64 | 36 | 59 | 41 |
| 250.0 μM | 30 | 70 | 35 | 65 | 26 | 74 |

### Example 22

### Inhibition of PKC Activity by Compound TIM 9 in Human Neuroblastoma Cells (IMR-32)

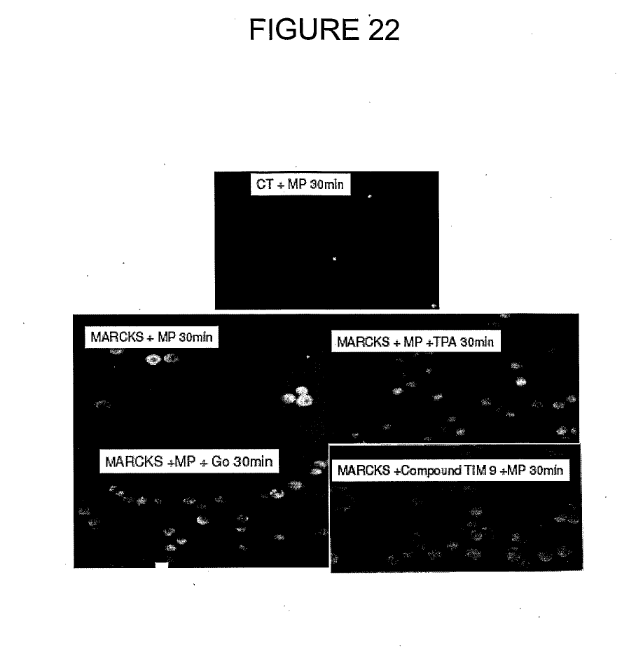

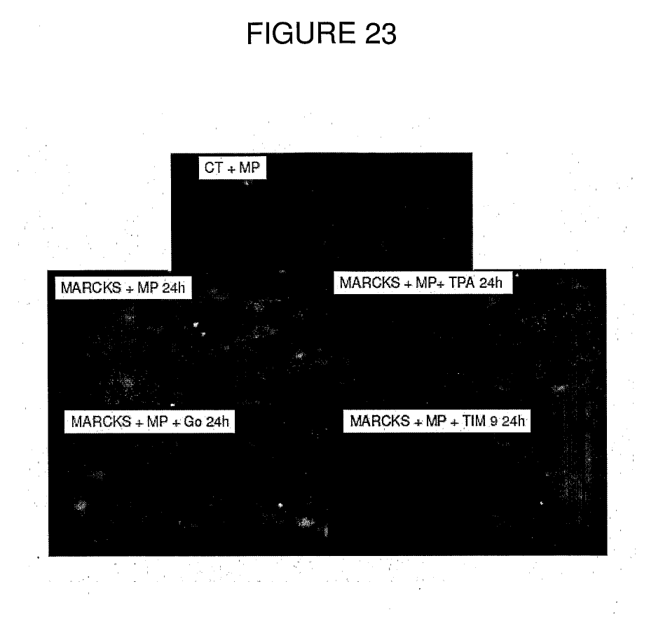

The ability of compound TIM 9 to inhibit phosphorylation of a natural substrate of PKC enzymes, the MARCKS peptide (154-165) (Signal Transductions Products, Catalog #S-1301) was investigated in IMR-32 cells. The MARCKS peptide was incorporated into the cells using pinocytic endocytosis (MP) and detected following conventional fixation procedure and immuno-cytochemical detection with a rabbit anti-phosphorylated MARCKS specific antibody (Proteintech Group Inc. Catalog #10018-3-AP). The results are shown in FIGS. 22 and 23. FIG. 22 shows control cells without injected MARCKS (top panel), after MARCKS incorporation (upper left panel), after treatment by TPA for 30 min (upper right panel), after treatment with a known inhibitor of classical PKCs (Go6976) (lower left panel) and after treatment with TPA and compound TIM 9 (lower right panel). As can be seen from FIG. 22, control cells show limited expression of MARCKS. Following TPA treatment for 30 min, there is an increase in the peptide phosphorylation indicative of the presence of active PKCs. The inactivation of cPKCs by Go6976 is still very low after 30 min exposure, whereas after 30 min treatment with compound TIM 9, inactivation of cPKCs is already noticeable.

FIG. 23 presents the results obtained after 24 h following the same treatments as those detailed in FIG. 22. Control cells show limited expression of MARCKS. Following TPA treatment for 24 hours, there is a decrease in the peptide phosphorylation indicative of the known effect of TPA treatment on PKCs (activation upon short term exposure, inactivation after longer term exposure) (top right panel). The inactivation of cPKCs by Go6976 is obvious as shown by the decrease in fluorescence emission estimation of the phospho-MARCKS endogenous levels (lower left panel). By comparison, compound TIM 11 demonstrates a more drastic effect (lower right panel).

### Example 23

### Inhibition of Non-Small Cell Lung Cancer Cell Proliferation by Compound TIM 10

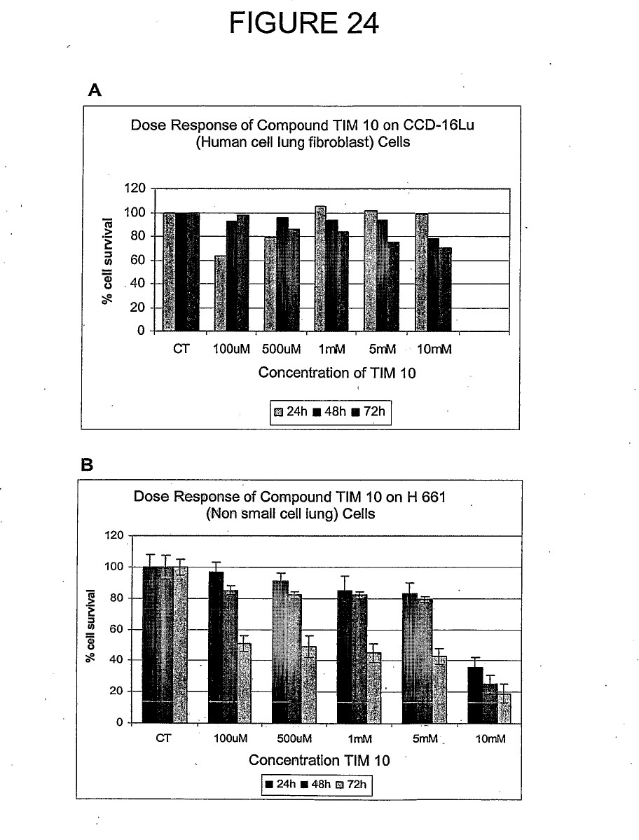

The ability of the compound TIM 10 to inhibit cancer cell proliferation was tested in vitro using the human non-small cell lung cancer cell line H661 and following the general protocol described in Example 14. The effect of compound TIM 10 on CCD-16Lu cells (normal immortalized human lung fibroblasts) was also assessed by the same protocol. The results after 24, 48 and 72 hours of treatment are shown in Table 31 (H661 cell line) and FIG. 24 (A: CCD-16LU cell line, and B: H661 cell line). The change in dose effect at 48 and 72 hrs in normal cells (see FIG. 24A) for lower doses of compound TIM 10 was interpreted as being due to the probable intracellular degradation of compound TIM 10 which would reduce the level of compound TIM 10 to doses that no longer affect growth. In H661 cells, however, at doses of 1 mM compound TIM 10 was not degraded and its sub localization was not altered as assessed by western blot and immunocytochemistry imaging.

TABLE 31
Inhibition of Proliferation of Human NSCLC (H661) Cells by
Increasing Doses of Compound TIM 10

| | % Cell Survival (±S.D.) | | |
|---|---|---|---|
| Compound | 24 Hours | 48 Hours | 72 Hours |
| Control | 100 (8) | 100 (7.5) | 100 (5) |
| TIM 10 | | | |
| 100 μM | 97 (6) | 85 (3) | 51 (5) |
| 500 μM | 91 (5) | 82 (2) | 49 (7) |
| 1 mM | 85 (9) | 82 (2) | 45 (6) |
| 5 mM | 83 (7) | 79 (2) | 43 (5) |
| 10 mM | 36 (6) | 25 (6) | 19 (6) |

Of interest in the results shown in FIG. 24 is the fact that normal CCD-16LU lung cells are less affected by compound TIM 10 than the H661 cancer cells from the same organ. The H661 cells were extremely sensitive to compound TIM 10 and inhibition in cell survival was dose and time dependent. The cells were shown to be blocked in the G2 phase of the cell cycle (see Example 32).

### Example 24

### Inhibition of Neuroblastoma Cell Proliferation by Compound TIM 10

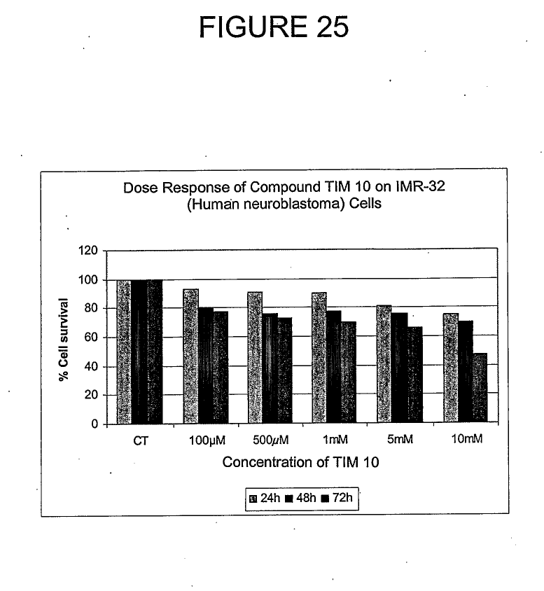

The ability of the compound TIM 10 to inhibit cancer cell proliferation was tested in vitro using the human neuroblastoma IMR-32 cell line and following the general protocol described in Example 14. The data are presented in Table 32 and FIG. 25. This aggressive human cancer cell line showed 25% growth inhibition 24 h after treatment with compound TIM 10 at 10 mM. The highest growth inhibition was obtained after 72 h, which is in agreement with previous data obtained using an antisense oligonucleotide to PKC-α. These data also suggest that in neuroblastoma cells the effect of compound TIM 10 lasts for at least 72 h.

TABLE 32
Inhibition of Proliferation of Human Neuroblastoma
IMR-32 Cells by Increasing Doses of Compound TIM 10

| | % Cell Survival | | |
|---|---|---|---|
| Compound | 24 Hours | 48 Hours | 72 Hours |
| Control | 100 | 100 | 100 |
| Compound 10 | | | |
| 100 μM | 93 | 80 | 77 |
| 500 μM | 91 | 76 | 73 |
| 1 mM | 90 | 77 | 70 |
| 5 mM | 81 | 76 | 66 |
| 10 mM | 75 | 70 | 47 |

### Example 25

### Restoration of Gap Junction Function in Cancer Cells by Compound TIM 10

The ability of compound TIM 10 to restore gap junction function was assessed in human neuroblastoma (IMR-32) cells. IMR-32 cells were grown on a cover slip as a monolayer. After 24 hrs, cells were first treated with forskolin, which increases the intracellular level of connexin, and were then injected with compound 10 (50 μM) via pinocytic influx. Control, c-AMP and compound 10-treated monolayers were incubated at 37° C. and then subjected to scrape loading with Lucifer yellow. The cell permeant analogue of cAMP (8-bromo-cAMP) was used in these experiments. Dye movement through the gap junctions could clearly be seen in fluorescence photomicrographs after 3 h and 24 h.

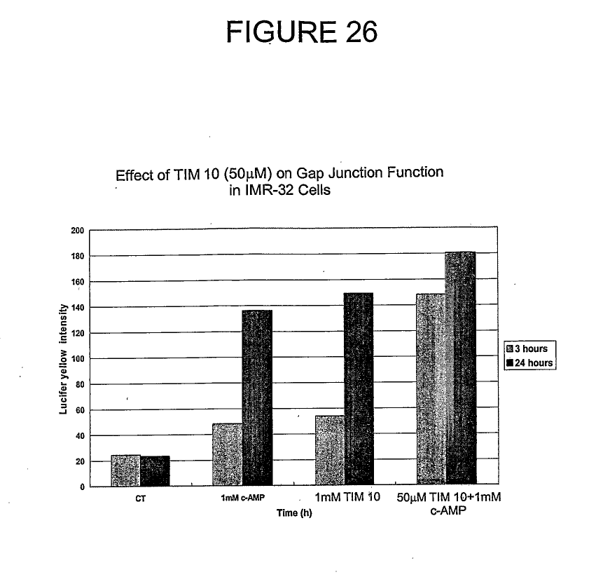

Quantitative assessment of gap junction function is presented in FIG. 26 and shows the intensity (measured by image PRO plus 4.5 software) of Lucifer yellow dye movement through gap junctions in IMR-32 cells 3 h and 24 h after treatment with compound TIM 10 (50 μM). FIG. 26 clearly shows that compound TIM 10 was able to restore gap junction function in these cells.

### Example 26

### Effect of Compound TIM 10 on Survival of Doxorubicin-Resistant Human Colon Cancer Cells

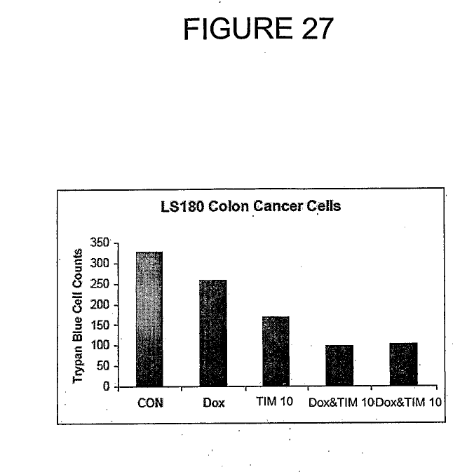

Highly tumourigenic human colon cancer cells (LS180; ATCC # CL-187) which constitutively express multi-drug resistance (MDR) were used for this experiment. LS180 cells were seeded in 6 well plates at a density of 2×10⁵. After a 24 hr recovery cells were treated with 50 ng/ml of doxorubicin, 2.5 μM compound TIM 10, or a combination of doxorubicin and compound TIM 10 (2.5 μM and 5 μM) for 72 hrs. Note that compound TIM 10 was added to the cell culture medium; no protocol was used to force the compound into the cell. The cells were then trypsinized and counted using the standard trypan blue dye exclusion assay. The results are shown in FIG. 27 (bars represent the average of two experiments) and demonstrate that a large proportion of the cells survived treatment with doxorubicin, indicating that the cells are drug resistant, but compound TIM 10 alone was capable of causing cell death. The combination of compound TIM 10 and doxorubicin was highly effective at causing cell death in LS180 cells previously resistant to doxorubicin.

### Example 27

### Effect of Compound 10 on Drug Resistance of Human Colon Cancer Cells

The determination of whether multi-drug resistance (MDR) is expressed and functional in a cancer cell line is generally made by measuring the fluorescence leakage of calcein AM or rhodamine 123 from the cells. The efflux of these dyes simulates the way in which chemotherapeutic agents would be pumped out of the cell. Cancer cells loaded with calcein AM will efflux the dye if the cells express either Pgp or MRP-1 (two families of MDR proteins), whereas rhodamine 123 will efflux only from cells that express Pgp. The ability of compound TIM 10 to affect the efflux of both calcein AM and rhodamine 123 from human colon cancer cells (LS180) was determined as described below.

LS180 cells were seeded in petri dishes at 4×10⁵ and left to recover overnight. The following day doxorubicin (50 ng/ml) and/or compound 10 (2.5, 5 or 10 μM) was added and the cells incubated for the required time period. Note that compound 10 was added to the cell culture medium; no protocol was used to force the compound into the cell. At the end of 72 hrs, cells were loaded with either 2.5 mM calcein AM in DMSO or 200 ng/ml of Rhodamine 123 in DMSO and were incubated in the dye for 30 min (calcein) or 1 hr (RHO123). After incubation, the dye was removed and replaced by fresh medium and the cells were left at 37° C. for 90 minutes to efflux the dye. Cells were then trypsinized and resuspended in 1 ml of medium in preparation for flow cytometry.

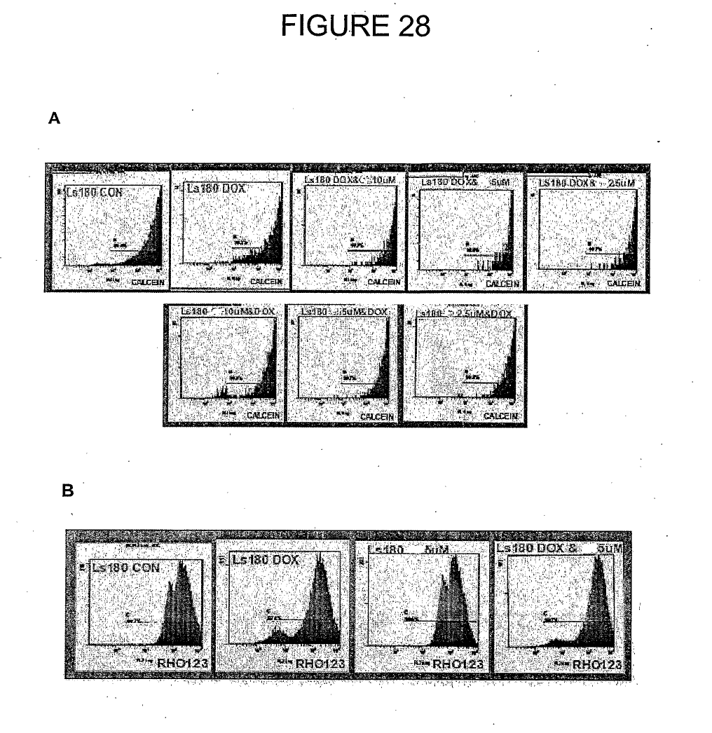

The results are shown in FIG. 28. (A) compound TIM 10 (10, 5 and 2.5 μM) was effective in decreasing MDR-mediated calcein efflux. The top row of this figure represents addition of compound TIM 10 in the last 24 hrs of the 72 hr treatment, and the lower row represents addition of compound 10 for the full 72 hr treatment. In untreated LS180 colon cancer cells a constitutive level of MDR is present as indicated by the fluorescence (representing dye efflux) moving towards the Y (left) axis. In cells treated with 50 ng/ml of doxorubicin for 72 hrs the dye efflux increases due to the increase in the level of functional MDR. Addition of compound TIM 10 caused a substantial decrease in calcein dye efflux as indicated by the retreat of the fluorescence towards the right axis. (B) compound TIM 10 (5 μM) was effective in decreasing MDR-mediated rhodamine 123 efflux. As was the case for calcein efflux, the presence of compound TIM 10 caused a substantial decrease in rhodamine 123 dye efflux as indicated by the retreat of the fluorescence towards the right axis.

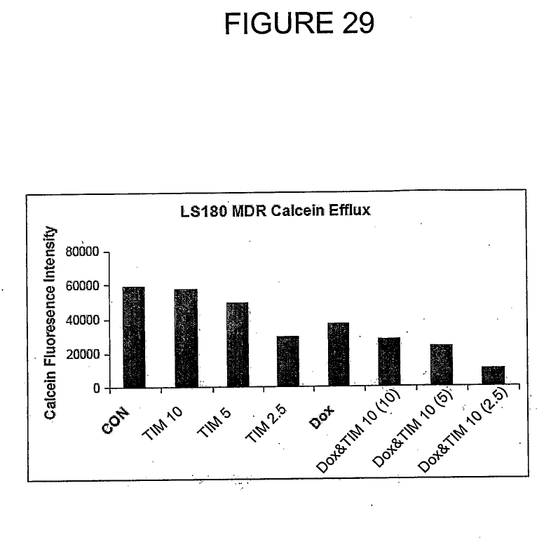

Calcein fluorescence was also quantitated by seeding the cells in 12 well plates and determining the relative intensity across wells using a Galaxy plate reader. This method produced identical results to the flow cytometry data as shown in FIG. 29 for compound TIM 10 at 2.5, 5 and 10 μM.

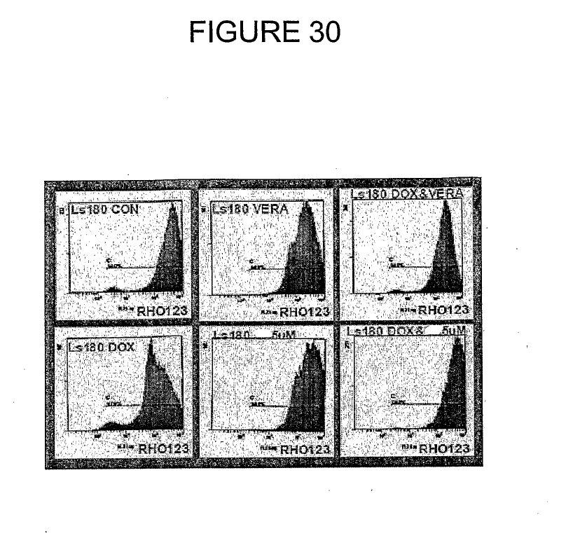

The effect of compound TIM 10 on rhodamine 123 efflux was also compared to the effect of the known and validated MDR inhibitor, Verapamil. Verapamil is highly effective at decreasing MDR in vitro but is considered too toxic for in vivo and clinical use. As shown in FIG. 30, compound TIM 10 at 5 μM was more effective in inhibiting Pgp-mediated MDR in LS180 cells than 5 μg/ml Verapamil. Expression of Pgp protein in LS180 cell stocks cultured in doxorubicin was confirmed by immunohistochemical studies.

### Example 28

### Effect of Compound TIM 10 on Levels of PKC-α and Connexin 43 in Human Colon Cancer Cells

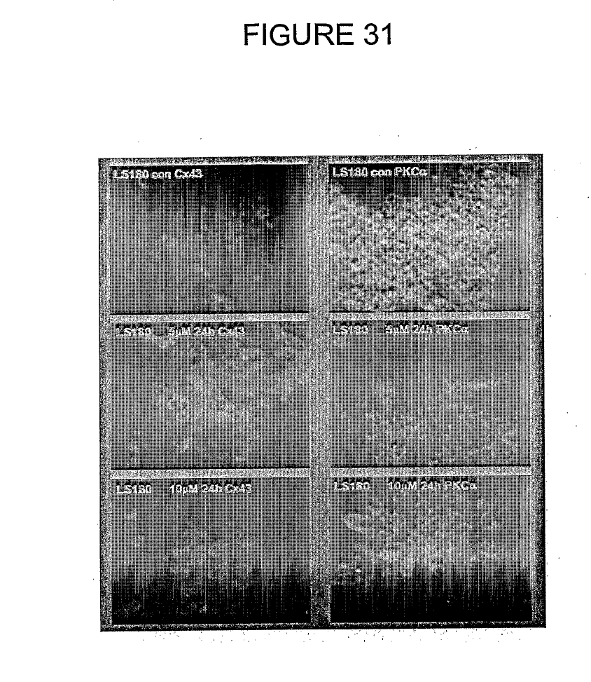

Human colon cancer cells (LS180) were incubated with compound TIM 10 (5 or 10 μM) for 24 hrs and the levels of connexin 43 (Cx43) and PKC-α proteins were evaluated by immunocytochemistry. As shown in FIG. 31, left hand column, the level of Cx43 protein was increased in cells treated with compound 10. Levels of PKC-α protein were decreased in cells treated with compound TIM 10 (FIG. 31, right hand column). This is consistent with previous observations that the gating of the gap junction channels is altered by PKC-α with consecutive loss of gap junction function, suggesting that Cx43 expression may be suppressed by PKC-α.

### Example 29

### Effect of Compound TIM 10 on Cancer Cell Migration/Invasion

Experiments were conducted to investigate the effect of compound TIM 10 (5 mM dose) on migration of human breast cancer cells (MDA MB231) with and without EGF (10 ng/ml) as a chemo-attractant. A standard a two-chamber culture system (BD BIOcoat™ Matrigel™ Invasion Chamber; BD Biosciences Clont, Discovery labware, Immunocytometry system Pharmingen) was employed for these experiments. Control chambers used in the following experiment are similar to the invasion chambers except that no Matrigel matrix is present, thus allowing cells to freely reach the lower membrane surface through the membrane pores.

Basal invasion activity was measured against FBS as an attractant, while invasion was measured against the chemoattractant EGF.

To measure basal invasion activity, cells were seeded on the upper chamber of the Invasion or Control Chamber in serum free medium and 10% FBS was supplied in the medium below the membrane. Migration of cells was assessed after 48 hrs. The culture was stained with Hoescht reagent to stain the cell nuclei. The results are shown in Table 33 and are expressed as the percent invasion through the Matrigel Matrix and membrane relative to the migration through the Control membrane (no Matrigel), i.e.

$$\% Invasion = \frac{Mean \# of cells invading through Matrigel insert membrane}{Mean \# of cells migrating through control insert membrane} \times 100$$

TABLE 33
Inhibition of Basal Invasion Activity of MDA MB231
Breast Cancer Cells by Compound TIM 10

| | | % Inhibition of |
|---|---|---|
| | % Invasion | Invasion |
| Untreated | 40 | 0 |
| Cells | | |
| TIM 10 | 4.5 | 35.5 |
| (5 mM) | | |

The ability of compound TIM 10 to inhibit invasion/migration of MDA MB231 cells in the presence of the chemoattractant EGF was measured as described above using EGF 10 ng/mL as the chemoattractant. After 48 h cells showed cytopathy (vaculated cytoplasm) with elongated processes and chromatin condensation was very clear in almost all the cells that had migrated to the lower side of the membrane. The results indicate that compound TIM 10 has a strong inhibitory impact on the migration MDA MB-231 even in the presence of 10 ng/ml EGF as chemoattractant.

Compound TIM 10 was also observed to exert a double effect on the MDA MB231 cells, firstly in blocking cell migration and secondly in killing the cells. The latter effect prevented calculation of the % of migration of inhibition.

Note that 25% inhibition in cell growth was observed after 24 h of treatment with compound TIM 10 (5 mM) in the presence of 10 ng/ml EGF.

### Example 30

### Specificity of Compound TIM 10

The effect of compound TIM 10 on the levels of protein for various PKC isoforms was assessed as generally described below in the following human cancer cell lines: MDA MB-231 breast adenocarcinoma cells (ATCC HTB-26); T24 bladder transitional cell carcinoma (ATCC HTB-4); SKOV-3 ovary adenocarcinoma (ATCC HTB-77); MCF-7 breast adenocarcinoma (ATCC HTB-22); Capan-2 pancreatic adenocarcinoma (ATCC HTB-80); NCI-H661 NSCL large cells (ATCC HTB-183); Calu-6 probable lung anaplastic carcinoma (ATCC HTB-56) and Calu-3 lung adenocarcinoma (ATCC HTB-55).

Cells representative of the different cancer types were cultured according to ATCC instructions. At subconfluence, the cells were trypsinized and compound 10 (5 mM) was incorporated into the cells by pinocytic influx endocytosis according to the manufacturer's instructions (Molecular Probe). Matching controls were treated accordingly except that vehicle was incorporated instead of compound 10. Following pinocytic influx, the cells were seeded and allowed to grow in fresh medium for 24 hrs. The cell cultures were further extracted using RIPA lysis buffer according to standard protocol. The protein content of each lysate was measured using Bradford protein estimation procedure (BioRad) and normalized. Following addition of sample buffer, the proteins from 15 μl of each extract were separated by electrophoresis, electro-transferred to nitrocellulose membranes and the various isoforms of PKC were detected using appropriate antibodies (Santa Cruz Biotechnology, Inc., CA). The relative intensity of bands of the expected molecular weight was estimated after scanning and the % decrease in the band intensity relative to control was calculated. The results are shown in Table 34.

TABLE 34
Effect of Compound TIM 10 on Levels of Various
PKC Isoforms in Cancer Cell Lines

| | % decrease | | | | |
|---|---|---|---|---|---|
| Cell line | PKC-α | PKC-δ | PKC-βI | PKC-ε | PKC-ζ |
| MDA-MB231 | 36.6 | 36.6 | 0.0 | 0.0 | 0.0 |
| T24 | 42.5 | 62.2 | — | — | 0.0 |
| SK-OV-3 | 63.4 | 0.0 | — | — | — |
| MCF-7 | 12.0 | 21.5 | 0.0 | 0.0 | 7.3 |
| Capan-2 | 53.4 | 53.3 | 17.5 | 0.0 | 0.0 |
| H661 | 28.1 | 0.0 | 3.0 | — | 13.4 (0.0)* |
| Calu-6 | 57.8 | 18.9 | — | — | — |
| Calu-3 | 21.8 | 22.2 | — | — | — |

*13.4 is the decrease observed in Band 1 and 0.0 is the decrease observed in Band 2.

As can be seen from the Table 34, compound 10 decreases the band intensity of the alpha isoform in all the cell lines tested and does not have this effect on PKC beta 1, epsilon and zeta, which are representative of the cPKC, nPKC and aPKC groups, respectively.

A decrease in band intensity was also observed for the delta isoform, with the exception of two cell lines. This may be due to the intracellular content in PKC-δ being affected by the decrease in PKC-α intracellular level. For example, Romanova, L., et al. (1998, Biochemistry, 37, 5558-5565) demonstrated that PKC-α and PKC-δ endogenous levels are related and specifically, that overexpression of PKC-αup regulates PKC-δ levels through mRNA stabilization and enhancement of mRNA translation.

### Example 31

### Effect of Compound TIM 10 on Apoptosis in Cancer Cells

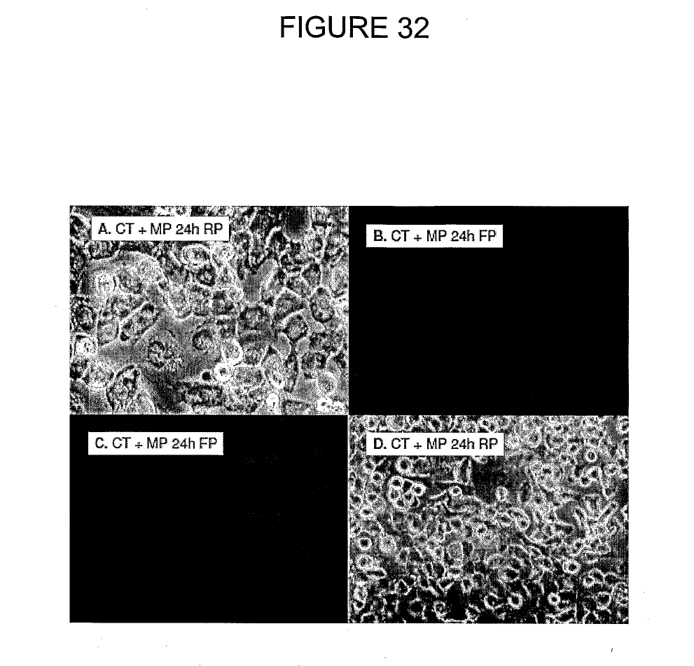

Human non-small cell lung cancer cells (H661) were submitted to endocytosis using vehicle alone or compound TIM 10 (5 mM) dissolved in PBS or in Triton X100 at 0.1% in PBS. After 24 hrs, the nuclei were stained with Hoechst reagent. The results are shown in FIGS. 32-34.

FIG. 32 shows control cells that were submitted to endocytosis using vehicle alone. The nuclei are kidney shaped and many cells are polynucleated (B and C). A and D are matching reverse phases showing suhconfluent and confluent initial cultures.

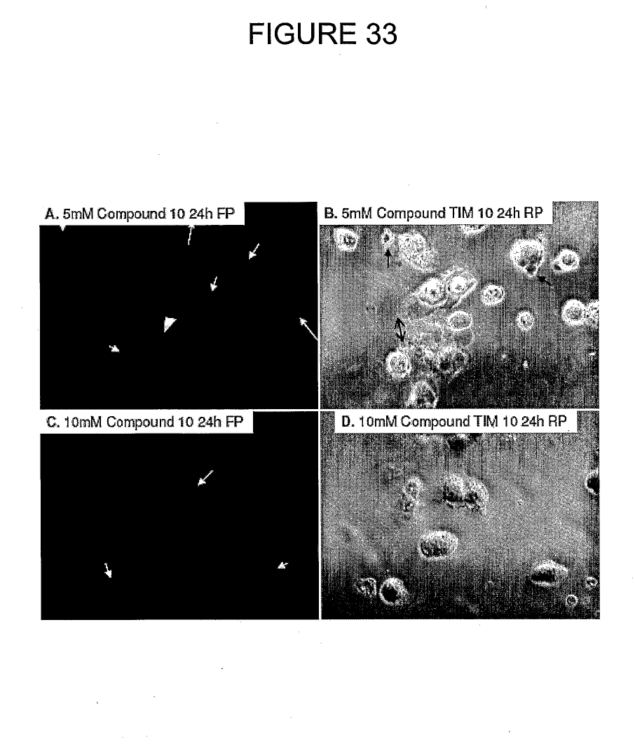

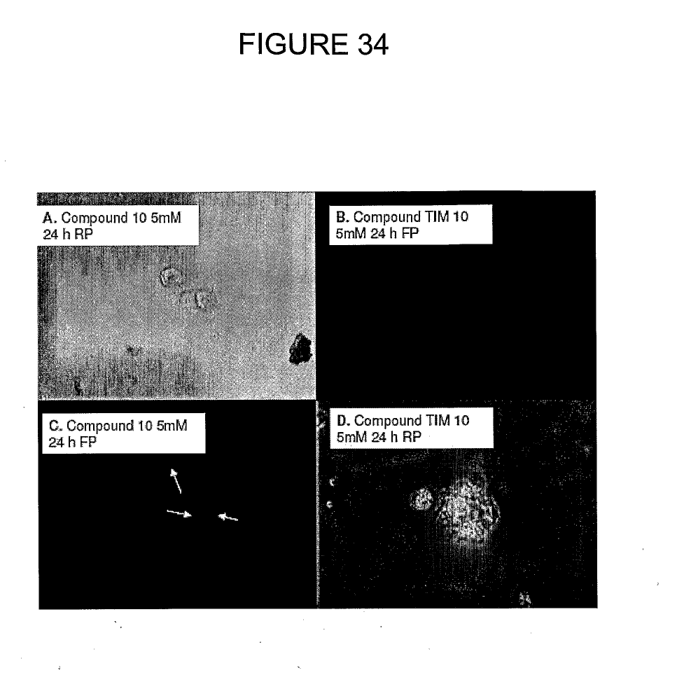

FIG. 33 shows cells after internalization of 5 mM compound TIM 10 stock dissolved in Triton X100 at 0.1% in PBS. As can be seen, the cell population is drastically decreased. Apoptosis is illustrated in A and C by chromatin packing in Hoechst stained nuclei (white arrows) and nucleus fragmentation (arrow head in A). The matching reverse phases show characteristic shedding (double arrow) and apoptotic bodies (black arrows in B). FIG. 34 shows cells after internalization of 5 mM compound TIM 10 stock dissolved in PBS. Again, the cell population is drastically decreased. Apoptosis is illustrated by nuclear fragmentation in Hoechst stained nucleus (B) and chromatin packing is observed in C (white arrows). Matching reverse phase micrographs exhibit typical apoptotic figures, namely shedding (D) and apoptotic body (black arrow).

### Example 32

### Effect of Compound TIM 10 on Cell Cycle

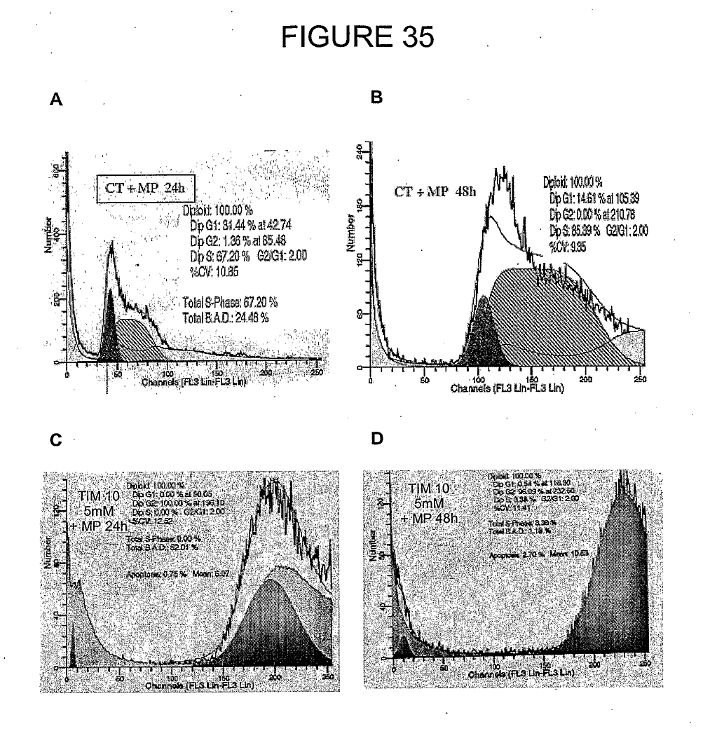

Human non-small cell lung cancer cells (H661) were submitted to pinocytic endocytosis using vehicle alone or compound TIM 10 (5 mM) and were processed for cell cycle analysis and determination of apoptosis using conventional protocols for flow cytometry (FACS). H661 cells are hexaploid and exhibit a modal number of 142 (range of 130-153). DNA however, does not present gross ultrastructural abnormalities. The abnormal chromosome number makes the study of the cell cycle difficult to analyze in these cells. The results are shown in FIG. 35 (cell percentage is on the y axis and DNA content on the x axis). (A) shows the distribution of the cells into the cell cycle phases following pinocytic treatment with vehicle alone for 24 h. The dark grey peak represents the percentage of cells in the G1 phase, S phase is shown by the hatched peak and the pale grey peak represents the G2 phase that spans a large array of cells with increasing DNA content (of 4n chromosomes). (B) shows the distribution of these control cells after 48 h and demonstrates a decrease in the G1 phase percentage of cells while more cells move to the S phase and the G2 phase, as is expected for non-synchronized proliferating cell populations. (C) shows the distribution of the cells into the cell cycle following internalization of 5 mM compound TIM 10. The cells have all moved into the G2 phase with a variable DNA content. The cells become polynucleated after treatment with compound TIM 10 (as observed under microscopic examination) and this was recognized as cell aggregates by the software employed. A small amount of apoptosis materializes in a black peak. Note that the amount of apoptosis is underestimated using this technique due to the aberrant chromosome number and DNA content of the cells. (D) shows the dramatic accumulation of the cells in the G2 phase indicating a G2 phase block caused by treatment with compound TIM 10. The apoptosis peak increased and will increase further due to the G2 block.

### Example 33

### Stable Expression of Compound TIM 16, Specificity and Efficacy Toward PKC-α Isoform

The specificity and efficacy of compound TIM 17 (see Table 28) was tested intracellularly on 3 PKC isoforms (α, δ and ε), which are the main isoforms expressed in the IMR32 neuroblastoma cell lines and neuroblastoma biopsies.

A nucleotide sequence encoding TIM 17 was designed as follows. Two oligos were prepared, a coding oligonucleotide comprised of the sequence encoding TIM 17 together with start (ATG) and stop (TAC) codons, and a complementary oligonucleotide comprised of the complementary sequence of the sense oligonucleotide.

Coding oligonucleotide:
[SEQ ID NO: 62]
5′-ATG TTT CGC CGC AAA TTT CGC CTG GGC GGC
GGC GGC GGC GGC GGC AAA GAT GCG CAG AAC CTG
ATT GGC ATT AGC ATT TGA-3′
Complementary oligonucleotide:
[SEQ ID NO: 63]
5′-TCA AAT GCT AAT GCC AAT CAG GTT
CTG CGC ATC TTT GCC GCC GCC GCC GCC GCC GCC
CAG GCG AAA -ITT GCG GCG AAA CAT-3′

The coding and complementary oligonucleotides were hybridised to form a double-stranded DNA fragment (“TIM 17 oligo”), which was then PCR amplified using the following sense and antisense primers and the protocol provided below:

Sense Primer [SEQ ID NO:64]:
5′-GGGGACAAGTTTGTACAAAAAAGCAGGCTTGATGTTTCGCCGCAA
ATTT-3′
Antisense Primer [SEQ ID NO:65]:
5′-GGGGACCACTTTGTACAAGAAAGCTGGGTCTAAATGCTAATGCCAA
T-3′

### PCR Reactions:

| | TIM 17 | | | | | | | Taq DNA |
|---|---|---|---|---|---|---|---|---|
| | oligo* | ddH₂O | Mgcl₂ | dNTPs | Sense | Antisense | 10X PCR | polymerase |
| | (ul) | (ul) | (ul) | (ul) | primer (ul) | primer (ul) | buffer(ul) | (ul) |
| 1 | 1 | 38 | 0 | 1 | 2 | 2 | 5 | 1 |
| 2 | 1 | 37 | 1 | 1 | 2 | 2 | 5 | 1 |
| 3 | 1 | 36 | 2 | 1 | 2 | 2 | 5 | 1 |

*TIM 17 oligo concentration is: 0.1 pmole/ul

### PCR Cycles:

| | | |
|---|---|---|
| 95° C. | Hold (for hot start) | |
| 95° C. | 30 seconds | |
| 30 cycles at: | 95° C. | 30 seconds |
| | 58.5° C. | 30 seconds |
| | 72° C. | 30 seconds |
| 72° C. | 5 minutes | |
| 4° C. | Hold | |

The 141 bp PCR amplified sequence encoding TIM 17 [coding strand shown in SEQ ID NO:66] was inserted into pDONRT221 vector using conventional techniques.

[SEQ ID NO: 66]
5′-GGGGACAAGTTTGTACAAAAAAGCAGGCTTGATGTTTCGCCGCAAAT
TCGCCTGGGCGGCGGCGGCGOCGGCGGCAAAGATGCGCAGAACCTGATT
GCATTAGCATTTAGACCCAGCTTTCTTGTACAAAGTGGTCCCC-3′

The synthetic TIM 17 gene was subcloned into the pREx-DEST31(7559) expression vector and transfected into IMR32 neuroblastoma cells using standard techniques. Transcription of the TIM 17 gene is under the control of a tetracycline promoter that allows gene expression to be switched on and off. Expression of the TIM 17 gene was elicited by treatment with 150 ng/ml of tetracycline for 48 h.

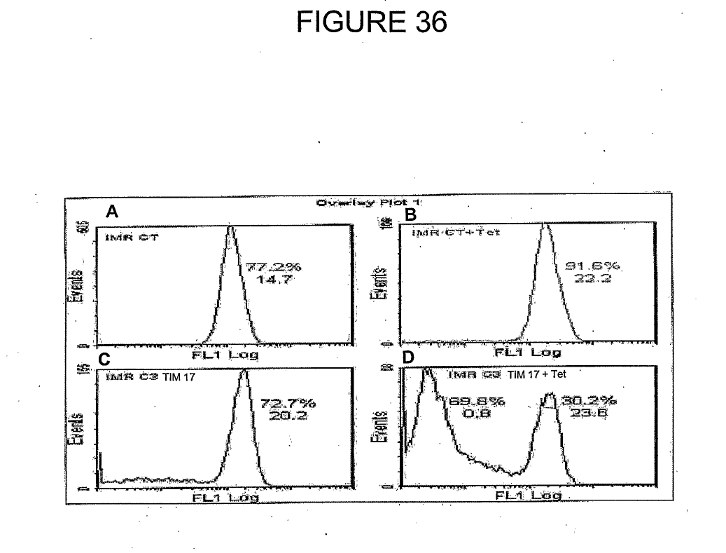

Flow cytometry analysis results for PKC-α expression are shown in FIG. 36. The data were acquired from 20,000 to 50,000 individual cells. The graph scales report the number of events measured at the peak of fluorescence while the percentage of cells positive to the specific anti-PKCalpha antibody is shown on the graphs together with the relative intensity (see number provided below the percentage). The relative intensity correlates to the mean intracellular level of PKCalpha in the cell population. The exposure of the non-transfected cells to tetracycline does not alter the PKC alpha expression (compare FIGS. 36A and B). As opposed, the exposure of the TIM 17 transfected cells to tetracycline discriminates among two cell populations (compare FIGS. 36C and D): 30% of the cells express similar levels of PKCalpha as controls while 69.8% of the population exhibits a dramatic decrease in PKC alpha cellular content which can only be attributed to the expression of TIM 17.

The expression level of PKC-α, δ and ε was also assessed by fluorescence imaging using anti-PKC antibodies from SantaCruz and secondary Alexa 488 conjugate from Invitrogen. Microscopic images were obtained from a Coulter microscope equipped with epifluorescence and the images analyzed with ImagePro+ software with the assistance of DAGE-MTI camera. The same gating was maintained constant for all images. The results indicated that a diminished cellular content in PKC-α in the IMR32 cells in which TIM 17 was expressed upon exposure to tetracycline. Expression of TIM 17 did not, however, alter the intracellular level of PKC delta and PKC epsilon isoforms. The above results were also confirmed by Western blot.

### Example 34

### Effect of Compounds TIM 10, 13, 14 and 15 on Cancer Cell Proliferation

The effect of the compounds TIM 10, 13, 14 and 15 (see Table 28) were studied on three human cancer cell lines: IMR32, neuroblastoma cell line; MDAMB 231 cancer cell line and U251, glioblastoma cell line. The increase in population size was monitored over 96 h using a modified Hoechst assay that measures the relative fluorescence intensity of the total DNA content of a population, a value that is correlated with the number of cells.

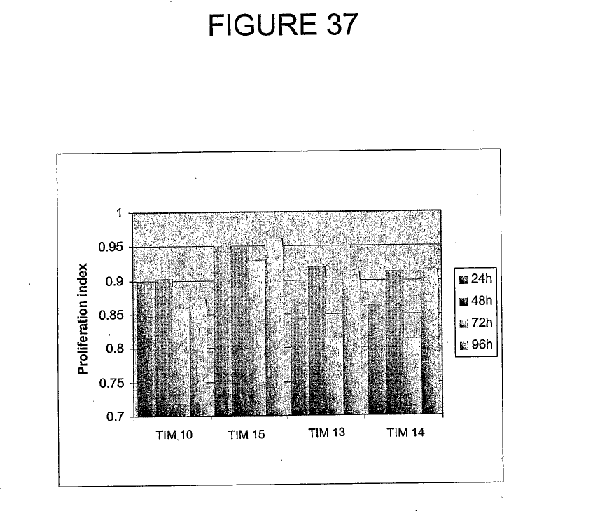

For IMR32 cells (see FIG. 37—results are reported as a ratio over matching controls) all the tested TIM compounds resulted in a decrease in cell population after 72 h, with the effect being reversed at 96 h probably due to degradation of the TIM compounds. Compounds TIM 13 and 14 showed the greatest effect, while the esterified compound (TIM 15) showed lower efficiency probably due to its degradation by esterases before it penetrates the cell.

As opposed to IMR32, proliferation of the MDAMB 231 breast cancer and U251 glioblastoma cell lines were only slightly sensitive to the TIM compounds tested. This is expected because the control of proliferation may be exerted in IMR32 through functional Gap junction channels following PKC alpha inhibition while the Connexin 43 albeit expressed in MDAMB231 does not form functional channels. Similarly, although U251 cells express PKCalpha, the main enzyme promoting survival is the AKT/PKB due to its constitutive activation in these cells. U251 cells also express Connexin43 that does not form functional channels.

### Example 35

### Effect of TPGS on the Toxicity of Compounds TIM 10 and 13 in H-69 Cells

Compounds TIM 10 and TIM 13 were shown to cause flocculation of an unidentified component of the blood serum and plasma of pigs, mice and human. In order to prevent this flocculation and increase the efficacy of the TIM compounds, advantage was taken of the properties of TPGS (alpha tocopherol polyethylene glycol succinate). The adjuvant may also protect the TIM compounds from enzymatic degradation.

The survival of NCI-H69 small cell lung cancer cells upon exposure to TIM 10 or 13 was assessed after 24 h treatment using the conventional MTT test. The compounds were added to the cells as ug/ml medium with or without TPGS. TPGS concentrations increased from 10 to 50 ug. Survival is expressed as a percentage of matching controls.

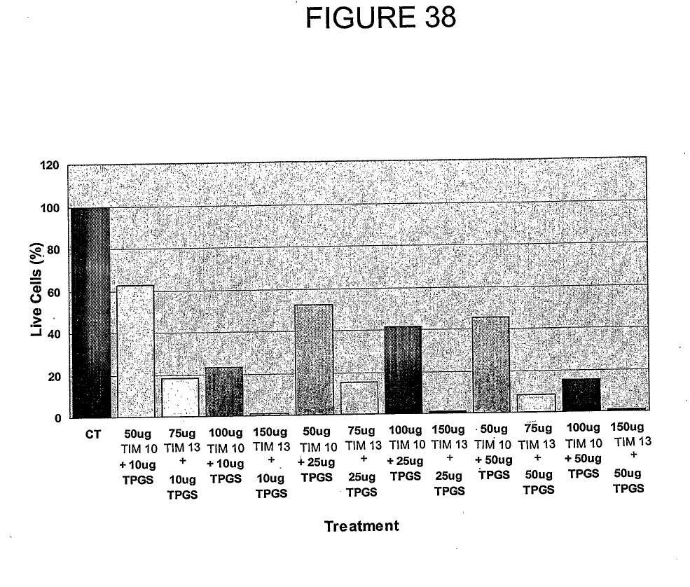

The results are shown in FIG. 38. TPGS increments did not drastically alter the 50 ug dosage of TIM 10, while the increased dosage of TIM 10 at fixed TPGS dose drastically influenced survival. The exception (100 ug TIM 10/25 ug TPGS) may be erroneous. A similar pattern was observed with TIM 13. These results suggest that the addition of TPGS increases the efficacy of the TIM compounds on NCI-H69 cell death.

### Example 36

### Effect of TIM Compounds on the Activity of Various PKC Isoforms

Compounds TIM 10, 11, 13, 14, 15, and 18-22 (see Table 28) were tested for their ability to inhibit the activity of PKC-alpha, beta 1, delta and epsilon isoforms the Kinase-Glo™ Luminescent Kinase Assay (Promega, Cat # V6712/3/4) following the manufacturer's instructions. The results are shown in Table 35.

TABLE 35
Effect of TIM Compounds on the Activity of
PKC-α, βI, -δ and -ε

| | Concentration/ | % Inhibition | | | |
|---|---|---|---|---|---|
| Compound | μM | PKC-α | PKC-δ | PKC-βI | PKC-ε |
| TIM 10 | 15.0 | 31.4 | 15.8 | 74 | −11 |
| | 30.0 | 28.6 | 22.1 | 100 | 85 |
| TIM 13 | 15.0 | 27.7 | −26.6 | 99 | 9 |
| | 30.0 | 96.6 | 1.4 | 100 | 54 |
| TIM 14 | 15.0 | 32.2 | −16 | 19 | −19 |
| | 30.0 | 23.1 | −27.8 | 27 | 14 |
| TIM 15 | 15.0 | 32.1 | 18.6 | 18.8 | 9 |
| | 30.0 | 21.8 | 21.8 | −69 | 81 |
| TIM 18 | 2.5 | 16.4 | — | — | — |
| | 5.0 | 100 | 56.3 | 76.3 | −28 |
| | 30.0 | 100 | 85.1 | 76.1 | 1 |
| TIM 19 | 5.0 | 20.3 | 41.1 | 88.2 | −6 |
| | 30.0 | 23.5 | 98.3 | 98.9 | 65 |
| TIM 20 | 5.0 | 19.8 | 46.0 | 6.7 | −9 |
| | 30.0 | 0 | 45.4 | 15.1 | −29 |
| TIM 11 | 5.0 | 41.2 | 29.0 | 11.1 | −10 |
| | 30.0 | 100 | 100 | 17.5 | 27 |
| TIM 21 | 2.5 | — | 26.2 | — | — |
| | 5.0 | 46.6 | 100 | 11 | −5 |
| | 15.0 | 64.3 | 100 | — | — |
| | 30.0 | 95.2 | 100 | 13.9 | 52 |
| TIM 22 | 2.5 | 42.8 | 26.5 | 21.9 | — |
| | 5.0 | 100 | 100 | — | 60 |
| | 30.0 | 100 | 96.7 | 30.9 | 92 |

Compounds TIM 10, 13, 14 and 15 all share the same core structure. TIM 13, however, additionally contains the PTD peptide, TIM 14 contains the Fc peptide and TIM 15 contains an esterified aspartate residue. The PTD peptide targets the TIM to the cytoplasm, the Fc peptide targets the TIM to the nucleus and esterification increases the cell permeability of the TIM.

As can be seen from Table 35, the effects on PKC alpha are similar for compounds TIM 10, 13, 14 and 15. The same “saturation” of activity was observed for all four compounds with the exception of TIM 13, which becomes very potent at high concentration. PKC delta is inhibited by both TIM 10 and 15, but not by TIM 13 and 14. PKC beta 1 is dramatically inhibited by TIM 10 and TIM 13. The two other compounds are less inhibitory, and PKC epsilon is only sensitive at the higher concentration to TIM 10, 13 and 15. It is important to note that the differences in activity observed in this assay may be somewhat artefacual because there is no enzyme that can cleave the added moieties in the assay. In cells different results may be observed.

TIM 18 is a very potent inhibitor of PKC alpha, a result that was expected. It also inhibits the activity of the PKC delta and beta1 but has no effect on PKC epsilon. TIM 19 was expected to discriminate the atypical PKC from the two other groups of PKCs due to the affinity of its PRE component and activity of its PKI component. As expected this compound was very potent against PKC beta1. At low concentration, it inhibits PKC beta while having little effect on the classical and novel groups.

As expected from the activity of its PRE and PKI constituents, TIM 20 showed good specificity for PKC delta. TIM 11 is a good inhibitor for PKC alpha and delta to a lesser extent. This was expected due to the presence of the PRE 4 moiety.

TIM 21 is an exceptionally good inhibitor of PKC delta and to PKC alpha at a lesser extent while it is not active on PKC beta1. TIM 22 was designed with the intention that it would strongly inhibit PKC alpha as it does, but it is also a good inhibitor of PKC delta.

The specificity of the various TIM compounds observed in this experiment likely results essentially from the PKI component since the experimental set up does not permit the PRE component to be very effective.

### Example 37

### Sublocalization of TIM 10 and 13 in LS-180 and IMR-32 Cells

A Biotin-Avidin system was used to determine the intracellular sublocalization of TIM 10 and 13. For this experiment, modified versions of TIM 10 and 13 were prepared that were biotinylated at a Lysine residue.

Avidin conjugated with fluorescent FITC (fluorescein isothiocyanate) was used to detect the biotinylated compounds. The bound FITC-avidin conjugate can be visualised by green fluorescence at excitation filter 496 nm and emission 525 nm.

LS180 cells were treated with increasing concentrations of biotinylated TIM 10 or TIM 13 for 24 h and 72 h. After completion of each incubation period the cells were washed and fixed with 4% paraformaldehyde for 30 min and permeabilized with 0.1% triton X-100 for 10 minutes.

The cells were washed 3× with PBS and were incubated with avidin conjugated with FITC1:500 dilutions in PBS for 1 h at room temperature. The cells were washed with PBS and images were taken with the DAGE-MTI camera with the assistance of Image PRO Plus 4.5 software.

At both 24 and 72 hours, the biotinylated TIM 10 was observed to localize only on the plasma membrane of the cells, whereas TIM 13 was observed to localizes mainly in the cytoplasm of the cells, as expected.

IMR32 cells were treated with biotinylated TIM 10 or 13 as described above for the LS180 cells. Biotinylated TIM 10 was observed to also label the membrane of IMR32 cells. In some cells the entire membrane was coated by TIM 10. In most cells however, a punctual label on the membrane was observed that suggests a specificity of TIM 10 binding in this cell line.

TIM 13 after 24 h exposure was observed to localize inside the cytoplasm as expected. Some mottling in the cytoplasm was also observed that suggests binding to specific cytoplasmic molecules. Some accumulation of the compound in a perinuclear location in some cells was observed as well as some membrane labeling. After 48 h treatment diffuse localization of the TIM 13 compound in the cytoplasm was observed.

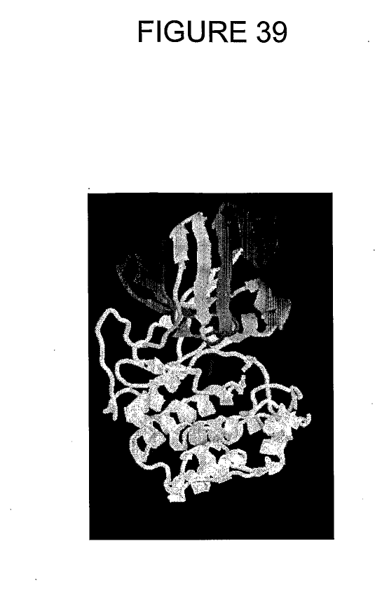

The following Examples 38 to 40 below provide in vivo data for a compound of the invention TIM 10 (or “PhGalpha1”). A computer model for PKC-α (shown in representative form only in FIG. 39) was used to develop a rigorous screening process for peptide “fragments”, with the most potent fragments incorporated into the final design of the targeted PKC-α inhibitor PhGalpha1 (TIM 10). The results can be summarised briefly as follows:

Time to Establish and Grow Drug Resistant (MDR) Colon Cancer More than Doubled

Colon cancer tumour establishment and growth (measured in days) was delayed by an average of 100% in mice receiving PhGalpha1 in combination with a widely used chemotherapeutic agent versus mice in a non-treated control group.

### Delay in Establishment of Metastatic Breast Cancer

Breast cancer tumour establishment was delayed by an average of 60% in mice receiving PhGalpha1 versus mice in a non-treated control group.

### Breast Cancer Tumours Rendered Benign

Tumour analysis revealed that untreated tumour cancer cells exhibited aggressive re-growth, whereas treated tumour cancer cells were fully differentiated (i.e. mature) and essentially benign.

### No Toxicity

PhGalpha1 was administered to fifty mice in 72 hour cycles over periods of up to 75 days. No evidence of toxicity was observed or evident in pathology analysis.

### Example 38

### Effect of Compound TIM 10 on the Establishment and Growth of Drug Resistant (MDR) Colon Cancer

For this Example and Example 39, nude mice, CD1/CD1 outbred strain, that were subcutaneously injected with cancer cells to form tumours were used. The final cancer models selected are human in origin and were not passaged at any time in a rodent. The cell lines were: LS180 human colorectal adenocarcinoma cells (this Example) and MDA-MB-231 human mammary adenocarcinoma (Example 39). The cell lines were injected subcutaneously into the left flank at a concentration of 5×10⁶ per mouse. A control group consisting of N=5 mice was used for each type of cancer model selected. The control mice were injected with 5×10⁶ cells subcutaneously into the left flank. An additional control group of N=5 received no injection of any kind and was used as a baseline for body weight and behavioural measures.

The effect of TIM 10 on timing of tumour appearance (M1) or transition from M1 (2×2 mm) to M2 (7×5 mm) or increasing tumour cell differentiation and protein expression in comparison to untreated cancer mice was investigated. TIM 10 was delivered into the left flank subcutaneously (prior to appearance of the tumour) or intratumourally (once tumour was established) every 72 hrs at a dose of 5 mg/kg. Mice injected with LS180 cells received an additional treatment of 1 mg/kg doxorubicin via the tail vein at a dosing schedule known to induce multidrug resistance (MDR).

LS180 Colon cancer mice were divided into 4 treatment categories: (1) traditional chemotherapy like doxorubicin used at a sub-therapeutic dose to induce MDR; (2) 5 mg/kg of TIM 10; (3) simultaneously administered doxorubicin and TIM 10, labelled combination #1, and (4) 10 days of doxorubicin treatment followed by continued doxorubicin treatment in combination with 5 mg/kg TIM 10 labelled combination #2. TIM 10 was administered every 72 hrs. The study was carried out over a period of 75 days.

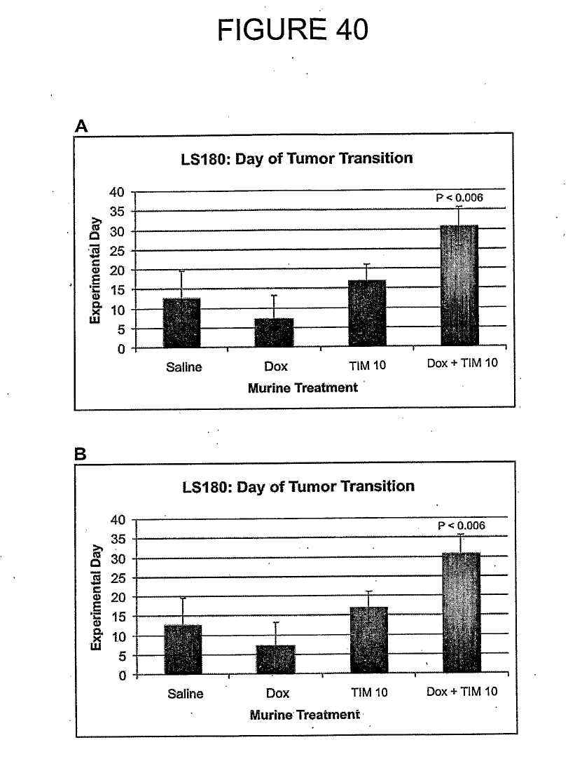

Tumour establishment in mice receiving TIM 10 and a pre-treatment with doxorubicin (chemotherapeutic drug used to trigger drug resistance) was delayed an average of 14 days compared to a control group receiving saline (28 days for the treated group compared with 14 days to reach tumour establishment for saline group—see FIG. 40A). The approximate 14-day difference seen with the treated cohort represents a 100% delay in tumour establishment versus the control group (p<0.03).

Establishment of tumours was deemed to occur at a size of 1-2 mm×2 mm (M1 stage).

Following establishment, LS180 tumour growth was monitored until it reached the size of approximately 4-5 mm×7-8 mm (M2 stage). Tumour growth following establishment in the cohort receiving TIM 10 and a pre-treatment with doxorubicin occurred after 31 days compared with 13 days for the control group. Overall, from initial injection to the M2 stage took 58 days for the doxorubicin/TIM 10 treated group compared to 26 days for the control group (see FIG. 40B).

The results of this test are consistent with earlier in vitro tests showing that TIM 10 has a potent effect ameliorating MDR and supports the utility of TIM 10 for increasing the efficacy of cytotoxic chemotherapy agents now in clinical use.

### Example 39

### Effect of Compound TIM 15 in Delaying Establishment of Metastatic Breast Cancer

The effect of TIM 15 on MDA-MB-231 Breast Cancer mice was investigated following the procedure described in Example 38. Mice received MDA-MB-231 cells previously treated with TIM 15 followed by direct tumoural injection (or injection into the cell vicinity) of 5 mg/kg of TIM 15 every 72 hrs. The study was carried out over a period of 75 days.

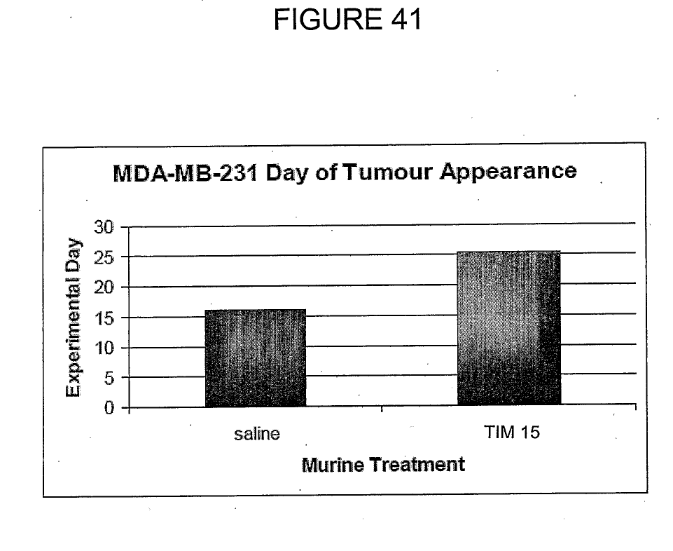

Tumour establishment (M1—defined as described in Example 38) was delayed an average of 9 days in mice treated with PhGalpha1 versus control mice receiving saline (25 days for the treated group compared with 16 days to reach tumour establishment for saline group—see FIG. 41). The approximately 9-day difference seen with the treated cohort represents an approximate 60% delay in tumour establishment versus the control group (p<0.001).

Pathology analysis of tumours revealed that, in 3 of 5 samples, tumours in the cohort treated with PhGalpha1 were composed of up to 90% fatty tissue and with as little as 10% solid tumour, whereas 4 out of 5 tumours from the control group were solid tumours with no fatty tissue. Tumourogenicity of tumour cells samples was assessed in a limited study. Tumour cells removed from the untreated group readily grew using a standard agar protocol, indicating continued proliferative characteristics. However, tumour cells taken from the treated group would not grow or proliferate. Fatty tissue cells surrounding the small solid tumour were found to be fully differentiated and essentially benign.

### Example 40

### Toxicity of Compound TIM 10

There was no evidence of toxicity in mice given a regular 72-hour dosing schedule of TIM 10 over a 75-day test period (repeat-dose toxicity study). A dosage of 25 micrograms per mouse was provided to a cohort of mice by subcutaneous injection. No physiological, behavioral or external signs of toxicity were observed. Follow-up pathological and histological organ studies also showed no signs of toxicity.

Acute toxicity studies were performed on mice using three different delivery routes: IV (tail), topical and oral. No toxic effects were observed at any dose when compound TIM 10 was administered topically or orally. The LD₅₀ obtained from the IV study was determined to be 750 μg-1 mg per mouse. The “no observed adverse effect level” was determined to be 250 μg per mouse, approximately twice the concentration of TIM 10 provided to mice in the studies discussed above. Subsequent pathology of organs showed no systemic toxicity.

In vitro studies to investigate the effect of compounds TIM 10, 13, 14 and 15 on peripheral blood lymphocytes survival and blastogenic response to mitogens using primary lymphocytes isolated from the blood of pigs, mice and human subjects indicated that the compounds do not induce apoptosis of the peripheral blood lymphocytes and do not drastically limit the blastogenic response. The largest effect was observed with the TIM compound incorporating the PTD peptide, TIM 13.

This result indicates that a wide dose range is available for animal and human clinical studies and is an important milestone for pre-clinical development of TIM 10. In addition, TIM 10 is specific to PKCalpha (see results above), has potent efficacy and low toxicity, is not an ATP-competitive inhibitor (i.e. may be more specific and less toxic than ATP inhibitors), directly inhibits PKCalpha rather than targeting PKC-RACK protein binding; and is a peptide drug, which facilitates its administration alongside other chemotherapeutic regimens.

### Example 41

### Effect of Compound TIM 10 on Expression of PKC-α, MRP-1 and P-gp

The LS180 colorectal cancer cell line has constitutive levels of multi-drug resistance (MDR) related proteins, reliably enhanced by treatment with doxorubicin. Flow cytometry studies (see FIG. 28A) using fluorescent calcein-AM were used to analyze the effect of TIM 10 on MDR activity. Treatment of LS180 cells with a widely used chemotherapeutic, doxorubicin (50 ng/mL), increased MDR efflux activity. When doxorubicin treated cells then received TIM 10 (5 μM), a reduction in MDR channel efflux activity was demonstrated.

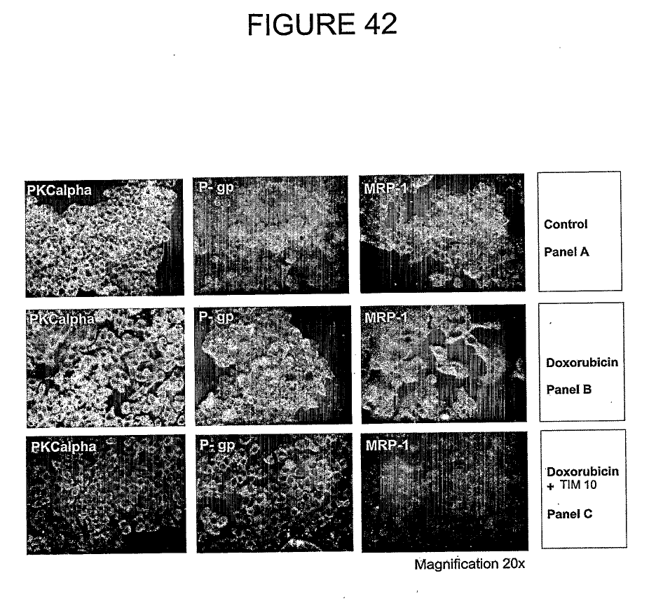

In vitro studies have also demonstrated that TIM 10 downregulates the two dominant MDR proteins and PKCalpha in LS180 cells. Immunocytochemistry (FIG. 42) revealed that expression of MDR efflux pump proteins, P-gp and MRP-1, as well as PKCalpha, was decreased following administration of TIM 10 to LS180 cells.

Panel A in FIG. 42 shows LS180 controls, showing constitutive levels of PKCalpha, P-gp and MRP-1. In Panel B, cells treated with doxorubicin (50 ng/mL) show an increase in MDR protein expression (and PKCalpha) versus Panel A controls (expected, since doxorubicin increases the MDR phenotype in LS180 cells). In Panel C, cells receiving TIM 10 (5 μM)+doxorubicin show an observable decrease in MDR protein expression.

Similar effects on PKC-alpha expression were observed with compounds TIM 13 and 15 in that these compounds consistently diminished the labeling intensity of the cells by the PKC alpha specific antibody even following increased synthesis of PKC alpha under exposure of the cells to doxorubicin. Compound TIM 14, which contains the Fc peptide, did not affect PKC-alpha expression. This result, however, was to be expected since PKCalpha is found in the cytoplasm and the plasma membrane and the Fc peptide targets the TIM to the nucleus. Similar results were observed in Caco 2 colon cancer cells treated with TIM 10, 13, 14, or 15.

### Example 42

### In Vivo Dose-Response Study with Compound TIM 10

Nude mice, CD1/CD1 outbred strain, were used. A control group consisting of N=4 mice were maintained strictly as a baseline for body weight and behaviour control group. All other mice in the study received 5×10⁶ LS180 cells subcutaneously injected into the left flank.

Each cancer model received direct tumoural injection of 2.5, 5.0, 7.5 or 10.0 mg/kg TIM 10 or intravenous doxorubicin (1 mg/kg) or a combination thereof. Prior to the appearance of the tumour, TIM 10 was injected in the vicinity of the injected cancer cells. LS180 colon cancer mice received direct tumoural injections (or injection into the vicinity) and were divided into 4 broad treatment categories: (1) physiological saline treatment; (2) doxorubicin only treatment; (3) TIM 10 treatment at four different doses, and (4) doxorubicin only for 10 days followed by combined treatment with TIM 10 administered every 72 hrs. The total number of nude mice required was 84 and the duration of the study was 60 days.

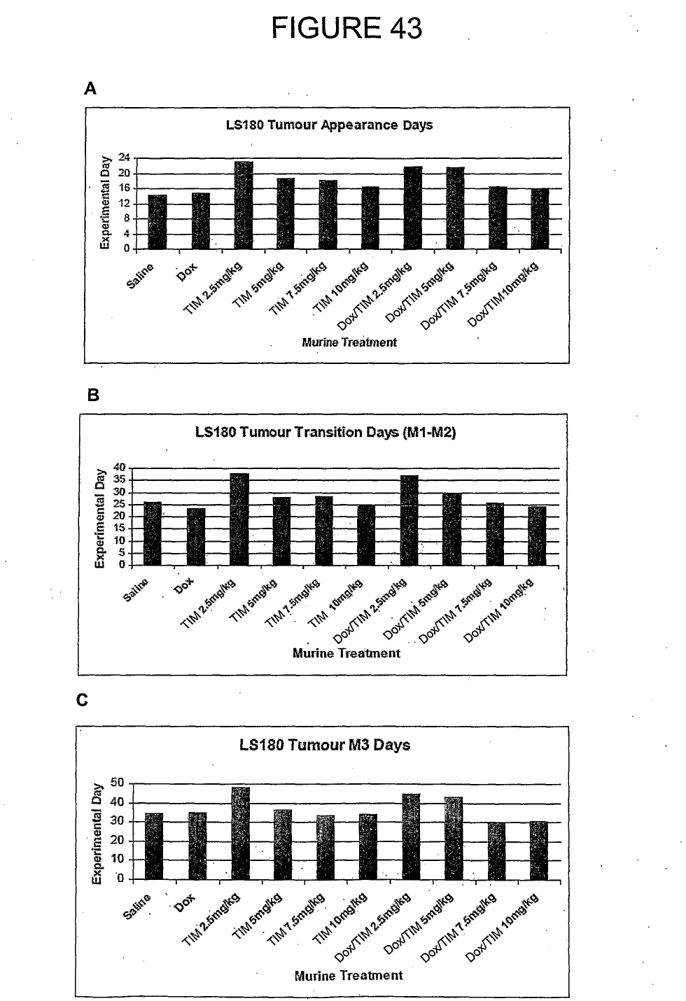

Tumours were measured on a daily basis with calipers. FIG. 43A shows the mean day of tumour appearance (M1—defined as a tumour of approximately 2×2 mm in size) across the ten groups. FIG. 43B shows the mean day of tumour transition (M1 to M2—defined as a tumour of approximately 7×5 mm in size) across the ten groups, and FIG. 43C shows the mean day of marked tumour progression (M3—defined as a tumour of approximately 12×9 mm in size) across the ten groups.

Across all doses and treatments for each stage of tumour development the most efficacious dose of TIM 10 to delay tumour establishment (M1) and progression (M2 and M3) was 2.5 mg/kg; the lowest dose administered. This observation was confirmed using a statistical trend analysis procedure. In all cases the peak efficacy of the compound was at 2.5 mg/kg and the effect is statistically significant versus the control cohorts (p<0.039).

### Example 43

### Protein Analysis of Tumour Samples

Tumour samples were collected four times throughout the duration of the study described in Example 43 in order to examine any potential time course of changes in expression of PKCα and the MDR proteins Pgp and MRP-1 as the tumour developed. LS180 tumour samples from each group were dissociated using a standard dispase protocol. The three proteins were examined separately by flow cytometry across the exposure days to TIM 10 (Day 30, Day 40 and Day 60).

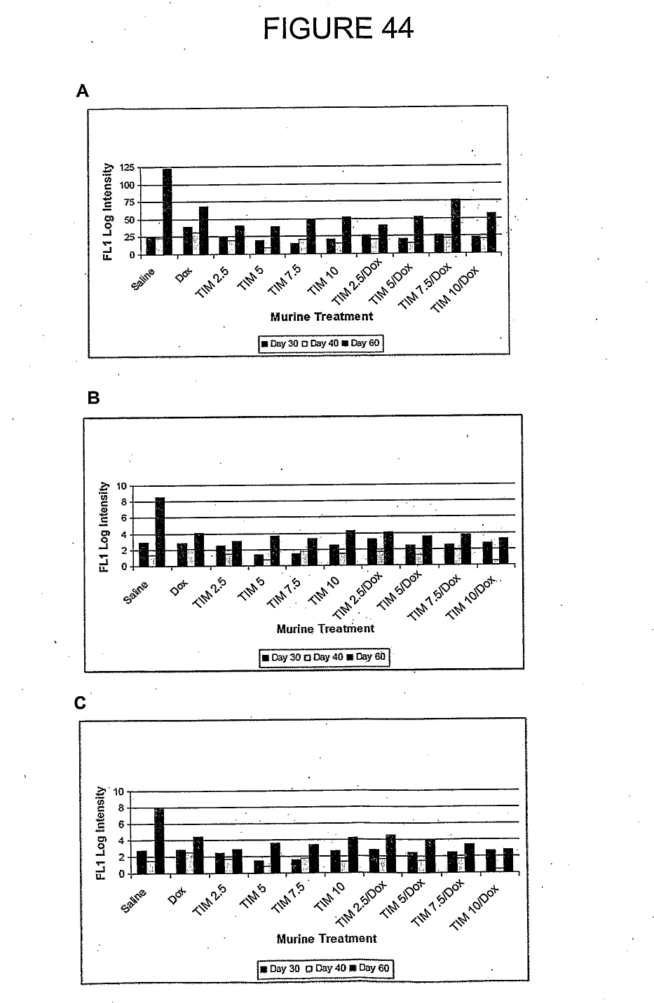

The results are shown in FIG. 44. As can be seen from FIG. 44A, on Day 30, the doxorubicin treated tumours expressed the highest level of PKCα protein and the lowest levels were detected from the tumours that had been treated with 5 mg/kg or 7.5 mg/kg of TIM 10. By Day 40, PKCα expression continued to be highest in the doxorubicin treated tumours and lowest in the tumours that received 5 mg/kg of TIM 10. PKCα protein expression is at its highest level across all the treatments by Day 60 of the study. Saline treated tumours express the highest level of PKCα and TIM 10 2.5 mg/kg, TIM 10 5 mg/kg and TIM 10 2.5/Dox express the lowest levels of PKCα protein. In summary, tumours treated with 5 mg/kg of TIM 10 consistently expressed the lowest amount of PKCα protein across the entire time course of the in vivo study.

On Day 30, Gli2.5/Dox treated tumours expressed the highest level of Pgp protein (see FIG. 44B). The lowest levels were detected from the tumours that had been treated with 5 mg/kg or 7.5 mg/kg of TIM 10. By Day 40, Pgp expression is highest in the doxorubicin treated tumours and lower across the board in all tumours that received TIM 10. Pgp protein expression is at its highest level across all the treatments by Day 60 of the study. Saline treated tumours express the highest level of Pgp and tumours treated with any dose of TIM 10 express the lowest levels of Pgp protein. In summary, tumours treated with TIM 10 at 2.5 mg/kg and 5 mg/kg expressed the lowest amount of Pgp protein across the entire time course of the in vivo study.

FIG. 44C shows that on Day 30, all tumours expressed a consistent level of MRP-1 protein expression with the exception of TIM 10 at 5 mg/kg and 7.5 mg/kg which were considerably lower than the rest of the treatments. By Day 40, there was a substantial drop in MRP-1 expression across all treatments with the doxorubicin treated tumours maintaining the highest level of MRP-1 expression. Overall MRP-1 protein expression is at its highest level across all the treatments by Day 60 of the study. Saline treated tumours express the highest level of MRP-1 and tumours treated with TIM 10 at 2.5 mg/kg express the lowest levels of MRP-1 protein.

### Example 44

### CD44 and CD66 Biliary Glycoprotein Expression

Expression of tumour associated cell surface antigens is a reflection of the state of cell differentiation of tumour cells. The cells from the tumour samples taken as described in the preceding Examples 42 and 43 were isolated and grown in MEM medium. The cells were labeled with monoclonal antibody clone B6.2/CD66 conjugated with R-PE and were analyzed by flow cytometry.

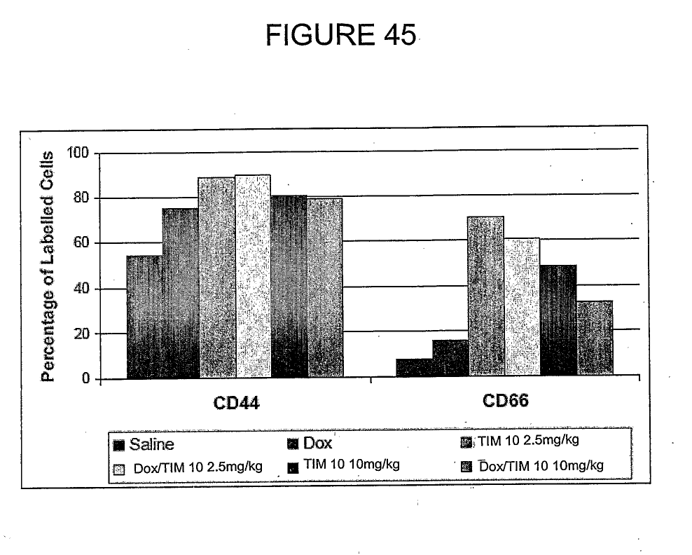

The results are shown in FIG. 45. Data are expressed as the percentage of positive cells expressing CD66 antigen in the tumour cells population. CD66 is a carcinoembryonic antigenrelated protein called as biliary glycoprotein (CEACAM1). It is known that CEACAM1 is present in normal cells but its expression dramatically reduces in early phase of colon cancer. Reintroduction of these proteins in cells which had lost their expression restores a normal-like phenotype. Cells expressing CD66 provide an indicator of the degree of differentiation in cells isolated from the tumour biopsies. The second antibody used was also a monoclonal antibody against extracellular matrix protein CD44 which recognizes 80-95 kDa glycosylated type I transmembrane protein, also known as phagocytic glycoprotein-1. The cells isolated from tumours were labeled with CD44 monoclonal antibody conjugated with FITC and cells were analyzed by flow cytometry (see FIG. 45). The simultaneous analysis of both antibodies is a reliable measure of the state of differentiation of colon cancer adenocarcinoma cells.

The data demonstrate that cells isolated from tumours treated with TIM 10 at 2.5 mg/kg alone and in combination with doxorubicin have shown the highest level of differentiation as compared to the cells isolated from biopsies treated with saline, doxorubicin alone and higher dosages of TIM 10 and TIM 10 combined with doxorubicin.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

Although the invention has been described with reference to certain specific embodiments, various modifications thereof will be apparent to those skilled in the art without departing from the spirit and scope of the invention as outlined in the claims appended hereto.

1. A targeted protein kinase C (PKC) inhibitor comprising an inhibitor moiety that is capable of inhibiting the activity of a PKC operatively associated with a peptide of about 5 and about 30 amino acid residues in length, said peptide having a sequence of general formula (I), or the retro form thereof:
X—[(HY—HB)ₙ-linker]ₘ-(HB—HY)₂—HB—(HY)ₘ—Z (I) (SEQ ID NO:67)
wherein:
HY represents 1 to 4 amino acid residues selected from the group of Ala, Gly, Ile, Leu, Phe and Val;
HB represents 1 to 4 amino acid residues selected from the group of: Arg, Asn, Asp, Glu, Gln, Lys and Ser;
“linker” represents 1 to 4 Gly residues;
n is 1, 2 or 3;
m is 0 or 1;
X represents the N-terminus of the peptide or a modified version thereof, and
Z represents the C-terminus of the peptide or a modified version thereof.

2. The targeted PKC inhibitor according to claim 1, wherein said inhibitor moiety and said peptide are operatively associated via a spacer.

3. The targeted PKC inhibitor according to claim 2, wherein said spacer is an amino acid sequence between about 1 to about 18 amino acid residues in length.

4. The targeted PKC inhibitor according to any one of claims 1, 2 or 3, wherein said peptide has a sequence of general formula (II), or the retro form thereof:
X—[(HY—HB1)ₙ-linker]ₘ-(HB—HY)₂—HB2-(HY)ₘ—Z (II)
wherein:
HB1 represents 1 to 3 amino acid residues selected from the group of: Arg, Asn, Asp, Glu, Gln, Lys and Ser; and
HB2 represents 1 or 2 amino acid residues selected from the group of: Arg, Asn, Asp, Glu, Gln, Lys and Ser.

5. The targeted PKC inhibitor according to any one of claims 1, 2 or 3, wherein said peptide has a sequence of general formula (III), or the retro form thereof:
X—(HB—HY)2-HB2-(HY)ₘ—Z (III)
wherein:
HB2 represents 1 or 2 amino acid residues selected from the group of: Arg, Asn, Asp, Glu, Gln, Lys and Ser.

6. The targeted PKC inhibitor according to any one of claims 1, 2 or 3, wherein said “linker” represents 1 to 3 Gly residues.

7. The targeted PKC inhibitor according to any one of claims 1, 2 or 3, wherein said “linker” represents 1 to 2 Gly residues.

8. The targeted PKC inhibitor according to any one of claims 1, 2, 3, 4, 5, 6 or 7, wherein said peptide comprises one or more non-naturally-occurring amino acids.

9. The targeted PKC inhibitor according to any one of claims 1, 2, 3, 4, 5, 6 or 7, wherein said peptide comprises one or more modified peptide bonds.

10. The targeted PKC inhibitor according to any one of claims 1, 2, 3, 4, 5, 6 or 7, wherein said peptide comprises one or more D-amino acids.

11. The targeted PKC inhibitor according to claim 1, wherein said peptide comprises an amino acid sequence selected from the group of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ED NO:34 and SEQ ID NO:35, or the retro, inverso, or retro-inverso form thereof.

12. The targeted PKC inhibitor according to claim 1, wherein said peptide comprises an amino acid sequence selected from the group of: SEQ ID NO:1, SEQ ID NO:2, SEQ ID NO:3, SEQ ID NO:4, SEQ ID NO:5, SEQ TD NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ TD NO:18, SEQ ID NO:19, SEQ ID NO:20, SEQ ED NO:21, SEQ ID NO:22, SEQ ID NO:23, SEQ ED NO:24, SEQ ID NO. 25, SEQ ID NO:26, SEQ ID NO:27, SEQ ID NO:28, SEQ ID NO:29, SEQ ID NO:30, SEQ ED NO:31, SEQ ED NO:32, SEQ ID NO:33, SEQ ID NO:34 and SEQ ID NO. 35.

13. The targeted PKC inhibitor according to claim 1, wherein said peptide comprises an amino acid sequence selected from the group of: SEQ ID NO:1, SEQ ID NO:5, SEQ ID NO:11, SEQ ID NO:12, SEQ ED NO:13, SEQ ID NO. 14, SEQ ID NO:16, SEQ ID NO:17, SEQ ID NO:18, SEQ ID NO:19, SEQ ID NO:20, SEQ ED NO:21 and SEQ ID NO:22.

14. The targeted PKC inhibitor according to claim 1, wherein said peptide comprises a sequence as set forth in SEQ ID NO:2 or SEQ ID NO:13.

15. The targeted PKC inhibitor according to any one of claims 1, 2, 3, 4, 5, 6, 7, 8, 9, 10, 11, 12, 13 or 14, wherein said inhibitor moiety is a compound of general formula IX:
(C1)J(M)-N_{y}B_{z}AₓB_{y}N_{y}Bₓ (IX)
wherein:
C1 is NₓB_{y}(A/N)ₓ B_{y}N_{y} and is attached to J by a peptide bond from the N- or C-terminus of C1;
J is 1-4 amino acid residues selected from the group of: Cys, Lys and His;
M is absent or an ATP mimetic moiety optionally linked to an amino acid selected from the group of Ile, Leu, Val or Gly and is attached to J via the side chain or the N-terminus of one of the Lys residues of J or the N-terminus of one of the Cys residues of J;
each N is independently Ala, Ile, Leu, Val or Gly;
each B is independently Arg, Lys or Tyr; and
each A is independently Phe, His or Trp;
each x is independently 0-1;
each y is independently 0-2;
z=0-3, and
the sequence N_{y}B_{z}AₓB_{y}N_{y}Bₓ is 2 or more amino acids in length,
wherein:
when J comprises one or no Cys residues, the compound of Formula (IX) comprises a single peptide chain and C1 is attached to the N-terminal amino acid of J via a peptide bond from the C-terminus of C1, and
when J comprises two or more Cys residues, at least two of the Cys residues are linked by a disulphide bond and the compound of Formula (IX) thereby comprises a first peptide chain comprising a first of said at least two Cys residues and C1, and a second peptide chain comprising a second of said at least two Cys residues and the sequence —N_{y}B_{z}AₓB_{y}N_{y}Bₓ, and
wherein if M is absent, the sequence —NyB_{z}AₓByNyBₓ contains at least one of Phe or Trp.

16. The targeted PKC inhibitor according to claim 15, wherein said inhibitor moiety is a compound of Formula (X):
(C1)J(M)-N_{y}B_{z}AₓB_{y}N_{y} (X)
wherein:
C1 is NₓB_{y}(A/N)ₓ B_{y}N_{y} and is attached to J by a peptide bond from the N- or C-terminus of C1;
J is 1-4 amino acid residues selected from the group of: Cys, Lys and His;
M is absent or an ATP mimetic moiety optionally linked to an amino acid selected from the group of Ile, Leu, Val or Gly and is attached to J via the side chain or the N-terminus of one of the Lys residues of J or the N-terminus of one of the Cys residues of J;
each N is independently Ala, Ile, Leu, Val or Gly;
each B is independently Arg, Lys or Tyr; and
each A is independently Phe, His or Trp;
each x is independently 0-1;
each y is independently 0-2;
z=0-3, and
the sequence N_{y}B_{z}AₓB_{y}N_{y} is 2 or more amino acids in length, and
wherein:
when J comprises one or no Cys residues, the compound of Formula (I) comprises a single peptide chain and C1 is attached to the N-terminal amino acid of J via a peptide bond from the C-terminus of C1, and
when J comprises two or more Cys residues, at least two of the Cys residues are linked by a disulphide bond and the compound of Formula (I) thereby comprises a first peptide chain comprising a first of said at least two Cys residues and C1, and a second peptide chain comprising a second of said at least two Cys residues and the sequence —N_{y}B_{z}AₓB_{y}N_{y}Bₓ.

17. The targeted PKC inhibitor according to claim 15, wherein said inhibitor moiety is a compound of Formula (XI):
(C2)J(M)-N_{y}B_{z}AₓB_{y}N_{y} (XI)
wherein:
C2 is B_{y}(A/N)ₓ B_{y}N_{y} and is attached to J by a peptide bond from the N- or C-terminus of C2;
J comprises two Cys residues and optionally 1-2 residues selected from His and Lys, the Cys residues are linked by a disulphide bond and the compound of Formula (I) thereby comprises a first peptide chain comprising a first of said two Cys residues and C2, and a second peptide chain comprising a second of said two Cys residues and the sequence —N_{y}B_{z}AₓB_{y}N_{y}Bₓ,
M is an ATP mimetic moiety optionally linked to an amino acid selected from the group of Ile, Leu, Val or Gly and is attached to J via the N-terminus of one of the Cys residues;
each N is independently Ala, Ile, Leu, Val or Gly;
each B is independently Arg, Lys or Tyr; and
each A is independently Phe, His or Trp;
each x is independently 0-1;
each y is independently 0-2, and
z=0-3.

18. The targeted PKC inhibitor according to claim 15, wherein said inhibitor moiety is a compound of Formula (XII):
NₓB_{y}(A/N)ₓB_{y}N_{y}-J(M)-NyB_{z}AₓB_{y}N_{y}Bₓ (XII)
wherein:
J is 1-2 Lys residues or a Cys residue;
M is absent or is an ATP mimetic moiety attached to J via the side chain of one of the Lys residues or the N-terminus of the cysteine residue;
each N is independently Ala, Ile, Leu, Val or Gly;
each B is independently Arg, Lys or Tyr; and
each A is independently Phe, His or Trp;
each x is independently 0-1;
each y is independently 0-2, and
z=0-3.

19. The targeted PKC inhibitor according to claim 15, wherein said inhibitor moiety is a compound selected from the group of:

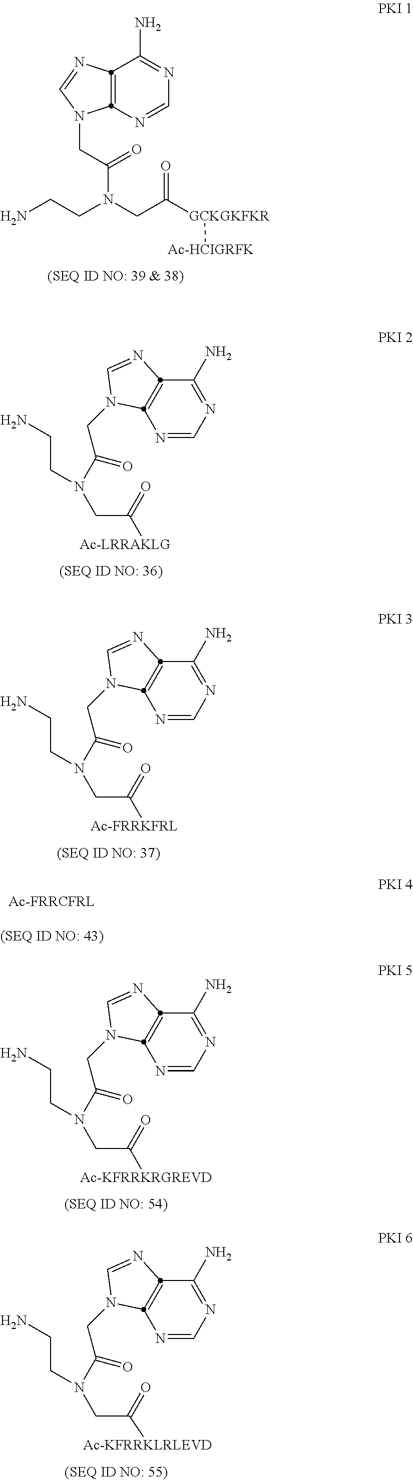

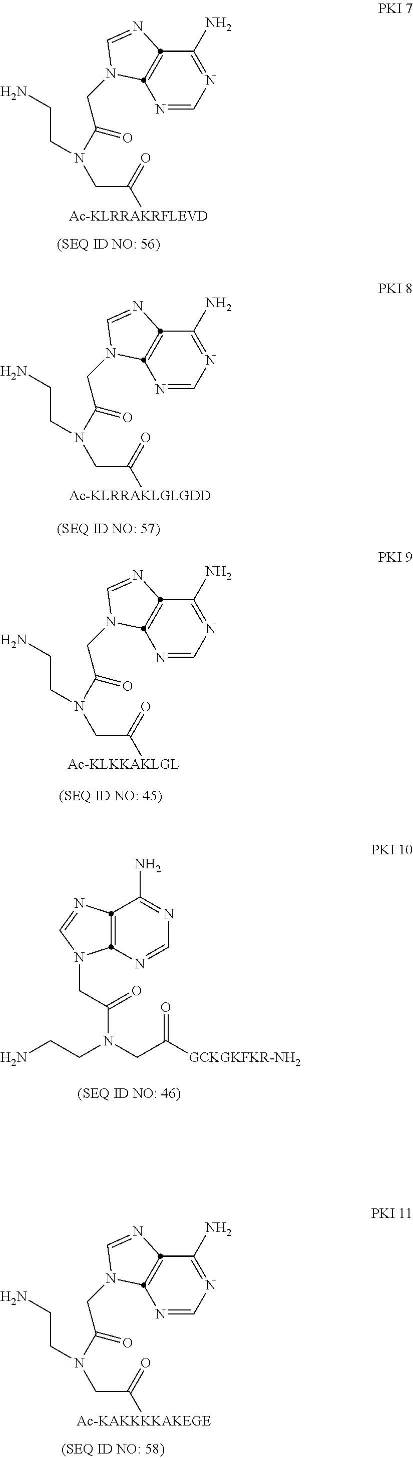

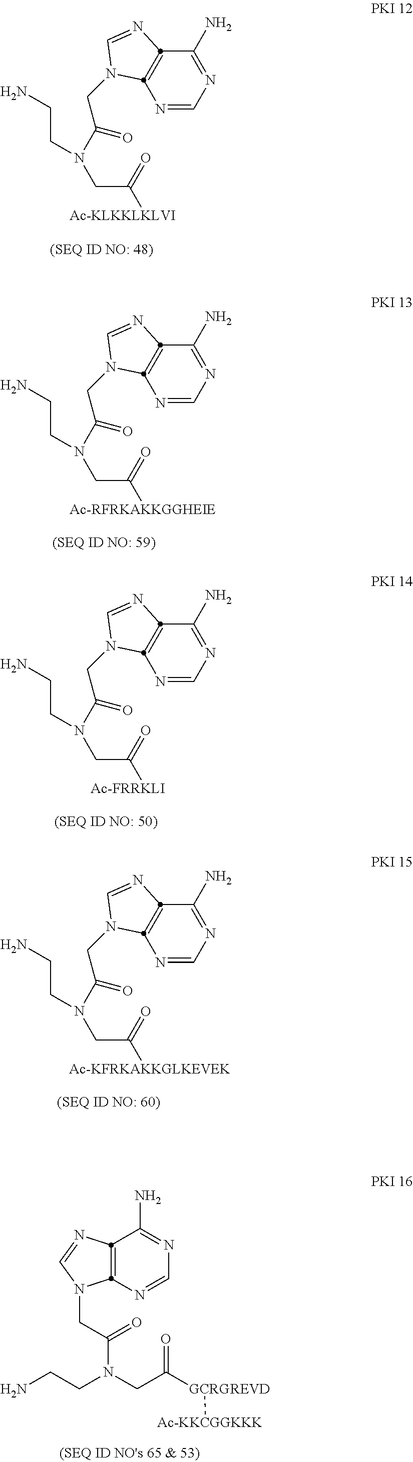

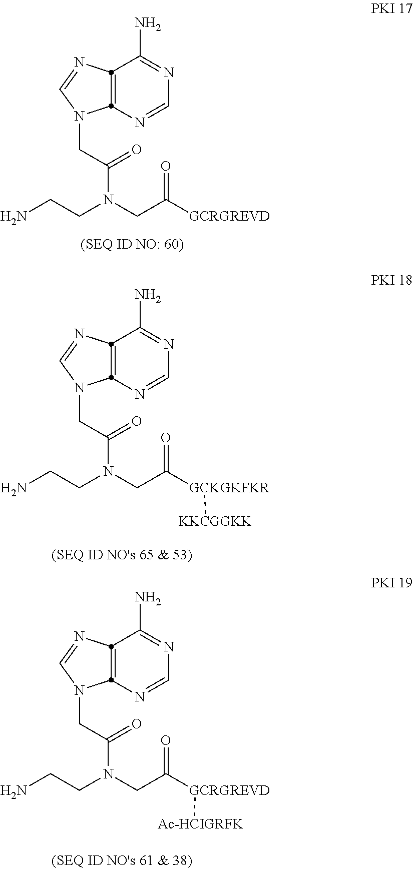

20. The targeted PKC inhibitor according to claim 15, wherein said targeted PKC inhibitor is selected from:

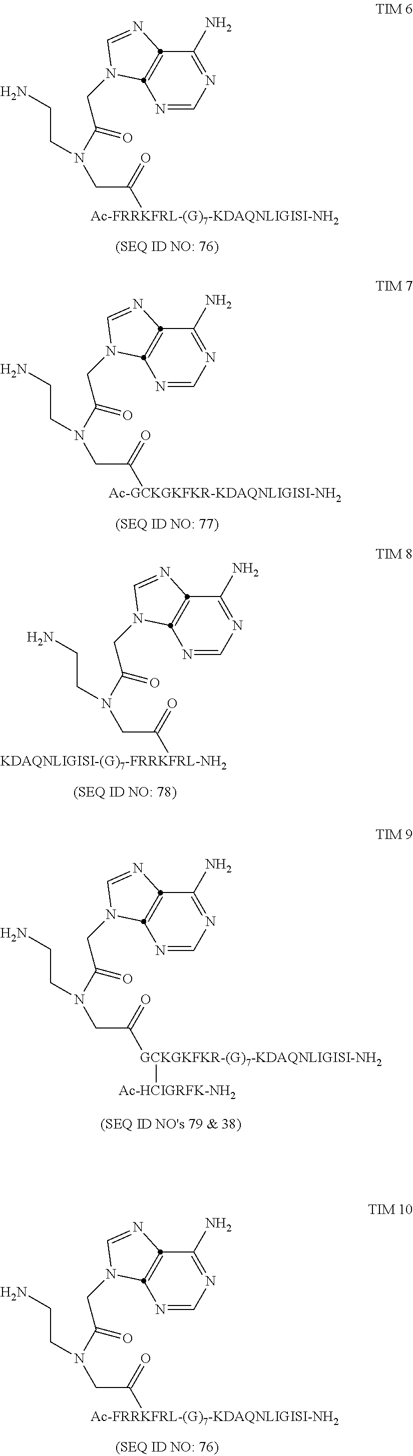

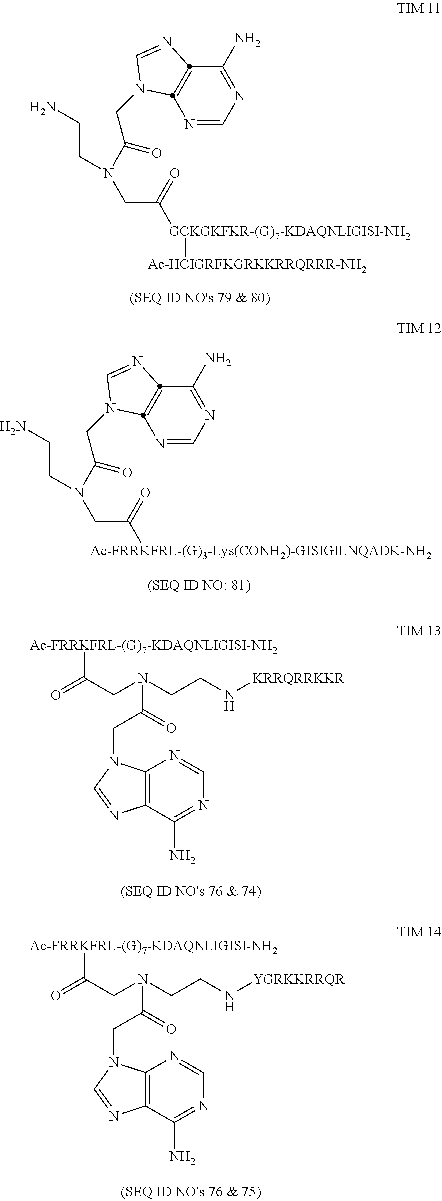

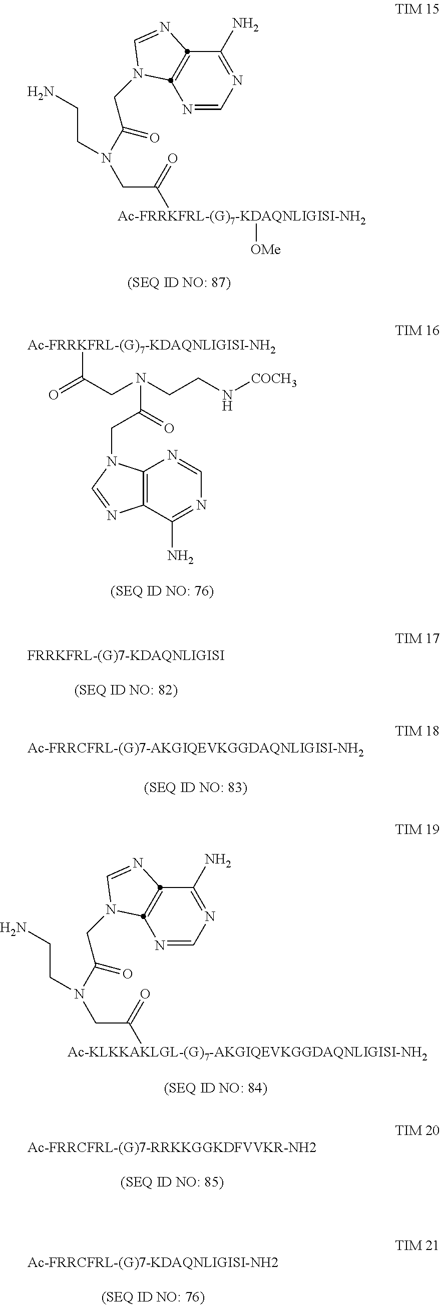

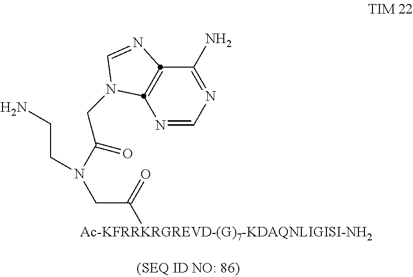

21. A pharmaceutical composition comprising the targeted protein kinase C inhibitor according to any one of claims 1, 2, 3, 4, 5, 6, 7, 8, 9, 10, 11, 12, 13, 14, 15, 16, 17, 18, 19 or 20, and a pharmaceutically acceptable diluent, carrier or excipient.

22. The targeted protein kinase C inhibitor according to any one of claims 1, 2, 3, 4, 5, 6, 7, 8, 9, 10, 11, 12, 13, 14, 15, 16, 17, 18, 19 or 20, for use in the treatment of a protein kinase C (PKC)-related disease or disorder.

23. The targeted protein kinase C inhibitor according to claim 22, wherein said PKC-related disease or disorder is cancer, a disorder associated with diabetes, or a cardiovascular disease or disorder.

24. The targeted protein kinase C inhibitor according to any one of claims 1, 2, 3, 4, 5, 6, 7, 8, 9, 10, 11, 12, 13, 14, 15, 16, 17, 18, 19 or 20, for use in the treatment of cancer.

25. The targeted protein kinase C inhibitor according to claim 24, wherein said cancer is colon cancer, colorectal cancer or breast cancer.

26. The targeted protein kinase C inhibitor according to claim 24 or 25, wherein said cancer is a drug-resistant cancer.

27. The targeted protein kinase C inhibitor according to any one of claims 24, 25 or 26, wherein said use is in combination with a chemotherapeutic agent.

28. Use of a targeted protein kinase C inhibitor according to any one of claims 1, 2, 3, 4, 5, 6, 7, 8, 9, 10, 11, 12, 13, 14, 15, 16, 17, 18, 19 or 20, for the manufacture of a medicament.

29. The use according to claim 28, wherein said medicament is for the treatment of a protein kinase C (PKC)-related disease or disorder.

30. The use according to claim 29, wherein said PKC-related disease or disorder is cancer, a disorder associated with diabetes, or a cardiovascular disease or disorder.

31. The vise according to claim 28, wherein said medicament is for the treatment of cancer.

32. The use according to claim 31, wherein said cancer is colon cancer, colorectal cancer or breast cancer.

33. The use according to claim 31 or 32, wherein said cancer is a drug-resistant cancer.

34. The use according to any one of claims 31, 32 or 33, wherein said treatment is in combination with a chemotherapeutic.

35. A method of inhibiting one or more protein kinase C isoforms, said method comprising contacting said one or more PKC isoforms with an effective amount of the targeted PKC inhibitor according to any one of claims 1, 2, 3, 4, 5, 6, 7, 8, 9, 10, 11, 12, 13, 14, 15, 16, 17, 18, 19 or 20.

36. The method according to claim 35, wherein said one or more PKC isoforms are selected from the group of: PKC-alpha, PKC-beta I, PKC-beta II, PKC-delta, PKC-epsilon, PKC-iota and PKC-zeta.

37. The method according to claim 35, wherein said one or more PKC isoforms are selected from the group of: PKC-alpha, PKC-beta I, PKC-beta II, PKC-delta and PKC-epsilon.

38. The method according to claim 35, wherein said PKC isoform is PKC-alpha.

39. The method according to any one of claims 35, 36, 37 or 38, wherein said method is an in vitro method.

40. The method according to any one of claims 35, 36, 37 or 38, wherein said method is an in vivo method.

41. A method of treating a mammal having a protein kinase C-related disease or disorder comprising administering to said mammal an effective amount of the targeted PKC inhibitor according to any one of claims 1, 2, 3, 4, 5, 6, 7, 8, 9, 10, 11, 12, 13, 14, 15, 16, 17, 18, 19 or 20.

42. The method according to claim 41, wherein said PKC-related disease or disorder is cancer, a disorder associated with diabetes, or a cardiovascular disease or disorder.

43. A method of treating a mammal having cancer comprising administering to said mammal an effective amount of the targeted PKC inhibitor according to any one of claims 1, 2, 3, 4, 5, 6, 7, 8, 9, 10, 11, 12, 13, 14, 15, 16, 17, 18, 19 or 20.

44. The method according to claim 43, wherein said cancer is colon cancer, colorectal cancer or breast cancer.

45. The method according to claim 43 or 44, wherein said cancer is a drug-resistant cancer.

46. The method according to any one of claims 43, 44 or 45, wherein said targeted PKC inhibitor is administered in combination with a chemotherapeutic agent.

47. A method of increasing the efficacy of a chemotherapeutic agent in a mammal having cancer and undergoing treatment with said chemotherapeutic agent, said method comprising administering to said mammal an effective amount of the targeted PKC inhibitor according to any one of claims 1, 2, 3, 4, 5, 6, 7, 8, 9, 10, 11, 12, 13, 14, 15, 16, 17, 18, 19 or 20.

48. The method according to claim 47, wherein said cancer is colon cancer, colorectal cancer or breast cancer.

49. The method according to claim 47 or 48, wherein said cancer is a drug-resistant cancer.


SEQUENCE LISTING (87 sequences)

SEQ ID NO: 1 - protein, 13 aa, Artificial Sequence
  Synthetic construct Peptide PRE #1
RRKKGGKDFVVKR

SEQ ID NO: 2 - protein, 11 aa, Artificial Sequence
  Synthetic construct Peptide PRE #14
KDAQNLIGISI

SEQ ID NO: 3 - protein, 11 aa, Artificial Sequence
  Synthetic construct Peptide PRE #15
KDANQLIGISI

SEQ ID NO: 4 - protein, 20 aa, Artificial Sequence
  Synthetic construct Peptide PRE # 16
AKGIQEVKGGDAQNLIGISI

SEQ ID NO: 5 - protein, 17 aa, Artificial Sequence
  Synthetic construct Peptide PRE # 8
ILEDKGGDAQNLIGISI

SEQ ID NO: 6 - protein, 11 aa, Artificial Sequence
  Synthetic construct Peptide PRE # 17
RDAQNLIGISI

SEQ ID NO: 7 - protein, 21 aa, Artificial Sequence
  Synthetic construct Peptide PRE # 18
AKGIQEVKGGKDAQNLIGISI

SEQ ID NO: 8 - protein, 11 aa, Artificial Sequence
  Synthetic construct Peptide PRE # 19
KDAQNLIGISL

SEQ ID NO: 9 - protein, 7 aa, Artificial Sequence
  Synthetic construct Peptide PRE # 20
KDAQNLI

SEQ ID NO: 10 - protein, 7 aa, Artificial Sequence
  Synthetic construct Peptide PRE # 21
RDAQNLI

SEQ ID NO: 11 - protein, 11 aa, Artificial Sequence
  Synthetic construct Peptide PRE # 2
KDAQNLIGISL

SEQ ID NO: 12 - protein, 20 aa, Artificial Sequence
  Synthetic construct Peptide PRE # 3
AKGIQEVKGGDAQNLIGISI

SEQ ID NO: 13 - protein, 11 aa, Artificial Sequence
  Synthetic construct Peptide PRE # 4
KDAQNLIGISI

SEQ ID NO: 14 - protein, 21 aa, Artificial Sequence
  Synthetic construct Peptide PRE # 5
AKGIQEVKGGKDAQNLIGISI

SEQ ID NO: 15 - protein, 12 aa, Artificial Sequence
  Synthetic construct Peptide PRE # 22
XKDAQNLIGISI

SEQ ID NO: 16 - protein, 11 aa, Artificial Sequence
  Synthetic construct Peptide PRE # 6
KDANQLIGISI

SEQ ID NO: 17 - protein, 11 aa, Artificial Sequence
  Synthetic construct Peptide PRE # 7
ISIGILQNADK

SEQ ID NO: 18 - protein, 11 aa, Artificial Sequence
  Synthetic construct Peptide PRE # 9 with all amino acids being D-amino acids
ISIGILQNADK

SEQ ID NO: 19 - protein, 11 aa, Artificial Sequence
  Synthetic construct Peptide PRE # 10
ISIGILNQADK

SEQ ID NO: 20 - protein, 11 aa, Artificial Sequence
  Synthetic construct Peptide PRE # 11
RDAQNLIGISI

SEQ ID NO: 21 - protein, 7 aa, Artificial Sequence
  Synthetic construct Peptide PRE # 12
KDAQNLI

SEQ ID NO: 22 - protein, 7 aa, Artificial Sequence
  Synthetic construct Peptide PRE # 13
RDAQNLI

SEQ ID NO: 23 - protein, 11 aa, Artificial Sequence
  Synthetic construct Peptide PRE # 23
ISIGILQNADK

SEQ ID NO: 24 - protein, 11 aa, Artificial Sequence
  Synthetic construct Peptide PRE # 24 with all amino acids being D-amino acids
ISIGILQNADK

SEQ ID NO: 25 - protein, 11 aa, Artificial Sequence
  Synthetic construct Peptide PRE # 25
ISIGILNQADK

SEQ ID NO: 26 - protein, 11 aa, Artificial Sequence
  Synthetic construct Peptide PRE # 26
RRRRGQQNNLS

SEQ ID NO: 27 - protein, 13 aa, Artificial Sequence
  Synthetic construct Peptide PRE # 27
KKKKGGNLVKRIL

SEQ ID NO: 28 - protein, 25 aa, Artificial Sequence
  Synthetic construct Peptide PRE # 28
ARIQQEILKKRGGGKDAQNLIGISL

SEQ ID NO: 29 - protein, 21 aa, Artificial Sequence
  Synthetic construct Peptide PRE # 29
ARGIQEFRGGKEAQNLVISIL

SEQ ID NO: 30 - protein, 11 aa, Artificial Sequence
  Synthetic construct Peptide PRE # 30
REAQNLIGISI

SEQ ID NO: 31 - protein, 10 aa, Artificial Sequence
  Synthetic construct Peptide PRE # 31
EAQNLIGISI

SEQ ID NO: 32 - protein, 11 aa, Artificial Sequence
  Synthetic construct Peptide PRE # 32
EAQNVIVISIL

SEQ ID NO: 33 - protein, 6 aa, Artificial Sequence
  Synthetic construct Peptide PRE # 33
EAQVSI

SEQ ID NO: 34 - protein, 7 aa, Artificial Sequence
  Synthetic construct Peptide PRE # 34
KAQNISI

SEQ ID NO: 35 - protein, 9 aa, Artificial Sequence
  Synthetic construct Peptide PRE # 35
RDAQVVRIV

SEQ ID NO: 36 - protein, 7 aa, Artificial Sequence
  Synthetic construct peptide
LRRAKLG

SEQ ID NO: 37 - protein, 7 aa, Artificial Sequence
  Synthetic construct peptide
FRRKFRL

SEQ ID NO: 38 - protein, 7 aa, Artificial Sequence
  Synthetic construct peptide
HCIGRFK

SEQ ID NO: 39 - protein, 8 aa, Artificial Sequence
  Synthetic construct peptide
GCKGKFKR

SEQ ID NO: 40 - protein, 8 aa, Artificial Sequence
  Synthetic construct peptide
KFRRKRGR

SEQ ID NO: 41 - protein, 8 aa, Artificial Sequence
  Synthetic construct peptide
KFRRKLRL

SEQ ID NO: 42 - protein, 9 aa, Artificial Sequence
  Synthetic construct peptide
KLRRAKRFL

SEQ ID NO: 43 - protein, 7 aa, Artificial Sequence
  Synthetic construct peptide
FRRCFRL

SEQ ID NO: 44 - protein, 10 aa, Artificial Sequence
  Synthetic construct peptide
KLRRAKLGLG

SEQ ID NO: 45 - protein, 9 aa, Artificial Sequence
  Synthetic construct peptide
KLKKAKLGL

SEQ ID NO: 46 - protein, 8 aa, Artificial Sequence
  Synthetic construct peptide
GCKGKFKR

SEQ ID NO: 47 - protein, 8 aa, Artificial Sequence
  Synthetic construct peptide
KAKKKKAK

SEQ ID NO: 48 - protein, 9 aa, Artificial Sequence
  Synthetic construct peptide
KLKKLKLVI

SEQ ID NO: 49 - protein, 10 aa, Artificial Sequence
  Synthetic construct peptide
RFRKAKKGGH

SEQ ID NO: 50 - protein, 6 aa, Artificial Sequence
  Synthetic construct peptide
FRRKLI

SEQ ID NO: 51 - protein, 10 aa, Artificial Sequence
  Synthetic construct peptide
KFRKAKKGLK

SEQ ID NO: 52 - protein, 5 aa, Artificial Sequence
  Synthetic construct peptide
GCRGR

SEQ ID NO: 53 - protein, 8 aa, Artificial Sequence
  Synthetic construct peptide
KKCGGKKK

SEQ ID NO: 54 - protein, 11 aa, Artificial Sequence
  Synthetic construct peptide
KFRRKRGREVD

SEQ ID NO: 55 - protein, 11 aa, Artificial Sequence
  Synthetic construct peptide
KFRRKLRLEVD

SEQ ID NO: 56 - protein, 12 aa, Artificial Sequence
  Synthetic construct peptide
KLRRAKRFLEVD

SEQ ID NO: 57 - protein, 12 aa, Artificial Sequence
  Synthetic construct peptide
KLRRAKLGLGDD

SEQ ID NO: 58 - protein, 11 aa, Artificial Sequence
  Synthetic construct peptide
KAKKKKAKEGE

SEQ ID NO: 59 - protein, 13 aa, Artificial Sequence
  Synthetic construct peptide
RFRKAKKGGHEIE

SEQ ID NO: 60 - protein, 14 aa, Artificial Sequence
  Synthetic construct peptide
KFRKAKKGLKEVEK

SEQ ID NO: 61 - protein, 8 aa, Artificial Sequence
  Synthetic construct peptide
GCRGREVD

SEQ ID NO: 62 - DNA, 81 bp, Artificial Sequence
  Synthetic construct Coding oligonucleotide for TIM 17
atgtttcgccgcaaatttcgcctgggcggcggcggcggcggcggcaaagatgcgcagaacctgattggcattagcatttga

SEQ ID NO: 63 - DNA, 81 bp, Artificial Sequence
  Synthetic construct Complementary oligonucleotide for TIM 17
tcaaatgctaatgccaatcaggttctgcgcatctttgccgccgccgccgccgccgcccaggcgaaatttgcggcgaaacat

SEQ ID NO: 64 - DNA, 49 bp, Artificial Sequence
  Sense primer
ggggacaagtttgtacaaaaaagcaggcttgatgtttcgccgcaaattt

SEQ ID NO: 65 - DNA, 47 bp, Artificial Sequence
  Antisense primer
ggggaccactttgtacaagaaagctgggtctaaatgctaatgccaat

SEQ ID NO: 66 - DNA, 139 bp, Artificial Sequence
  Synthetic construct Sequence encoding TIM 17
ggggacaagtttgtacaaaaaagcaggcttgatgtttcgccgcaaattcgcctgggcggcggcggcggcggcggcaaagatgcgcagaacctgattgcattagcatttagacccagctttcttgtacaaagtggtcccc

SEQ ID NO: 67 - protein, 52 aa, Artificial Sequence
  Synthetic construct
XXXXXXXXXXXXXXXXXXXXXXXXGGGGXXXXXXXXXXXXXXXXXXXXXXXX

SEQ ID NO: 68 - protein, 5 aa, Artificial Sequence
  Synthetic construct
ATPKL

SEQ ID NO: 69 - protein, 5 aa, Artificial Sequence
  Synthetic construct
LKPTA

SEQ ID NO: 70 - protein, 52 aa, Artificial Sequence; identical to SEQ ID NO: 67
  Synthetic construct

SEQ ID NO: 71 - protein, 4 aa, Artificial Sequence
  Synthetic construct
EVEK

SEQ ID NO: 72 - protein, 16 aa, Drosophila antennapedia
RQIKIWFQNRRMKWKK

SEQ ID NO: 73 - protein, 9 aa, Human immunodeficiency virus
RRRQRRKKR

SEQ ID NO: 74 - protein, 9 aa, Human immunodeficiency virus
KRRQRRKKR

SEQ ID NO: 75 - protein, 9 aa, Artificial Sequence
  Synthetic construct
YGRKKRRQR

SEQ ID NO: 76 - protein, 25 aa, Artificial Sequence
  Synthetic construct
FRRKFRLGGGGGGGKDAQNLIGISI

SEQ ID NO: 77 - protein, 19 aa, Artificial Sequence
  Synthetic construct
GCKGKFKRKDAQNLIGISI

SEQ ID NO: 78 - protein, 25 aa, Artificial Sequence
  Synthetic construct
KDAQNLIGISIGGGGGGGFRRKFRL

SEQ ID NO: 79 - protein, 26 aa, Artificial Sequence
  Synthetic construct
GCKGKFKRGGGGGGGKDAQNLIGISI

SEQ ID NO: 80 - protein, 17 aa, Artificial Sequence
  Synthetic construct
HCIGRFKGRKKRRQRRR

SEQ ID NO: 81 - protein, 23 aa, Artificial Sequence
  Synthetic construct
FRRKFRLGGGKGISIGILNQADK

SEQ ID NO: 82 - protein, 25 aa, Artificial Sequence
  Synthetic construct
FRRKFRLGGGGGGGKDAQNLIGISI

SEQ ID NO: 83 - protein, 34 aa, Artificial Sequence
  Synthetic construct
FRRCFRLGGGGGGGAKGIQEVKGGDAQNLIGISI

SEQ ID NO: 84 - protein, 36 aa, Artificial Sequence
  Synthetic construct
KLKKAKLGLGGGGGGGAKGIQEVKGGDAQNLIGISI

SEQ ID NO: 85 - protein, 27 aa, Artificial Sequence
  Synthetic construct
FRRCFRLGGGGGGGRRKKGGKDFVVKR

SEQ ID NO: 86 - protein, 29 aa, Artificial Sequence
  Synthetic construct
KFRRKRGREVDGGGGGGGKDAQNLIGISI

SEQ ID NO: 87 - protein, 25 aa, Artificial Sequence
  Synthetic construct
FRRKFRLGGGGGGGKDAQNLIGISI